(19) United States
(12) Patent Application Publication
(10) Pub. No.: US 2022/0127635 A1
(43) Pub. Date: Apr. 28, 2022

(54) METHOD OF INCREASING RESISTANCE AGAINST SOYBEAN RUST IN TRANSGENIC PLANTS BY EXPRESSION OF A SUGAR TRANSPORTER

(71) Applicant: BASF SE, Ludwigshafen (DE)

(72) Inventors: Brody John DeYoung, Research Triangle Park, NC (US); Holger Schultheiss, Limburgerhof (DE); Ralf Flachmann, Limburgerhof (DE); David A. Hubert, Research Triangle Park, NC (US); Petra Epple, Research Triangle Park, NC (US)

(21) Appl. No.: 17/437,929

(22) PCT Filed: Mar. 2, 2020
(86) PCT No.: PCT/EP2020/055423

§ 371 Date: Sep. 10, 2021

Related U.S. Application Data
(60) Provisional application No. 62/817,258, filed on Mar. 12, 2019.

(51) Int. Cl. C12N 15/82

(52) U.S. Cl. CPC C12N 15/8282; C12N 15/8213

(57) ABSTRACT

The present invention relates to an Lr67 allele differing from a soybean Lr67 wild type gene as defined herein. The allele unexpectedly conveys, intensifies or stabilizes fungal pathogen tolerance or resistance to a plant, seed, cell, or other plant part, most preferably against rusts of genus Phakopsora in leguminous plants or parts thereof. The invention in particular provides cells, expression constructs and plants and parts thereof, for example seed, comprising the Lr67 allele, and products obtainable or obtained therefrom. The invention also provides ensembles or populations of plants or accumulations of seed. Furthermore, the invention provides uses and methods to make use of the Lr67 allele of the invention and to realize the advantages conferred thereby. In particular, the invention provides methods for creating a cell, plant or part thereof comprising the Lr67 allele, and methods for producing a plant population having resistance against a fungal pathogen. Further methods according to the invention relate to the reduction or abolition of susceptibility of a plant or part thereof to infections by a fungal pathogen, for assaying the susceptibility to fungal pathogen infection and for preparation of feed or food products. The Lr67 allele of the invention can be incorporated into the plant transgenically or via man-directed mutagenesis.

### FIELD OF THE INVENTION

The present invention relates to an Lr67 allele differing from a soybean Lr67 wild type gene as defined herein. The allele unexpectedly conveys, intensifies or stabilizes fungal pathogen tolerance or resistance to a plant, seed, cell, or other plant part, most preferably against rusts of genus Phakopsora in leguminous plants or parts thereof. The invention in particular provides cells, expression constructs and plants and parts thereof, for example seed, comprising the Lr67 allele, and products obtainable or obtained therefrom. The invention also provides ensembles or populations of plants or accumulations of seed. Furthermore, the invention provides uses and methods to make use of the Lr67 allele of the invention and to realize the advantages conferred thereby. In particular, the invention provides methods for creating a cell, plant or part thereof comprising the Lr67 allele, and methods for producing a plant population having resistance against a fungal pathogen. Further methods according to the invention relate to the reduction or abolition of susceptibility of a plant or part thereof to infections by a fungal pathogen, for assaying the susceptibility to fungal pathogen infection and for preparation of feed or food products. The Lr67 allele of the invention can be incorporated into the plant transgenically or via man-directed mutagenesis.

### BACKGROUND OF THE INVENTION

Plant pathogenic organisms and particularly fungi have resulted in severe reductions in crop yield in the past, in worst cases leading to famine. Monocultures in particular are highly susceptible to an epidemic-like spreading of diseases. To date, the pathogenic organisms have been controlled mainly by using pesticides. Currently the possibility of directly modifying the genetic disposition of a plant or pathogen is also open to man. Alternatively, naturally occurring fungicides produced by the plants after fungal infection can be synthesized and applied to the plants.

The term “resistance” as used herein refers to an absence or reduction of one or more disease symptoms in a plant caused by a plant pathogen. Resistance generally describes the ability of a plant to prevent, or at least curtail the infestation and colonization by a harmful pathogen. Different mechanisms can be discerned in the naturally occurring resistance, with which the plants fend off colonization by phytopathogenic organisms (Schopfer and Brennicke (1999) Pflanzenphysiologie, Springer Verlag, Berlin-Heidelberg, Germany).

With regard to the race specific resistance, also called host resistance, a differentiation is made between compatible and incompatible interactions. In the compatible interaction, an interaction occurs between a virulent pathogen and a susceptible plant. The pathogen survives and may build up reproductive structures, while the host is seriously hampered in development or dies off. An incompatible interaction occurs, on the other hand, when the pathogen infects the plant but is inhibited in its growth before or after weak development of symptoms (mostly by the presence of Resistance (R) genes of the NBS-LRR family, see below). In the latter case, the plant is resistant to the respective pathogen (Schopfer and Brennicke, vide supra). However, this type of resistance is mostly specific for a certain strain or pathogen.

In both compatible and incompatible interactions, a defensive and specific reaction of the host to the pathogen occurs. In nature, however, this resistance is often overcome because of the rapid evolutionary development of new virulent races of the pathogens (Neu et al. (2003) American Cytopathol. Society, MPMI 16 No. 7: 626-633).

Most pathogens are plant-species specific. This means that a pathogen can induce a disease in a certain plant species, but not in other plant species (Heath (2002) Can. J. Plant Pathol. 24: 259-264). The resistance against a pathogen in certain plant species is called non-host resistance. The non-host resistance offers strong, broad, and permanent protection from phytopathogens. Genes providing non-host resistance provide the opportunity of a strong, broad and permanent protection against certain diseases in non-host plants. In particular, such a resistance works for different strains of the pathogen.

Fungi are distributed worldwide. Approximately 100 000 different fungal species are known to date. Thereof, rusts are of great importance. They can have a complicated development cycle with up to five different spore stages (spermatium, aecidiospore, uredospore, teleutospore and basidiospore).

During the infection of plants by pathogenic fungi, different phases are usually observed. The first phases of the interaction between phytopathogenic fungi and their potential host plants are decisive for the colonization of the plant by the fungus. During the first stage of the infection, the spores become attached to the surface of the plants, germinate, and the fungus penetrates the plant. Fungi may penetrate the plant via existing ports such as stomata, lenticels, hydathodes and wounds, or else they penetrate the plant epidermis directly as the result of mechanical force with the aid of cell-wall-digesting enzymes. Specific infection structures are developed for penetration of the plant. To counteract, plants have developed physical barriers, such as wax layers, and chemical compounds having antifungal effects to inhibit spore germination, hyphal growth or penetration.

The soybean rust Phakopsora pachyrhizi directly penetrates the plant epidermis. After growing through the epidermal cell, the fungus reaches the intercellular space of the mesophyll, where the fungus starts to spread through the leaf. To acquire nutrients, the fungus penetrates mesophyll cells and develops haustoria inside the mesophyll cells. During the penetration process the plasma membrane of the penetrated mesophyll cell stays intact. It is a particularly troubling feature of Phakopsora rusts that these pathogens exhibit an immense variability, thereby overcoming novel plant resistance mechanisms and novel fungicide activities within a few years and sometimes already within one Brazilian growing season.

Fusarium species are important plant pathogens that attacks a wide range of plant species including many important crops such as maize and wheat. They cause seed rots and seedling blights as well as root rots, stalk rots and ear rots. Pathogens of the genus Fusarium infect the plants via roots, silks or previously infected seeds or they penetrate the plant via wounds or natural openings and cracks. After a very short establishment phase the Fusarium fungi start to secrete mycotoxins such as trichothecenes, zearalenone and fusaric acid into the infected host tissues leading to cell death and maceration of the infected tissue. Feeding on dead tissue, the fungus then starts to spread through the infected plant leading to severe yield losses and decreases in quality of the harvested grain.

Biotrophic phytopathogenic fungi depend for their nutrition on the metabolism of living plant cells. This type of fungi belongs to the group of biotrophic fungi, like many rust fungi, powdery mildew fungi or oomycete pathogens like the genus Phytophthora or Peronospora. Necrotrophic phytopathogenic fungi depend for their nutrition on dead cells of the plants, e.g. species from the genus Fusarium, Rhizoctonia or Mycospaerella. Soybean rust occupies an intermediate position. It penetrates the epidermis directly, whereupon the penetrated cell becomes necrotic. However, after penetration, the fungus changes over to an obligate-biotrophic lifestyle. The subgroup of the biotrophic fungal pathogens which follows essentially such an infection strategy are heminecrotrophic.

Recently in wheat, a specific allele of the leaf rust resistance gene Lr67 (LR67res) has been described which conveys adult plant resistance to leaf rust and stripe rust (Hiebert et al., Theor Appl Genet. 2010 October; 121(6):1083-91). Transgenic expression of the wheat Lr67res genes in other cereal species confers resistance to multiple pathogens of these crops (Spielmeyer et al., BMC Plant Biology 2013, 13:96; Milne et al., Plant Physiology 2018; Periyannan, PLoS Pathog 13(7): e1006380). However, expression of the wheat Lr67res gene in soy did not result in significant reduction of susceptibility of adult soy plants to infections by rust fungi, and in particular did not lead to a statistically significant resistance against Phakopsora pachyrhizi.

The present invention thus set out to provide materials and methods or uses of material suitable in the context of conferring, intensifying or stabilizing resistance against fungal pathogen infections. The materials and methods should lead to plant material of heritably improved resistance against infection by a fungal pathogen, preferably a rust fungus and most preferably a rust fungus of genus Phakopsora. The invention also should provide correspondingly improved plants and plant material and methods for raising populations of plants or creating accumulations of seed, most preferably for creating further products therefrom.

### SUMMARY OF THE INVENTION

The invention thus provides a cell having an Lr67 allele comprising one or more mutations in a soybean Lr67 gene, wherein the one or more mutations comprise, in the numbering according to SEQ ID NO. 1, a substitution at position G145, preferably a substitution selected from, in decreasing order of preference, G145R, G145K, G145H, G145Q, G145E, G145V, G145L and G145Y, and optionally also a substitution at position 1389, preferably a substitution selected from, in decreasing order of preference, 1389L, 1389W, 1389K, 1389R, 1389Q, 1389F and 1389M.

The invention also provides an expression construct comprising an Lr67 allele comprising one or more mutations in a soybean Lr67 gene as defined herein, operably linked to a heterologous polynucleotide.

And the invention provides a plant or plant part comprising a cell according to the invention or transformed with an expression construct according to the invention.

Furthermore, an ensemble of at least 50 plants according to invention is provided, and also a seed, flower, leaf, fruit, processed food, or food ingredient from the plant according to the invention. Likewise, the invention provides an accumulation of at least 1 kg of seeds from one or more plants according to the invention.

A plant or an ensemble according to the invention, or a part of such plant or ensemble, can beneficially be used as animal feed or to produce a feed product for animal or a food product for human consumption.

The invention also provides a method for creating a cell according to the invention, comprising the step of transforming a cell with a nucleic acid coding for an Lr67 allele as described according to the invention.

The invention furthermore provides a method for creating a cell according to the invention, comprising the steps of

i) introducing into a cell an enzyme having endonuclease or nickase activity and recognizing a soybean Lr67 gene sequence,

ii) effecting by action of said enzyme a change in nucleotide sequence such as to introduce an amino acid change at position 145 and optionally also at position 389, using the numbering according to SEQ ID NO. 1.

For producing a population of plants each having an enhanced resistance to at least one biotrophic or heminecrotrophic fungus the invention provides a method comprising the steps of

i) providing a plant according to the invention, and

ii) crossing the plant of step a) with a compatible plant without active Lr67 allele, and

iii) growing progeny obtained by the crossing of step b).

The invention also provides a method for reducing or abolishing susceptibility of a plant or plant part, in comparison to a wild type plant, to infections by a biotrophic or heminecrotrophic fungus, preferably a rust fungus, comprising causing or increasing expression or activity of an Lr67 allele comprising one or more mutations in a soybean Lr67 gene, wherein the one or more mutations comprise, in the numbering according to SEQ ID NO. 1, a substitution at position G145, preferably a substitution selected from, in decreasing order of preference, G145R, G145K, G145H, G145Q, G145E, G145V, G145L and G145Y, and optionally also a substitution at position 1389, preferably a substitution selected from, in decreasing order of preference, 1389L, 1389W, 1389K, 1389R, 1389Q, 1389F and 1389M.

Furthermore, the invention provides a method for plant protection against one or more fungal pathogens, comprising the steps of

a) planting a plant according to the present invention, and

b) applying, to at least one of (a) the plant, (b) an area adjacent to the plant, (c) soil adapted to support growth of the plant, (d) a root of the plant, and (e) foliage of the plant, a fungicidally effective amount of a composition comprising one or more fungicides effective against one or more fungi, preferably effective against necrotrophic fungi.

And the invention provides a method of assaying a plant for resistance to a biotrophic or heminecrotrophic fungus, preferably a rust fungus, comprising the screening for the presence of the Lr67 allele in a cell of said plant, preferably comprising the detection of a polynucleotide, preferably an mRNA,

a) hybridizing under stringent conditions to a nucleic acid probe according to SEQ ID NO. 27, 28, 29, 30, 31 or 32, and/or

b) having at least 60% identity to any of SEQ ID NO. 33, 34, 35, 36, 37 or 38.

Also, the invention provides a method of propagation of a sexually reproducing plant, comprising:
i) obtaining a plurality of seeds of a plant, preferably a monocotyledon or dicotyledon plant, more preferably a plant of order Fabales, more preferably a plant of family Fabaceae, more preferably a plant of tribus Phaseoleae, more preferably of genus Glycine, most preferably of species Glycine max, Glycine soja, Glycine gracilis or a cross Glycine max×Glycine soja,
ii) ascertaining that at least about 25%, more preferably at least 50%, even more preferably 25%-95%, even more preferably 25%-100% and most preferably 45%-100% of the seed of one species comprise an Lr67 allele according to the present invention, and
iii) planting seed of the plurality of seeds satisfying the condition according to step ii).

The invention is hereinafter described in more detail. The accompanying figures and examples provided herein are intended not to limit the scope of the invention or of the claims.

### BRIEF DESCRIPTION OF FIGURES

FIG. 1 shows the scoring system used to determine the level of diseased leaf area of wildtype and transgenic soy plants against the rust fungus P. pachyrhizi (as described in GODOY, C. V., KOGA, L. J. & CANTERI, M. G. Diagrammatic scale for assessment of soybean rust severity. Fitopatologia Brasileira 31:063-068. 2006).

FIG. 2 shows an alignment of the artificial soybean Lr67 gene sequence SEQ ID NO.1, the soybean LR67 gene sequences SEQ ID NO. 2-7, the Lr67res allele of WO2015024066 (“TaLr67res”) and the Lr67 alleles SEQ ID NO. 8-14 according to the present invention. The amino acid sequence is given only for the top sequence SEQ ID NO. 1, for every other sequence only the differing amino acids or “-” for a gap are indicated (“.” denotes “same amino acid as in top sequence”).

FIG. 3 shows a plot of positional amino acid conservation. Per columns: The number of stars indicates the degree of conservation (more stars, higher degree of conservation); the first letter/“−” below the stars is the respective amino acid encountered in SEQ ID NO. 1; all letters/“−” below indicate, in decreasing order of frequency, the amino acids encountered in homologous Lr67 genes.

FIG. 4 shows the nucleic acid of the TaLr67res allele of WO2015024066.

FIG. 5a shows a schematic illustration of the plant transformation vector designed to overexpress TaLr67res under control of the parsley ubiquitine promoter.

FIG. 5b shows a schematic illustration of the plant transformation vector designed to overexpress Glyma.01g238800.1G145R_V389L under control of the parsley ubiquitine promoter as used in this invention as described in example 2. The skilled person understands that the name “Glyma.01g238800.1G145R_V387L” is a typographical error; the substitution is not at position 387 but at position 389 as described in the example and throughout this description and claims.

FIG. 6 shows the result of the disease scoring of transgenic soy plants expressing TaLr67res or Glyma.01g238800.1G145R_V389L (SEQ ID NO. 9) in T1 generation in greenhouse. At all 46 transgenic T1 soybean plants (from 4 independent events) expressing Glyma.01g238800.1G145R_V389L and 36 transgenic T1 soybean plants (from 3 independent events) expressing TaLr67res were inoculated with spores of Phakopsora pachyrhizi. The expression of Glyma.01g238800.1G145R_V389L and TaLr67res was checked by RT-PCR. The evaluation of the diseased leaf area on all leaves was performed 14 days after inoculation by imaging. The average of the percentage of the leaf area showing fungal colonies or strong yellowing/browning on all leaves was considered as diseased leaf area. Transgenic soybean plants expressing Glyma.01g238800.1G145R_V389L or TaLr67res respectively (expression checked by RT-PCR) were evaluated in parallel to 46 non-transgenic control plants. The relative diseased leaf area in comparison to control (set to 100%) is shown in FIG. 1. Overexpression of Glyma.01g238800.1G145R_V389L significantly (***: p<0.001) reduces the diseased leaf area in comparison to non-transgenic control plants by 70,8%. In contrast overexpression of TaLr67res did not lead to a statistically significant reduction of disease.

BRIEF DESCRIPTION OF SEQUENCES

| SEQ ID NO. | description |
|---|---|
| 1 | artificial sequence for sequence alignments |
| 2 | soybean wild type Lr67 gene |
| 3 | soybean wild type Lr67 gene |
| 4 | soybean wild type Lr67 gene |
| 5 | soybean wild type Lr67 gene |
| 6 | soybean wild type Lr67 gene |
| 7 | soybean wild type Lr67 gene |
| 8 | Lr67 allele based on SEQ ID NO. 1 |
| 9 | Lr67 allele based on SEQ ID NO. 2 |
| 10 | Lr67 allele based on SEQ ID NO. 3 |
| 11 | Lr67 allele based on SEQ ID NO. 4 |
| 12 | Lr67 allele based on SEQ ID NO. 5 |
| 13 | Lr67 allele based on SEQ ID NO. 6 |
| 14 | Lr67 allele based on SEQ ID NO. 7 |
| 15 | sense primer for SEQ ID NO. 2 |
| 16 | sense primer for SEQ ID NO. 3 |
| 17 | sense primer for SEQ ID NO. 4 |
| 18 | sense primer for SEQ ID NO. 5 |
| 19 | sense primer for SEQ ID NO. 6 |
| 20 | sense primer for SEQ ID NO. 7 |
| 21 | antisense primer for SEQ ID NO. 2 |
| 22 | antisense primer for SEQ ID NO. 3 |
| 23 | antisense primer for SEQ ID NO. 4 |
| 24 | antisense primer for SEQ ID NO. 5 |
| 25 | antisense primer for SEQ ID NO. 6 |
| 26 | antisense primer for SEQ ID NO. 7 |
| 27 | probe for SEQ ID NO. 2 |
| 28 | probe for SEQ ID NO. 3 |
| 29 | probe for SEQ ID NO. 4 |
| 30 | probe for SEQ ID NO. 5 |
| 31 | probe for SEQ ID NO. 6 |
| 32 | probe for SEQ ID NO. 7 |
| 33 | nucleic acid for wild type Lr67 gene SEQ ID NO. 2 |
| 34 | nucleic acid for wild type Lr67 gene SEQ ID NO. 3 |
| 35 | nucleic acid for wild type Lr67 gene SEQ ID NO. 4 |
| 36 | nucleic acid for wild type Lr67 gene SEQ ID NO. 5 |
| 37 | nucleic acid for wild type Lr67 gene SEQ ID NO. 6 |
| 38 | nucleic acid for wild type Lr67 gene SEQ ID NO. 7 |

### DETAILED DESCRIPTION OF THE INVENTION

The current invention is based on the identification of Lr67 alleles of a soybean Lr67 gene. Unexpectedly, these alleles convey or improve resistance against fungal pathogens in plants, in particular in dicotyledon plants, and in particular in soybean, whereas a known Lr67 allele of wheat did not alter fungal pathogen resistance to a statistically significant extent.

The technical teaching of the invention is expressed herein using the means of language, in particular by use of scientific and technical terms. However, the skilled person understands that the means of language, detailed and precise as they may be, can only approximate the full content of the technical teaching, if only because there are multiple ways of expressing a teaching, each necessarily failing to completely express all conceptual connections, as each expression necessarily must come to an end. With this in mind the skilled person understands that the subject matter of the invention is the sum of the individual technical concepts signified herein or expressed in a pars-pro-toto way by the innate constrains of a written description. In particular, the skilled person will understand that the signification of individual technical concepts is done herein as an abbreviation of spelling out each possible combination of concepts as far as technically sensible, such that for example the disclosure of three concepts or embodiments A, B and C are a shorthand notation of the concepts A+B, A+C, B+C, A+B+C. In particular, fallback positions for features are described herein in terms of lists of converging alternatives or instantiations. Unless stated otherwise, the invention described herein comprises any combination of such alternatives. The choice of more or less preferred elements from such lists is part of the invention and is due to the skilled person's preference for a minimum degree of realization of the advantage or advantages conveyed by the respective features. Such multiple combined instantiations represent the adequately preferred form(s) of the invention. Unless stated otherwise, preferred alternatives of features are listed herein in increasing order of preference.

As used herein, terms in the singular and the singular forms like “a”, “an” and “the” include plural referents unless the content clearly dictates otherwise. Thus, for example, reference to “plant”, “the plant” or “a plant” also includes a plurality of plants; also, depending on the context, use of the term “plant” can also include genetically similar or identical progeny of that plant or plants derived therefrom by crossing; use of the term “a nucleic acid” optionally includes, as a practical matter, many copies of that nucleic acid molecule; similarly, the term “probe” optionally (and typically) encompasses many similar or identical probe molecules. Also as used herein, the word “comprising” or variations such as “comprises” or “comprising” will be understood to imply the inclusion of a stated element, integer or step, or group of elements, integers or steps, but not the exclusion of any other element, integer or step, or group of elements, integers or steps.

As used herein, the term “and/or” refers to and encompasses any and all possible combinations of one or more of the associated listed items, as well as the lack of combinations when interpreted in the alternative (“or”). The term “comprising” also encompasses the term “consisting of”.

The term “about”, when used in reference to a measurable value, for example an amount of mass, dose, time, temperature, sequence identity and the like, refers to a variation of ±0.1%, 0.25%, 0.5%, 0.75%, 1%, 2%, 3%, 4%, 5%, 6%, 7%, 8%, 9%, 10%, 15% or even 20% of the specified value as well as the specified value. Thus, if a given composition is described as comprising “about 50% X,” it is to be understood that, in some embodiments, the composition comprises 50% X whilst in other embodiments it may comprise anywhere from 40% to 60% X (i.e., 50%±10%).

As used herein, the term “gene” refers to a biochemical information which, when materialised in a nucleic acid, can be transcribed into a gene product, i.e. a further nucleic acid, preferably an RNA, and preferably also can be translated into a peptide or polypeptide. The term is thus also used to indicate the section of a nucleic acid resembling said information and to the sequence of such nucleic acid (herein also termed “gene sequence”).

Also as used herein, the term “allele” refers to a variation of a gene characterized by one or more specific differences in the gene sequence compared to the wild type gene sequence, regardless of the presence of other sequence differences. Alleles or nucleotide sequence variants of the invention have at least, in increasing order of preference, 30%, 40%, 50%, 60%, 70%, 71%, 72%, 73%, 74%, 75%, 76%, 77%, 78%, 79%, 80%, 81%-84%, 85%, 86%, 87%, 88%, 89%, 90%, 91%, 92%, 93%, 94%, 95%, 96%, 97%, 98% or 99% nucleotide “sequence identity” to the nucleotide sequence of the wild type gene. Correspondingly, where an “allele” refers to the biochemical information for expressing a peptide or polypeptide, the respective nucleic acid sequence of the allele has at least, in increasing order of preference, 30%, 40%, 50%, 60%, 70%, 71%, 72%, 73%, 74%, 75%, 76%, 77%, 78%, 79%, 80%, 81%-84%, 85%, 86%, 87%, 88%, 89%, 90%, 91%, 92%, 93%, 94%, 95%, 96%, 97%, 98% or 99% amino acid “sequence identity” to the respective wild type peptide or polypeptide.

Mutations or alterations of amino or nucleic acid sequences can be any of substitutions, deletions or insertions; the terms “mutations” or “alterations” also encompass any combination of these. Hereinafter, all three specific ways of mutating are described in more detail by way of reference to amino acid sequence mutations; the corresponding teaching applies to nucleic acid sequences such that “amino acid” is replaced by “nucleotide”.

“Substitutions” are described by providing the original amino acid followed by the number of the position within the amino acid sequence, followed by the substituted amino acid. For example, the substitution of histidine at position 120 with alanine is designated as “His120Ala” or “H120A”.

“Deletions” are described by providing the original amino acid followed by the number of the position within the amino acid sequence, followed by “*” or “−”. Accordingly, the deletion of glycine at position 150 is designated as “Gly150*”, “G150*”, “Gly150-” or “G150-”. Alternatively, deletions are indicated by e.g. “deletion of D183 and G184”.

“Insertions” are described by providing the original amino acid followed by the number of the position within the amino acid sequence, followed by the original amino acid and the additional amino acid. For example, an insertion at position 180 of lysine next to glycine would be designated as “Gly180GlyLys” or “G180GK”. When more than one amino acid residue is inserted, such as e.g. a Lys and Ala after Gly180 this may be indicated as: Gly180GlyLysAla or G180GKA. In cases where a substitution and an insertion occur at the same position, this may be indicated as S99SD+S99A or in short S99AD. In cases where an amino acid residue identical to the existing amino acid residue is inserted, it is clear that degeneracy in the nomenclature arises. If for example a glycine is inserted after the glycine in the above example this would be indicated by G180GG.

Variants comprising multiple alterations are separated by “+”, e.g. “Arg170Tyr+Gly195Glu” or “R170Y+G195E” representing a substitution of arginine and glycine at positions 170 and 195 with tyrosine and glutamic acid, respectively. Alternatively, multiple alterations may be separated by space or a comma e.g. R170Y G195E or R170Y, G195E respectively.

Where different alterations can be introduced at a position, the different alterations are separated by a comma, e.g. “Arg170Tyr, Glu” represents a substitution of arginine at position 170 with tyrosine or glutamic acid. Alternatively, different alterations or optional substitutions may be indicated in brackets e.g. Arg170[Tyr, Gly] or Arg170{Tyr, Gly} or in short R170[Y,G] or R170{Y, G}.

This way, the invention provides Lr67 alleles of a soybean Lr67 gene, wherein each allele comprises, in the numbering according to SEQ ID NO. 1, at least one of the mutations G145R, G145K, G145H, G145Q, G145E, G145V, G145L, G145Y, G145R+1389L, G145K+1389L, G145H+1389L, G145Q+1389L, G145E+1389L, G145V+1389L, G145L+1389L, G145Y+1389L, G145R+1389W, G145K+1389W, G145H+1389W, G145Q+1389W, G145E+1389W, G145V+1389W, G145L+1389W, G145Y+1389W, G145R+1389K, G145K+1389K, G145H+1389K, G145Q+1389K, G145E+1389K, G145V+1389K, G145L+1389K, G145Y+1389K, G145R+1389R, G145K+1389R, G145H+1389R, G145Q+1389R, G145E+1389R, G145V+1389R, G145L+1389R, G145Y+1389R, G145R+1389Q, G145K+1389Q, G145H+1389Q, G145Q+1389Q, G145E+1389Q, G145V+1389Q, G145L+1389Q, G145Y+1389Q, G145R+1389F, G145K+1389F, G145H+1389F, G145Q+1389F, G145E+1389F, G145V+1389F, G145L+1389F, G145Y+1389F, G145R+1389M, G145K+1389M, G145H+1389M, G145Q+1389M, G145E+1389M, G145V+1389M, G145L+1389M and G145Y+1389M. Preferred Lr67 alleles according to the present invention are described herein as SEQ ID NO. 8, 9, 10, 11, 12, 13 and 14.

A special aspect concerning amino acid substitutions are conservative mutations which often appear to have a minimal effect on protein folding resulting in substantially maintained peptide or polypeptide properties of the respective peptide or polypeptide variant compared to the peptide or polypeptide properties of the parent peptide or polypeptide. Conservative mutations are those where one amino acid is exchanged with a similar amino acid. For determination of %-similarity the following applies, which is also in accordance with the BLOSUM62 matrix, which is one of the most used amino acids similarity matrix for database searching and sequence alignments:

Amino acid A is similar to amino acids S

Amino acid D is similar to amino acids E, N

Amino acid E is similar to amino acids D, K and Q

Amino acid F is similar to amino acids W, Y

Amino acid H is similar to amino acids N, Y

Amino acid I is similar to amino acids L, M and V

Amino acid K is similar to amino acids E, Q and R

Amino acid L is similar to amino acids I, M and V

Amino acid M is similar to amino acids I, L and V

Amino acid N is similar to amino acids D, H and S

Amino acid Q is similar to amino acids E, K and R

Amino acid R is similar to amino acids K and Q

Amino acid S is similar to amino acids A, N and T

Amino acid T is similar to amino acids S

Amino acid V is similar to amino acids I, L and M

Amino acid W is similar to amino acids F and Y

Amino acid Y is similar to amino acids F, H and W

Conservative amino acid substitutions may occur over the full length of the sequence of a polypeptide sequence of a functional protein such as a peptide or polypeptide. Preferably such mutations are not pertaining the functional domains of a peptide or polypeptide. Preferably, the amino acid sequence of the Lr67 allele of the present invention differs, after alignment, from a sequence selected from SEQ ID NO. 1, 2, 3, 4, 5, 6 and 7 only by an amino acid as provided by table 1. To use the table on a target sequence in question, the target sequence is first aligned to SEQ ID NO. 1 regardless of whether the target sequence has greater sequence identity to any other sequences selected from SEQ ID NO. 2, 3, 4, 5, 6 and 7. The numbering of positions is then according to SEQ ID NO. 1 regardless of insertions and deletions in the target sequence relative to SEQ ID NO. 1.

TABLE 1
List of preferred amino acids per position in the
sequence according to SEQ ID NO. 1. Amino acids
are given in decreasing order of preference. A
list of amino acids in brackets indicates that
the listed amino acids are equally preferred.
“-” denotes that the position can be deleted,
“X” indicates that any amino acid can be
located at the respective position.

| position | preferred amino acids |
|---|---|
| 1 | M |
| 2 | A; P; S; G |
| 3 | G; A; S; V |
| 4 | G; A; S; V |
| 5 | G; A; [SN] |
| 6 | F; Y; W; G |
| 7 | T; [IV]; [-S] |
| 8 | N; S; T; [-D] |
| 9 | A; T; S; [IV] |
| 10 | G; A; S; [TN] |
| 11 | G; [AS]; N |
| 12 | G; A; S; [QE] |
| 13 | G; A; S; K |
| 14 | D; E; Q; [HN] |
| 15 | F; Y; W |
| 16 | E; Q; D; [-PA] |
| 17 | A; G; S |
| 18 | K; R; Q; [AST] |
| 19 | I; V; L; M |
| 20 | T; S; [AN] |
| 21 | P; F; L; Y |
| 22 | I; V; [FL]; [YA] |
| 23 | V; I; [ML] |
| 24 | I; V; M; T |
| 25 | I; V; L; M |
| 26 | S; T; A |
| 27 | C; A; [MLIVST] |
| 28 | I; V; M; L |
| 29 | M; L; I; V |
| 30 | A; S; [GT] |
| 31 | A; S; [GT] |
| 32 | T; M; S; L |
| 33 | G; A; [SN] |
| 34 | G; A; [SN] |
| 35 | L; M; I |
| 36 | M; [LI]; V |
| 37 | F; Y; [WL] |
| 38 | G; A; [SN] |
| 39 | Y; F; W |
| 40 | D; [NE] |
| 41 | I; V; [ML] |
| 42 | G; A; [SN] |
| 43 | V; I; [ML]; [-A] |
| 44 | S; [AT] |
| 45 | G; A; [SN] |
| 46 | G; A; [SN] |
| 47 | V; I; [ML] |
| 48 | T; S; [AN] |
| 49 | S; [AT] |
| 50 | M; L; I |
| 51 | P; [AD]; V |
| 52 | P; A; S; V |
| 53 | F; Y; [WL] |
| 54 | L; M; I |
| 55 | K; R; Q; [DE] |
| 56 | K; R; Q; C |
| 57 | F; Y; [WL] |
| 58 | F; Y; [WL] |
| 59 | P; A; [STKQE] |
| 60 | T; E; S; [QD] |
| 61 | V; I; [ML] |
| 62 | Y; F; W; H |
| 63 | R; K; Q; [WH] |
| 64 | K; R; Q; [CD] |
| 65 | T; S; K; I |
| 66 | V; I; M; L |
| 67 | E; Q; D; [AST] |
| 68 | E; D; Q; V |
| 69 | K; [RQ]; [YHE] |
| 70 | G; A; S |
| 71 | L; [IV] |
| 72 | D; N; E; G |
| 73 | S; [AT] |
| 74 | N; Q; D; [ME] |
| 75 | Y; F; W |
| 76 | C; A; [MLIVST] |
| 77 | K; Q; R; [WE] |
| 78 | Y; F; W |
| 79 | D; [NE] |
| 80 | N; S; D; [VAC] |
| 81 | Q; E; K; [PIASTD] |
| 82 | G; T; A; [IV] |
| 83 | L; M; I |
| 84 | Q; E; K; [ICT] |
| 85 | L; M; I; [GQD] |
| 86 | F; Y; [WL] |
| 87 | T; S; [AN] |
| 88 | S; [AT] |
| 89 | S; A; T; [LIC] |
| 90 | L; M; I |
| 91 | Y; F; W |
| 92 | L; M; I |
| 93 | A; S; [GT] |
| 94 | G; A; S; [LIV] |
| 95 | L; M; I |
| 96 | T; M; [IV]; L |
| 97 | S; A; T; [ND] |
| 98 | T; S; A; [GRD] |
| 99 | F; Y; L; [MQ] |
| 100 | F; Y; L; [IV] |
| 101 | A; S; [GT] |
| 102 | S; A; T; [PLIVGC] |
| 103 | Y; H; F; [STE] |
| 104 | T; V; I; L |
| 105 | T; S; [AN] |
| 106 | R; K; Q |
| 107 | R; K; Q; [ICT] |
| 108 | L; M; F; [HQE] |
| 109 | G; A; [SN] |
| 110 | R; K; Q |
| 111 | R; K; Q; [CD] |
| 112 | L; M; I; [GQE] |
| 113 | T; S; A; [GRD] |
| 114 | M; L; I |
| 115 | L; M; I |
| 116 | I; V; L; [WYF] |
| 117 | A; S; G; [ND] |
| 118 | G; A; [SN] |
| 119 | F; [LI]; [WY] |
| 120 | F; Y; L; [IV] |
| 121 | F; Y; [WL] |
| 122 | I; V; L; [WFKRQ] |
| 123 | A; G; V; C |
| 124 | G; A; [SN] |
| 125 | V; I; T; [AGN] |
| 126 | V; I; L; [ASKQ] |
| 127 | L; [MI] |
| 128 | N; D; [STHKQ] |
| 129 | A; G; S; [LIV] |
| 130 | A; [VS]; [YFI] |
| 131 | A; S; [GT] |
| 132 | Q; E; N; [GD] |
| 133 | D; N; [SHE]; X |
| 134 | L; M; I; [PVATE] |
| 135 | A; S; [LT]; [WYF] |
| 136 | M; L; I; [YFAC] |
| 137 | L; M; I |
| 138 | I; V; [ML] |
| 139 | V; I; [ML]; [WPKRQE] |
| 140 | G; A; [SN] |
| 141 | R; K; Q |
| 142 | I; V; [ML]; [AS] |
| 143 | L; M; I |
| 144 | L; M; I |
| 145 | R; K; [HQE]; [VLY] |
| 146 | C; [AS]; [YFE] |
| 147 | G; A; [SN] |
| 148 | V; I; [ML]; [YF] |
| 149 | G; A; [SN] |
| 150 | F; Y; [WL] |
| 151 | A; S; [GT] |
| 152 | N; D; [STHKQ] |
| 153 | Q; E; [HKR] |
| 154 | A; S; T; [ND] |
| 155 | V; I; [ML] |
| 156 | P; A; [STKQE] |
| 157 | V; I; L; [PATE] |
| 158 | F; Y; W; H |
| 159 | L; M; I; [VT] |
| 160 | S; [AT] |
| 161 | E; Q; D |
| 162 | I; V; M; [WKRQ] |
| 163 | A; S; [GT] |
| 164 | P; A; [STKQE] |
| 165 | S; T; A; [YFG] |
| 166 | R; K; Q; [CD] |
| 167 | I; V; [ML]; [WYH] |
| 168 | R; K; Q |
| 169 | G; A; [SN] |
| 170 | A; S; [GT]; [LIV] |
| 171 | L; M; I |
| 172 | N; D; [STHKQ] |
| 173 | I; V; [ML] |
| 174 | L; M; I; [GND] |
| 175 | F; Y; [WL] |
| 176 | Q; E; [HKR] |
| 177 | L; M; I |
| 178 | N; S; D; [VAC] |
| 179 | [IV]; [ML]; [YF] |
| 180 | T; S; [AN] |
| 181 | I; V; L; [WKRQ] |
| 182 | G; A; [SN] |
| 183 | I; V; [ML] |
| 184 | L; M; I |
| 185 | F; Y; I [VT] |
| 186 | A; S; T; [ND] |
| 187 | N; D; [STHKQ] |
| 188 | L; I; M; [VT] |
| 189 | V; I; L; [FM] |
| 190 | N; D; [STHKQ] |
| 191 | Y; F; W |
| 192 | G; A; F; [YL] |
| 193 | T; S; A; [WYF] |
| 194 | N; [AS]; [HD] |
| 195 | K; R; Q |
| 196 | I; V; [ML] |
| 197 | K; R; Q; E |
| 198 | G; A; [SN] |
| 199 | G; A; [SN] |
| 200 | W; Y; F; [HD] |
| 201 | G; A; [SN] |
| 202 | W; Y; F |
| 203 | R; K; Q |
| 204 | L; M; I; [VT] |
| 205 | S; [AT] |
| 206 | L; M; I; [PVATE] |
| 207 | G; A; S; W |
| 208 | L; M; I; [GND] |
| 209 | A; G; S; [ND] |
| 210 | G; A; S; [MLI] |
| 211 | I; V; L; [WKRQ] |
| 212 | P; A; [STKQE] |
| 213 | A; S; [GT] |
| 214 | V; I; L |
| 215 | L; M; I; V |
| 216 | L; M; I; [VT] |
| 217 | T; S; [AN] |
| 218 | L; M; I; [WYF] |
| 219 | G; A; [SN] |
| 220 | A; S; T; [ND] |
| 221 | L; M; F; [WY] |
| 222 | F; L; [YI]; V |
| 223 | V; I; L; [FM] |
| 224 | V; I; M; [PH] |
| 225 | D; [NE] |
| 226 | T; S; [AN] |
| 227 | P; A; [STKQE] |
| 228 | N; D; [STHKQ] |
| 229 | S; [AT] |
| 230 | L; M; I; [GQD] |
| 231 | I; V; [ML] |
| 232 | E; Q; D |
| 233 | R; K; Q |
| 234 | G; A; [SN] |
| 235 | R; H; K; [MLD] |
| 236 | L; M; I; [RQE] |
| 237 | E; D; Q; [GN] |
| 238 | E; Q; K; [CR] |
| 239 | G; A; S; [LIV] |
| 240 | K; R; Q |
| 241 | T; A; S; [ND] |
| 242 | V; I; M; [HQE] |
| 243 | L; M; I; |
| 244 | K; R; Q; [DE] |
| 245 | K; R; Q; [ATD] |
| 246 | I; V; [ML] |
| 247 | R; K; Q |
| 248 | G; A; [SN] |
| 249 | T; I; V; L |
| 250 | D; E; N; [ARQ] |
| 251 | N; D; [STHKQ] |
| 252 | I; V; [ML]; [YK] |
| 253 | E; D; Q; [GN] |
| 254 | L; P; M; I |
| 255 | E; Q; D |
| 256 | F; Y; [WL] |
| 257 | Q; [NE]; [ML] |
| 258 | E; D; Q; [GN] |
| 259 | L; M; I; [WFKRQ] |
| 260 | [LV]; I; [FM] |
| 261 | E; D; Q; [FGC] |
| 262 | A; S; [GT] |
| 263 | S; [AT] |
| 264 | R; K; Q; [ATDE] |
| 265 | V; I; M; [SKRQE] |
| 266 | A; S; T; [ND] |
| 267 | K; R; Q; [GS] |
| 268 | E; Q; [AKD]; [MC] |
| 269 | V; I; [ML] |
| 270 | K; R; Q; [DE] |
| 271 | H; N; Q; [GD] |
| 272 | P; A; [STKQE] |
| 273 | F; Y; W; [GQ] |
| 274 | R; Q; K; [WMCDE] |
| 275 | N; D; [STHKQ] |
| 276 | L; M; I; [VT] |
| 277 | L; M; I |
| 278 | K; Q; R; G |
| 279 | R; K; Q |
| 280 | R; K; [HQ]; D |
| 281 | N; [HD]; [WYF] |
| 282 | R; K; Q |
| 283 | P; A; [STKQE] |
| 284 | Q; H; E; [YP] |
| 285 | L; M; I |
| 286 | V; I; T; [ND] |
| 287 | I; V; M; [WKRQ] |
| 288 | S; A; T; [ND] |
| 289 | [IV]; [ML]; [YF] |
| 290 | A; M; [LS]; G |
| 291 | L; M; I; V |
| 292 | Q; E; K; [FPIACST] |
| 293 | I; V; L; [WYF] |
| 294 | F; Y; [WL] |
| 295 | Q; E; [HKR] |
| 296 | Q; E; [HKR] |
| 297 | F; Y; L; [MI] |
| 298 | T; S; [AN] |
| 299 | G; A; [SN] |
| 300 | I; V; [ML] |
| 301 | N; D; [STHKQ] |
| 302 | A; S; V; [MLI] |
| 303 | I; V; [ML] |
| 304 | M; L; I |
| 305 | F; Y; [WL] |
| 306 | Y; F; W |
| 307 | A; S; [GT] |
| 308 | P; A; [STKQE] |
| 309 | V; I; [ML] |
| 310 | L; M; I |
| 311 | F; Y; [WL] |
| 312 | N; S; [TD]; [VAC] |
| 313 | T; S; A; [GRD] |
| 314 | L; M; I; [VT] |
| 315 | G; A; [SN] |
| 316 | F; Y; [WL] |
| 317 | K; R; Q |
| 318 | N; D; S; H |
| 319 | D; [NE] |
| 320 | A; S; [GT] |
| 321 | S; A; T; [PLIVGC] |
| 322 | L; M; I |
| 323 | Y; F; W; [MLI] |
| 324 | S; [AT] |
| 325 | A; S; [GT] |
| 326 | V; I; [ML] |
| 327 | I; V; [ML] |
| 328 | T; S; [AN] |
| 329 | G; A; [SN] |
| 330 | A; S; V; [MLI] |
| 331 | V; I; [ML] |
| 332 | N; D; [STHKQ] |
| 333 | V; I; [ML] |
| 334 | L; [IV]; [AT] |
| 335 | S; A; T; [ND] |
| 336 | T; S; [AN] |
| 337 | V; I; L; C |
| 338 | V; I; [ML] |
| 339 | S; [AT] |
| 340 | I; V; [ML] |
| 341 | Y; F; W |
| 342 | S; A; T; [WYF] |
| 343 | V; I; [ML] |
| 344 | D; [NE] |
| 345 | K; R; Q; [CD] |
| 346 | L; M; V; [WF] |
| 347 | G; A; [SN] |
| 348 | R; K; Q |
| 349 | R; K; Q; [CD] |
| 350 | M; [LI] |
| 351 | L; M; I; |
| 352 | L; M; F; [WY] |
| 353 | L; M; I |
| 354 | E; Q; D |
| 355 | A; G; S; [ND] |
| 356 | G; A; S; [LIVC] |
| 357 | V; I; A; [GS] |
| 358 | Q; E; [HKR] |
| 359 | M; L; I |
| 360 | F; Y; L; [MI] |
| 361 | L; I; M; [VT] |
| 362 | S; A; T; [LIC] |
| 363 | Q; E; [HKR] |
| 364 | V; I; M; [GQ] |
| 365 | V; I; [ML] |
| 366 | I; V; [ML]; [AS] |
| 367 | A; G; S; [ND] |
| 368 | I; V; T; [GN] |
| 369 | I; V; [ML]; [YG] |
| 370 | L; I; M; [VT] |
| 371 | G; A; [SN] |
| 372 | I; M; [LV]; [AGQ] |
| 373 | K; R; Q |
| 374 | V; I; L; [WYF] |
| 375 | T; [KQ]; G |
| 376 | D; N; E; [IVT] |
| 377 | H; Q; [YN]; D |
| 378 | S; A; T; [GN] |
| 379 | D; N; E; [VARQ] |
| 380 | D; N; E; S |
| 381 | L; M; I |
| 382 | S; N; T; V |
| 383 | K; R; Q; C |
| 384 | G; A; [WS]; [YF] |
| 385 | I; F; L; V |
| 386 | A; G; S; [ND] |
| 387 | I; V; N; D |
| 388 | L; M; I; V |
| 389 | L; [WKR]; [QFM] |
| 390 | V; I; [ML] |
| 391 | V; I; L; [FM] |
| 392 | M; L; I; [YF] |
| 393 | V; I; [ML]; [YF] |
| 394 | C; A; [MLIVST] |
| 395 | T; I; V; L |
| 396 | F; Y; W; [MLI] |
| 397 | V; I; [ML] |
| 398 | S; A; T; [ND] |
| 399 | S; A; T; [PLIVC] |
| 400 | F; Y; [WL] |
| 401 | A; S; [GT] |
| 402 | W; Y; F |
| 403 | S; [AT] |
| 404 | W; Y; F |
| 405 | G; A; [SN] |
| 406 | P; A; [STKQE] |
| 407 | L; M; I |
| 408 | G; A; S; [VT] |
| 409 | W; Y; F |
| 410 | L; M; I |
| 411 | I; V; [ML]; [AS] |
| 412 | P; A; [STKQE] |
| 413 | S; [AT] |
| 414 | E; Q; D |
| 415 | T; I; V; L |
| 416 | F; Y; [WL] |
| 417 | P; A; [STKQE] |
| 418 | L; M; I |
| 419 | E; Q; D |
| 420 | T; A; S; [LIV] |
| 421 | R; K; Q |
| 422 | S; [AT] |
| 423 | A; S; [GT] |
| 424 | G; A; S; [LIV] |
| 425 | Q; E; [HKR] |
| 426 | S; [AT] |
| 427 | V; I; [ML]; [YF] |
| 428 | T; [SN]; [AG] |
| 429 | V; I; [ML] |
| 430 | C; [AS]; [YFE] |
| 431 | V; I; T; [ND] |
| 432 | N; D; [STHKQ] |
| 433 | L; M; I; [GQD] |
| 434 | L; M; I |
| 435 | F; Y; L; [ACST] |
| 436 | T; S; [AN] |
| 437 | F; Y; [WL] |
| 438 | V; I; L; T |
| 439 | I; V; [ML] |
| 440 | A; S; [GT] |
| 441 | Q; E; [HKR] |
| 442 | A; [GS]; [IV] |
| 443 | F; Y; [WL] |
| 444 | L; M; I |
| 445 | S; T; A; [YMLIV] |
| 446 | M; L; I |
| 447 | L; M; I; [GQD] |
| 448 | C; A; [MLIVST] |
| 449 | H; Y; Q; [FL] |
| 450 | F; Y; L; W |
| 451 | K; R; Q |
| 452 | F; Y; [WL]; [MI] |
| 453 | G; A; [SN]; D |
| 454 | I; V; L; [WFKRQ] |
| 455 | F; Y; [WL] |
| 456 | L; M; F |
| 457 | F; Y; [WL] |
| 458 | F; Y; [WL] |
| 459 | S; A; T; [PLIVGC] |
| 460 | G; A; S; [WN] |
| 461 | W; Y; F; [IAC] |
| 462 | V; I; [ML] |
| 463 | L; M; I; F |
| 464 | V; I; [ML]; [YAG] |
| 465 | M; L; I |
| 466 | S; T; A; I |
| 467 | V; I; [MLT]; [YHN] |
| 468 | F; Y; [WL] |
| 469 | V; I; T; [ND] |
| 470 | L; M; I; [WYH] |
| 471 | F; L; Y; [MV] |
| 472 | L; M; F; V |
| 473 | L; M; I; [VT] |
| 474 | P; A; [STKQE] |
| 475 | E; Q; D |
| 476 | T; S; [AN] |
| 477 | K; R; Q |
| 478 | N; D; G; [WPA] |
| 479 | V; I; [ML]; [WPAKRQE] |
| 480 | P; A; [STKQE] |
| 481 | I; V; [ML] |
| 482 | E; Q; D |
| 483 | E; Q; D |
| 484 | M; L; I |
| 485 | T; S; [AN] |
| 486 | E; D; Q; [WAG] |
| 487 | R; K; Q; [WH] |
| 488 | V; I; [ML] |
| 489 | W; Y; F |
| 490 | K; R; Q; W |
| 491 | Q; E; N; [AG] |
| 492 | H; [YQ] |
| 493 | W; Y; F; [PAD] |
| 494 | F; Y; L; [MI] |
| 495 | W; Y; F |
| 496 | K; N; R; [ST] |
| 497 | R; K; Q; [ACST] |
| 498 | F; Y; W; H |
| 499 | I; V; M; A |
| 500 | D; E; N; [VQ] |
| 501 | D; E; N; [WYHR] |
| 502 | A; [-S]; [GT] |
| 503 | A; S; [GTDE] |
| 504 | D; [NE] |
| 505 | E; D; Q; [AC] |
| 506 | K; R; Q; [IV] |
| 507 | V; I; A; L |
| 508 | A; [SE]; G |
| 509 | N; H; [QE]; G |
| 510 | V; I; M; [KR] |
| 511 | S; [AT]; E |
| 512 | N; [MT]; [IV] |
| 513 | G; A; S; T |
| 514 | N; K; G; [THD] |
| 515 | G; N; A |
| 516 | F; Y; L; W |
| 517 | D; [-NE] |
| 518 | P; -; A; [STKQE] |
| 519 | T; I; [VS] |
| 520 | S; K; [AT]; - |
| 521 | R; K; Q |
| 522 | L; [MI]; - |

Preferably the amino acid sequence of the Lr67 allele of the present invention differs only by at most 80 positions, even more preferably by at most 50 positions, even more preferably by at most 30 positions, even more preferably by at most 25 positions, even more preferably by at most 20 positions, even more preferably by at most 19 positions, even more preferably by at most 18 positions, even more preferably by at most 17 positions, even more preferably by at most 16 positions, even more preferably by at most 15 positions, even more preferably by at most 14 positions, even more preferably by at most 13 positions, even more preferably by at most 12 positions, even more preferably by at most 11 positions, even more preferably by at most 10 positions, even more preferably by at most 9 positions, even more preferably by at most 8 positions, even more preferably by at most 7 positions, even more preferably by at most 6 positions, even more preferably by at most 5 positions, even more preferably by at most 4 positions, even more preferably by at most 3 positions, even more preferably by at most 2 positions, even more preferably by at most 1 position, after alignment, from one of the sequences SEQ ID NO. 1, 2, 3, 4, 5, 6 and 7. This way the functionality of the Lr67 allele of the present invention is best conserved even in those cases where the overall sequence of the Lr67 allele of the present invention has to be modified, for example to remove restriction sites that cannot be removed by a silent mutation.

It is particularly preferred that the amino acid sequence of the Lr67 allele of the present invention does not comprise an insertion compared to SEQ ID NO. 1. However, if an insertion is envisaged, then it is preferred that the insertion is only immediately after any of the following positions according to SEQ ID NO. 1: 10, 235, 249, 268, 287, 321 and/or 374.

Preferably, the amino acid sequence of the Lr67 allele of the present invention comprises, at the following positions according to SEQ ID NO. 1, only an amino acid as found at the respective position in one of the sequences SEQ ID NO. 1, 2, 3, 4, 5, 6 and 7, wherein for the sake of reference the respective amino acid according to SEQ ID NO. 1 is indicated: T20, V23, C27, A30, A31, G33, G34, L35, F37, G38, Y39, D40, G42, S44, G45, G46, V47, T48, M50, F53, L54, F57, F58, P59, V61, Y75, C76, K77, D79, Q81, L83, F86, T87, S88, S89, L90, Y91, A93, L95, A101, S102, T105, R106, G109, R110, L115, G118, F121, G124, A131, M136, L137, 1138, G140, R141, L144, G147, G149, F150, N152, Q153, V155, P156, L159, S160, E161, A163, P164, R168, G169, N172, F175, Q176, G182, L184, A186, N190, Y191, T193, 1196, G201, W202, R203, S205, L206, P212, A213, T217, G219, T226, P227, N228, S229, R233, L243, R247, G248, E255, A262, S263, H271, P272, F273, R279, R282, P283, Q284, L285, F294, Q295, Q296, T298, G299, 1300, N301, 1303, F305, Y306, A307, P308, V309, L310, F311, T313, G315, L322, S324, G329, S339, D344, G347, R348, L351, L353, G356, Q358, M359, Q363, C394, F400, S403, W404, G405, P406, L407, W409, L410, P412, S413, E414, F416, P417, L418, E419, R421, S422, A423, G424, Q425, S426, V429, F437, A440, Q441, F443, L444, M446, L447, C448, K451, F455, F457, F458, W461, M465, F468, P474, E475, T476, K477, N478, P480, 1481, E482, M484, V488, W489, H492, W493, W495. This way the functionality of the Lr67 allele of the present invention is conserved even in those cases where the overall sequence of the Lr67 allele of the present invention has to be modified, for example to remove restriction sites that cannot be removed by a silent mutation.

More preferably, the amino acid sequence of the Lr67 allele of the present invention comprises, at the following positions according to SEQ ID NO. 1, only an amino acid as found at the respective position in one of the sequences SEQ ID NO. 1, 2, 3, 4, 5, 6 and 7, wherein for the sake of reference the respective amino acid according to SEQ ID NO. 1 is indicated: Ml, G4, G12, E16, A17, K18, 119, T20, P21, V23, S26, C27, M29, A30, A31, T32, G33, G34, L35, M36, F37, G38, Y39, D40, G42, S44, G45, G46, V47, T48, M50, F53, L54, F57, F58, P59, V61, S73, N74, Y75, C76, K77, Y78, D79, N80, Q81, L83, Q84, L85, F86, T87, S88, S89, L90, Y91, L92, A93, G94, L95, T98, F99, A101, S102, T104, T105, R106, G109, R110, T113, M114, L115, 1116, A117, G118, F120, F121, G124, N128, A131, M136, L137, 1138, G140, R141, L143, L144, C146, G147, V148, G149, F150, A151, N152, Q153, A154, V155, P156, F158, L159, S160, E161, 1162, A163, P164, R166, 1167, R168, G169, L171, N172, 1173, L174, F175, Q176, L177, N178, T180, 1181, G182, 1183, L184, F185, A186, N187, L188, N190, Y191, G192, T193, K195, 1196, W200, G201, W202, R203, S205, L206, L208, A209, G210, P212, A213, L216, T217, G219, V223, T226, P227, N228, S229, L230, 1231, E232, R233, G234, E238, G239, K240, L243, 1246, R247, G248, T249, E253, E255, F256, E261, A262, S263, R264, A266, V269, K270, H271, P272, F273, R274, N275, L276, R279, N281, R282, P283, Q284, L285, 1287, L291, Q292, F294, Q295, Q296, T298, G299, 1300, N301, A302, 1303, M304, F305, Y306, A307, P308, V309, L310, F311, T313, G315, F316, A320, L322, Y323, S324, A325, V326, 1327, T328, G329, A330, V331, N332, V333, S335, T336, V338, S339, Y341, V343, D344, G347, R348, R349, L351, L352, L353, E354, A355, G356, Q358, M359, S362, Q363, 1366, A367, L370, G371, K373, D376, L381, A386, V389, V390, C394, V397, F400, A401, W402, S403, W404, G405, P406, L407, G408, W409, L410, 1411, P412, S413, E414, F416, P417, L418, E419, T420, R421, S422, A423, G424, Q425, S426, T428, V429, C430, V431, N432, F435, T436, F437, A440, Q441, F443, L444, S445, M446, L447, C448, H449, K451, 1454, F455, F457, F458, W461, V462, M465, S466, F468, V469, F471, P474, E475, T476, K477, N478, P480, 1481, E482, E483, M484, T485, V488, W489, K490, H492, W493, W495, R497. This way the functionality of the Lr67 allele of the present invention is even better conserved even in those cases where the overall sequence of the Lr67 allele of the present invention has to be modified, for example to remove restriction sites that cannot be removed by a silent mutation.

Most preferably, the amino acid sequence of the Lr67 allele of the present invention comprises, at the following positions according to SEQ ID NO. 1, only an amino acid as found at the respective position in one of the sequences SEQ ID NO. 1, 2, 3, 4, 5, 6 and 7, wherein, for the sake of reference the respective amino acid according to SEQ ID NO. 1 is indicated: Ml, G4, G5, F6, G12, F15, E16, A17, K18, 119, T20, P21, 122, V23, 124, S26, C27, 128, M29, A30, A31, T32, G33, G34, L35, M36, F37, G38, Y39, D40, G42, S44, G45, G46, V47, T48, S49, M50, F53, L54, K56, F57, F58, P59, V61, V66, S73, N74, Y75, C76, K77, Y78, D79, N80, Q81, G82, L83, Q84, L85, F86, T87, S88, S89, L90, Y91, L92, A93, G94, L95, S97, T98, F99, F100, A101, S102, Y103, T104, T105, R106, L108, G109, R110, T113, M114, L115, 1116, A117, G118, F120, F121, 1122, G124, N128, A130, A131, Q132, L134, A135, M136, L137, 1138, G140, R141, 1142, L143, L144, C146, G147, V148, G149, F150, A151, N152, Q153, A154, V155, P156, V157, F158, L159, S160, E161, 1162, A163, P164, R166, 1167, R168, G169, L171, N172, 1173, L174, F175, Q176, L177, N178, 1179, T180, 1181, G182, 1183, L184, F185, A186, N187, L188, V189, N190, Y191, G192, T193, K195, 1196, W200, G201, W202, R203, L204, S205, L206, L208, A209, G210, P212, A213, L215, L216, T217, G219, A220, L221, V223, T226, P227, N228, S229, L230, 1231, E232, R233, G234, L236, E238, G239, K240, V242, L243, 1246, R247, G248, T249, D250, E253, L254, E255, F256, E258, L259, L260, E261, A262, S263, R264, A266, V269, K270, H271, P272, F273, R274, N275, L276, L277, R279, N281, R282, P283, Q284, L285, 1287, L291, Q292, 1293, F294, Q295, Q296, T298, G299, 1300, N301, A302, 1303, M304, F305, Y306, A307, P308, V309, L310, F311, T313, L314, G315, F316, D319, A320, L322, Y323, S324, A325, V326, 1327, T328, G329, A330, V331, N332, V333, S335, T336, V338, S339, Y341, S342, V343, D344, G347, R348, R349, L351, L352, L353, E354, A355, G356, V357, Q358, M359, F360, S362, Q363, V364, 1366, A367, L370, G371, K373, V374, D376, S378, L381, G384, A386, V389, V390, C394, T395, V397, F400, A401, W402, S403, W404, G405, P406, L407, G408, W409, L410, 1411, P412, S413, E414, T415, F416, P417, L418, E419, T420, R421, S422, A423, G424, Q425, S426, V427, T428, V429, C430, V431, N432, L433, L434, F435, T436, F437, 1439, A440, Q441, A442, F443, L444, S445, M446, L447, C448, H449, K451, G453, 1454, F455, F457, F458, S459, W461, V462, M465, S466, F468, V469, F471, L473, P474, E475, T476, K477, N478, P480, 1481, E482, E483, M484, T485, E486, V488, W489, K490, H492, W493, W495, K496, R497, F498, D500, G515. This way the functionality of the Lr67 allele of the present invention is best conserved even in those cases where the overall sequence of the Lr67 allele of the present invention has to be modified, for example to remove restriction sites that cannot be removed by a silent mutation.

Even more preferably, the amino acid sequence of the Lr67 allele of the present invention differs only by at most 80 positions, even more preferably by at most 50 positions, even more preferably by at most 30 positions, even more preferably by at most 25 positions, even more preferably by at most 20 positions, even more preferably by at most 19 positions, even more preferably by at most 18 positions, even more preferably by at most 17 positions, even more preferably by at most 16 positions, even more preferably by at most 15 positions, even more preferably by at most 14 positions, even more preferably by at most 13 positions, even more preferably by at most 12 positions, even more preferably by at most 11 positions, even more preferably by at most 10 positions, even more preferably by at most 9 positions, even more preferably by at most 8 positions, even more preferably by at most 7 positions, even more preferably by at most 6 positions, even more preferably by at most 5 positions, even more preferably by at most 4 positions, even more preferably by at most 3 positions, even more preferably by at most 2 positions, even more preferably by at most 1 position, after alignment, from a sequence selected from SEQ ID NO. 1, 2, 3, 4, 5, 6 and 7, wherein each difference is selected according to table 1 as described above, such that no insertions and deletions are allowed other than those indicated by Table 1. This way the functionality of the Lr67 allele of the present invention is best conserved even in those cases where the overall sequence of the Lr67 allele of the present invention has to be modified, for example to remove restriction sites that cannot be removed by a silent mutation.

Where an even closer adherence to a soy Lr67 gene sequence is desired, preferably the amino acid present at, in the numbering of SEQ ID NO. 1, at least one of the following positions is chosen from the respective amino acids of any of SEQ ID NO. 2, 3, 4, 5, 6 and 7: X68[ED], X157V, X170A, X278K and X380[DS], more preferably the amino acid is chosen from any of SEQ ID NO. 2, 3, 4, 5, 6 and 7at least at two of the respective positions, even more preferably at at least three of the respective positions, and most preferably at least four of the respective positions and most preferably at all of the respective positions.

If the skilled person selects to optimize the Lr67 allele of the present invention for a use in dicot plants, then preferably at least one of the sequence positions is conserved as follows: Y78, G239, K245, N251 and K345, more preferably at least two of these positions are conserved, more preferably at least three of these positions are conserved, more preferably at least four of these positions are conserved, most preferably all of these positions are conserved as indicated. In particular, the amino acid sequence of the Lr67 allele should not be that of any of the sequences SEQ ID NO. 1, 4, 7, 8 or 9 of WO2015024066 or a sequence differing from any of the aforementioned sequences only by (a) a substitution of G145R, G145L or G145H and/or (b) a substitution of 1389L, 13891, 1389M, 1389A or 1389F.

Protein or nucleic acid variants may be defined by their sequence identity when compared to a parent protein or nucleic acid. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

The following example is meant to illustrate two nucleotide sequences, but the same calculations apply to protein sequences:

Seq A: AAGATACTG length: 9 bases
Seq B: GATCTGA length: 7 bases

Hence, the shorter sequence is sequence B.

Producing a pairwise global alignment which is showing both sequences over their complete lengths results in

Seq A: AAGATACTG-
||| |||
Seq B: --GAT-CTGA

The “I” symbol in the alignment indicates identical residues (which means bases for DNA or amino acids for proteins). The number of identical residues is 6.

The “-” symbol in the alignment indicates gaps. The number of gaps introduced by alignment within the sequence B is 1. The number of gaps introduced by alignment at borders of sequence B is 2, and at borders of sequence A is 1.

The alignment length showing the aligned sequences over their complete length is 10.

Producing a pairwise alignment which is showing the shorter sequence over its complete length according to the invention consequently results in:

Seq A: GATACTG-
||| |||
Seq B: GAT-CTGA

Producing a pairwise alignment which is showing sequence A over its complete length according to the invention consequently results in:

Seq A: AAGATACTG
||| |||
Seq B: --GAT-CTG

Producing a pairwise alignment which is showing sequence B over its complete length according to the invention consequently results in:

Seq A: GATACTG-
||| |||
Seq B: GAT-CTGA

The alignment length showing the shorter sequence over its complete length is 8 (one gap is present which is factored in the alignment length of the shorter sequence).

Accordingly, the alignment length showing sequence A over its complete length would be 9 (meaning sequence A is the sequence of the invention), the alignment length showing sequence B over its complete length would be 8 (meaning sequence B is the sequence of the invention).

After aligning the two sequences, in a second step, an identity value shall be determined from the alignment. Therefore, according to the present description the following calculation of percent-identity applies:

%-identity=(identical residues/length of the alignment region which is showing the respective sequence of this invention over its complete length)*100. Thus, sequence identity in relation to comparison of two amino acid sequences according to the invention is calculated by dividing the number of identical residues by the length of the alignment region which is showing the respective sequence of this invention over its complete length. This value is multiplied with 100 to give “%-identity”. According to the example provided above, %-identity is: for sequence A being the sequence of the invention (6/9)*100=66.7%; for sequence B being the sequence of the invention (6/8)*100=75%.

Thus, the pairwise sequence identities of amino acid sequences SEQ ID NO. 1-7 are (percentages):

| global Identities | | | | | | | |
|---|---|---|---|---|---|---|---|
| by true pairwise | | | | | | | |
| alignments using | | | | | | | |
| program NEEDLE | | | | | | | |
| gapopen 10.0; | SEQ | SEQ | SEQ | SEQ | SEQ | SEQ | SEQ |
| gapextend 0.5; | ID | ID | ID | ID | ID | ID | ID |
| EBLOSUM62 | NO. 1 | NO. 2 | NO. 3 | NO. 4 | NO. 5 | NO. 6 | NO. 7 |
| SEQ ID NO. 1 | 100 | 97.9 | 98.7 | 90 | 77 | 82.4 | 72.6 |
| SEQ ID NO. 2 | 97.9 | 100 | 97.5 | 89.3 | 76.4 | 81 | 72.2 |
| SEQ ID NO. 3 | 98.7 | 97.5 | 100 | 90.6 | 76.2 | 81.2 | 71.8 |
| SEQ ID NO. 4 | 90 | 89.3 | 90.6 | 100 | 70.6 | 74 | 64.6 |
| SEQ ID NO. 5 | 77 | 76.4 | 76.2 | 70.6 | 100 | 89.5 | 65.5 |
| SEQ ID NO. 6 | 82.4 | 81 | 81.2 | 74 | 89.5 | 100 | 66.7 |
| SEQ ID NO. 7 | 72.6 | 72.2 | 71.8 | 64.6 | 65.5 | 66.7 | 100 |

The term “hybridisation” as defined herein is a process wherein substantially complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tm) for the specific sequence at a defined ionic strength and pH. Medium stringency conditions are when the temperature is 20° C. below Tm, and high stringency conditions are when the temperature is 10° C. below Tm. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore, medium stringency hybridisation conditions may sometimes be needed to identify such nucleic acid molecules.

The “Tm” is the temperature under defined ionic strength and pH, at which 50% of the target sequence hybridises to a perfectly matched probe. The Tm is dependent upon the solution conditions and the base composition and length of the probe. For example, longer sequences hybridise specifically at higher temperatures. The maximum rate of hybridisation is obtained from about 16° C. up to 32° C. below Tm. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tm may be calculated using the following equations, depending on the types of hybrids:
DNA-DNA hybrids (Meinkoth and Wahl, Anal. Biochem., 138: 267-284, 1984):

Tm=81.5° C. +16.6×log([Na+]{a})+0.41x %[G/C{b}]-500×[L{c}]-1-0.61x % formamide
DNA-RNA or RNA-RNA hybrids:

Tm=79.8+18.5 (log 10[Na+]{a})+0.58 (% G/C{b})+11.8 (% G/C{b})2-820/L{c}
oligo-DNA or oligo-RNAd hybrids:

for <20 nucleotides: Tm=2 ({1n})

for 20-35 nucleotides: Tm=22+1.46 ({1n})

wherein:

{a} or for other monovalent cation, but only accurate in the 0.01-0.4 M range

{b} only accurate for % GC in the 30% to 75% range

{c} L=length of duplex in base pairs

{d} Oligo, oligonucleotide

{In} effective length of primer=2×(no. of G/C)+(no. of A/T)

Non-specific binding may be controlled using any one of a number of known techniques such as, for example, blocking the membrane with protein containing solutions, additions of heterologous RNA, DNA, and SDS to the hybridisation buffer, and treatment with Rnase. For non-related probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCl and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate. Another example of high stringency conditions is hybridisation at 65° C. in 0.1×SSC comprising 0.1 SDS and optionally 5×Denhardt's reagent, 100 μg/ml denatured, fragmented salmon sperm DNA, 0.5% sodium pyrophosphate, followed by the washing at 65° C. in 0.3×SSC.

For the purposes of defining the level of stringency, reference can be made to Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

The invention in particular can be put into practice by applying a non-supervised, directed evolution system, preferably on a block-chain technology oriented disruptive agile innovation platform, which the skilled person will devise in the near future. By entrepreneurially applying bioinformatics techniques, the skilled person will materialize business opportunities in the field, thereby satisfying demands by farmers and breeders alike.

As used herein, the term “isolated DNA molecule” refers to a DNA molecule at least partially separated from other molecules normally associated with it in its native or natural state. The term “isolated” preferably refers to a DNA molecule that is at least partially separated from some of the nucleic acids which normally flank the DNA molecule in its native or natural state. Thus, DNA molecules fused to regulatory or coding sequences with which they are not normally associated, for example as the result of recombinant techniques, are considered isolated herein. Such molecules are considered isolated when integrated into the chromosome of a host cell or present in a nucleic acid solution with other DNA molecules, in that they are not in their native state.

Any number of methods well known to those skilled in the art can be used to isolate and manipulate a polynucleotide, or fragment thereof, as disclosed herein. For example, polymerase chain reaction (PCR) technology can be used to amplify a particular starting polynucleotide molecule and/or to produce variants of the original molecule. Polynucleotide molecules, or fragment thereof, can also be obtained by other techniques, such as by directly synthesizing the fragment by chemical means, as is commonly practiced by using an automated oligonucleotide synthesizer. A polynucleotide can be single-stranded (ss) or double-stranded (ds). “Double-stranded” refers to the base-pairing that occurs between sufficiently complementary, anti-parallel nucleic acid strands to form a double-stranded nucleic acid structure, generally under physiologically relevant conditions. Embodiments of the method include those wherein the polynucleotide is at least one selected from the group consisting of sense single-stranded DNA (ssDNA), sense single-stranded RNA (ssRNA), double-stranded RNA (dsRNA), double-stranded DNA (dsDNA), a double-stranded DNA/RNA hybrid, anti-sense ssDNA, or anti-sense ssRNA; a mixture of polynucleotides of any of these types can be used.

As used herein, “recombinant” when referring to nucleic acid or polypeptide, indicates that such material has been altered as a result of human application of a recombinant technique, such as by polynucleotide restriction and ligation, by polynucleotide overlap-extension, or by genomic insertion or transformation. A gene sequence open reading frame is recombinant if (a) that nucleotide sequence is present in a context other than its natural one, for example by virtue of being (i) cloned into any type of artificial nucleic acid vector or (ii) moved or copied to another location of the original genome, or if (b) the nucleotide sequence is mutagenized such that it differs from the wild type sequence. The term recombinant also can refer to an organism having a recombinant material, e.g., a plant that comprises a recombinant nucleic acid is a recombinant plant.

The term “transgenic” refers to an organism, preferably a plant or part thereof, or a nucleic acid that comprises a heterologous polynucleotide. Preferably, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to refer to any cell, cell line, callus, tissue, plant part or plant, the genotype of which has been so altered by the presence of heterologous nucleic acid including those transgenic organisms or cells initially so altered, as well as those created by crosses or asexual propagation from the initial transgenic organism or cell. A “recombinant” organism preferably is a “transgenic” organism. The term “transgenic” as used herein is not intended to encompass the alteration of the genome (chromosomal or extra-chromosomal) by conventional plant breeding methods (e.g., crosses) or by naturally occurring events such as, e.g., self-fertilization, random cross-fertilization, non-recombinant viral infection, non-recombinant bacterial transformation, non-recombinant transposition, or spontaneous mutation.

As used herein, “mutagenized” refers to an organism or nucleic acid thereof having alteration(s) in the biomolecular sequence of its native genetic material as compared to the sequence of the genetic material of a corresponding wildtype organism or nucleic acid, wherein the alteration(s) in genetic material were induced and/or selected by human action. Examples of human action that can be used to produce a mutagenized organism or DNA include, but are not limited to treatment with a chemical mutagen such as EMS and subsequent selection with herbicide(s); or by treatment of plant cells with x-rays and subsequent selection with herbicide(s). Any method known in the art can be used to induce mutations. Methods of inducing mutations can induce mutations in random positions in the genetic material or can induce mutations in specific locations in the genetic material (i.e., can be directed mutagenesis techniques), such as by use of a genoplasty technique. In addition to unspecific mutations, according to the invention a nucleic acid can also be mutagenized by using mutagenesis means with a preference or even specificity for a particular site, thereby creating an artificially induced heritable allele according to the present invention. Such means, for example site specific nucleases, including for example zinc finger nucleases (ZFNs), meganucleases, transcription activator-like effector nucelases (TALENS) (Malzahn et al., Cell Biosci, 2017, 7:21) and clustered regularly interspaced short palindromic repeats/CRISPR-associated nuclease (CRISPR/Cas) with an engineered crRNA/tracr RNA (for example as a single-guide RNA, or as modified crRNA and tracrRNA molecules which form a dual molecule guide), and methods of using this nucleases to target known genomic locations, are well-known in the art (see reviews by Bortesi and Fischer, 2015, Biotechnology Advances 33: 41-52; and by Chen and Gao, 2014, Plant Cell Rep 33: 575-583, and references within).

As used herein, a “genetically modified organism” (GMO) is an organism whose genetic characteristics contain alteration(s) that were produced by human effort causing transfection that results in transformation of a target organism with genetic material from another or “source” organism, or with synthetic or modified-native genetic material, or an organism that is a descendant thereof that retains the inserted genetic material. The source organism can be of a different type of organism (e.g., a GMO plant can contain bacterial genetic material) or from the same type of organism (e.g., a GMO plant can contain genetic material from another plant).

As used herein, “wildtype” or “corresponding wildtype plant” means the typical form of an organism or its genetic material, as it normally occurs, as distinguished from e.g. mutagenized and/or recombinant forms. Similarly, by “control cell” or “similar, wildtype, plant, plant tissue, plant cell or host cell” is intended a plant, plant tissue, plant cell, or host cell, respectively, that lacks the particular polynucleotide of the invention that are disclosed herein. The use of the term “wildtype” is not, therefore, intended to imply that a plant, plant tissue, plant cell, or other host cell lacks recombinant DNA in its genome, and/or does not possess fungal resistance characteristics that are different from those disclosed herein.

As used herein, “descendant” refers to any generation plant. A progeny or decendant plant can be from any filial generation, e.g., F1, F2, F3, F4, F5, F6, F7, etc. In some embodiments, a descendant or progeny plant is a first, second, third, fourth, fifth, sixth, seventh, eighth, ninth, or tenth generation plant.

The term “plant” is used herein in its broadest sense as it pertains to organic material and is intended to encompass eukaryotic organisms that are members of the taxonomic kingdom plantae, examples of which include but are not limited to monocotyledon and dicotyledon plants, vascular plants, vegetables, grains, flowers, trees, herbs, bushes, grasses, vines, ferns, mosses, fungi and algae, etc, as well as clones, offsets, and parts of plants used for asexual propagation (e.g. cuttings, pipings, shoots, rhizomes, underground stems, clumps, crowns, bulbs, corms, tubers, rhizomes, plants/tissues produced in tissue culture, etc.). Unless stated otherwise, the term “plant” refers to a whole plant, any part thereof, or a cell or tissue culture derived from a plant, comprising any of: whole plants, plant components or organs (e.g., leaves, stems, roots, etc.), plant tissues, seeds, plant cells, and/or progeny of the same. A plant cell is a biological cell of a plant, taken from a plant or derived through culture from a cell taken from a plant.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp., Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amaranth, artichoke, asparagus, broccoli, Brussels sprouts, cabbage, canola, carrot, cauliflower, celery, collard greens, flax, kale, lentil, oilseed rape, okra, onion, potato, rice, soybean, strawberry, sugar beet, sugar cane, sunflower, tomato, squash, tea and algae, amongst others. According to a preferred embodiment of the present invention, the plant is a crop plant. Examples of crop plants include inter alia soybean, sunflower, canola, alfalfa, rapeseed, cotton, tomato, potato or tobacco.

Preferably, the plant is or the plant part is derived from a monocotyledon or dicotyledon plant, more preferably a plant of order Fabales, more preferably a plant of family Fabaceae, more preferably a plant of tribus Phaseoleae, more preferably of genus Glycine, most preferably of species Glycine max, Glycine soja, Glycine gracilis or a cross Glycine max×Glycine soja.

The term “seed” comprises seeds of all types, such as, for example, true seeds, caryopses, achenes, fruits, tubers, seedlings and similar forms. Preferably “seed” refers to true seed(s) unless otherwise specified. For example, the seed can be seed of transgenic plants or plants obtained by site specific mutagenesis, by mutagenesis with a site preference or by traditional breeding methods. Examples of traditional breeding methods are cross-breeding, selfing, back-crossing, embryo rescue, in-crossing, out-crossing, inbreeding, selection, asexual propagation, and other traditional techniques as are known in the art.

According to the invention, a cell is provided having an Lr67 allele comprising one or more mutations in a soybean Lr67 gene, wherein the one or more mutations comprise, in the numbering according to SEQ ID NO. 1, a substitution at position G145, preferably a substitution selected from, in decreasing order of preference, G145R, G145K, G145H, G145Q, G145E, G145V, G145L and G145Y, and optionally also a substitution at position 1389, preferably a substitution selected from, in decreasing order of preference, 1389L, 1389W, 1389K, 1389R, 1389Q, 1389F and 1389M. Accordingly, the invention provides an Lr67 allele having, in increasing order of preference, at least 40%, 50%, 60%, 70%, 71%, 72%, 73%, 74%, 75%, 76%, 77%, 78%, 79%, 80%, 81%-84%, 85%, 86%, 87%, 88%, 89%, 90%, 91%, 92%, 93%, 94%, 95%, 96%, 97%, 98% or 99% amino acid sequence identity to SEQ ID NO. 1, as long as one of the following mutations, according to the amino acid numbering of SEQ ID NO. 1 and sorted in decreasing order of preference, are comprised therein: G145R, G145K, G145H, G145Q, G145E, G145V, G145L, G145Y. Preferably the Lr67 allele of the present invention comprises, in the numbering according to SEQ ID NO. 1, any of the following mutations: G145R+1389L, G145K+1389L, G145H+1389L, G145Q+1389L, G145E+1389L, G145V+1389L, G145L+1389L, G145Y+1389L, G145R+1389W, G145K+1389W, G145H+1389W, G145Q+1389W, G145E+1389W, G145V+1389W, G145L+1389W, G145Y+1389W, G145R+1389K, G145K+1389K, G145H+1389K, G145Q+1389K, G145E+1389K, G145V+1389K, G145L+1389K, G145Y+1389K, G145R+1389R, G145K+1389R, G145H+1389R, G145Q+1389R, G145E+1389R, G145V+1389R, G145L+1389R, G145Y+1389R, G145R+1389Q, G145K+1389Q, G145H+1389Q, G145Q+1389Q, G145E+1389Q, G145V+1389Q, G145L+1389Q, G145Y+1389Q, G145R+1389F, G145K+1389F, G145H+1389F, G145Q+1389F, G145E+1389F, G145V+1389F, G145L+1389F, G145Y+1389F, G145R+1389M, G145K+1389M, G145H+1389M, G145Q+1389M, G145E+1389M, G145V+1389M, G145L+1389M and G145Y+1389M.

As will be seen in the examples disclosed herein, such allele according to the invention, when expressed in a cell, enhances resistance of the cell to infections by at least one biotrophic or heminecrotrophic fungus. Furthermore, the allele, when expressed in a cell, provides a stable enhancement of the cell to such infections. Thus, the allele of the present invention is suitable for conferring, intensifying or stabilising resistance against fungal pathogen infections, particularly against biotrophic or heminecrotrophic fungi, and preferably against fungi as described below:

Preferably, the one or more mutations reduce or abolish, when expressing said Lr67 allele in a soybean plant, the susceptibility, relative to a wild type plant, to infections by a biotrophic or heminecrotrophic fungus, preferably a rust fungus, more preferably a fungus of phylum Basidiomycota, even more preferably of subphylum Pucciniomycotina, even more preferably of class Pucciniomycetes, even more preferably of order Pucciniales, even more preferably of family Chaconiaceae, Coleosporiaceae, Cronartiaceae, Melampsoraceae, Mikronegeriaceae, Phakopsoraceae, Phragmidiaceae, Pileolariaceae, Pucciniaceae, Pucciniastraceae, Pucciniosiraceae, Raveneliaceae, Sphaerophragmiaceae or Uropyxidaceae, even more preferably of genus Maravalia, Ochropsora, Olivea, Chrysomyxa, Coleosporium, Diaphanopellis, Cronartium, Endocronartium, Peridermium, Melampsora, Chrysocelis, Mikronegeria, Arthuria, Batistopsora, Cerotelium, Dasturella, Phakopsora, Prospodium, Arthuriomyces, Catenulopsora, Gerwasia, Gymnoconia, Hamaspora, Kuehneola, Phragmidium, Trachyspora, Triphragmium, Atelocauda, Pileolaria, Racospermyces, Uromycladium, Allodus, Ceratocoma, Chrysocyclus, Cumminsiella, Cystopsora, Endophyllum, Gymnosporangium, Miyagia, Puccinia, Puccorchidium, Roestelia, Sphenorchidium, Stereostratum, Uromyces, Hyalopsora, Melampsorella, Melampsoridium, Milesia, Milesina, Naohidemyces, Pucciniastrum, Thekopsora, Uredinopsis, Chardoniella, Dietelia, Pucciniosira, Diorchidium, Endoraecium, Kernkampella, Ravenelia, Sphenospora, Austropuccinia, Nyssopsora, Sphaerophragmium, Dasyspora, Leucotelium, Macruropyxis, Porotenus, Tranzschelia or Uropyxis, even more preferably of species Phakopsora pachyrhizi, Phakopsora meibomiae, Puccinia graminis, Puccinia striiformis, Puccinia hordei or Puccinia recondita and most preferably of species Phakopsora pachyrhizi.

This effect of the Lr67 allele of the present invention was all the more surprising given that a Lr67 allele of wheat did not provide any statistically significant enhancement to soybean resistance against Phakopsora pachyrhizi.

The cell according to the present invention containing the Lr67 allele of the present invention can be a yeast cell. As used herein the term “yeast” includes ascosporogenous yeast of taxonomic order Saccharomycetales, basidiosporogenous yeast and yeast belonging to the Fungi Imperfecti (Blastomycetes). Preferred yeast cells belong to any of the genera Candida, Hansenula, Kluyveromyces, Lachancea, Pichia, Saccharomyces, Schizosaccharomyces or Yarrowia and preferably belong to any of Kluyveromyces lactis, Lachancea kluyveri, Saccharomyces carlsbergensis, Saccharomyces cerevisiae, Saccharomyces cerevisiae var. diastaticus, Saccharomyces douglasii, Saccharomyces norbensis or Yarrowia lipolytica. The use of the Lr67 allele of the present invention advantageously allows to explore further variants or mutants of the Lr67 gene in a model organism having fast generation times and requiring only standard material and methods for handling and analysis. Thus, the invention provides a method for assaying a variant of an Lr67 allele of the present invention, comprising the steps of providing in a yeast cell an expression cassette for expression of an Lr67 allele of the present invention, exposing the nucleic acid coding for the Lr67 allele product to a mutagenizing agent and assaying the efficacy of the mutagenized allele for resistance to a biotrophic or heminecrotrophic fungus.

Preferably, the Lr67 gene
a) codes for a polypeptide having an amino acid sequence of at least 40%, more preferably at least 50%, more preferably at least 60%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%-84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98% or 99% identity to any of SEQ ID NO. 1, 2, 3, 4, 5, 6 or 7,
or a biologically active fragment thereof, and/or
b) comprises a nucleic acid sequence
   obtainable or obtained by amplification from the genome of Glycine max using any of the primer pairs according to SEQ ID NO. 15 and 21, 16 and 22, 17 and 23, 18 and 24, 19 and 25, or 20 and 26, and/or
   obtainable or obtained by reverse translation from an RNA hybridizing under stringent conditions to a nucleic acid probe according to SEQ ID NO. 27, 28, 29, 30, 31 or 32, and/or
   having at least 60%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%-84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98% or 99% identity to any of SEQ ID NO. 33, 34, 35, 36, 37 or 38, and/or
c) comprises a PF00083 Pfam domain, an InterPro domain selected from IPR020846, IPR005828, IPR036259, IPR003663 and IPR005829, a Prosite PS50850 Major facilitator superfamily (MFS) profile and/or 12 transmembrane domains.

Thus, the Lr67 allele of the Lr67 gene shares the above described properties of the Lr67 gene when leaving the substitutions at position 145 and/or 389 out of consideration.

It is understood that all references to the content of databases are to those contents as publicly available on 2019-01-15. Thus, if for example a sequence is updated in Uniprot after 2019-01-15, then the sequence as available at the beginning of 2019-01-15 CET is relevant.

According to the invention, the Lr67 allele preferably is a hypomorphic allele, an amorphic allele, a neomorphic allele or an antimorphic allele, preferably a dominant-negative allele. It is a particular advantage of the Lr67 allele of the present invention that enhancement in fungal resistance as described herein, in particular a statistically significant stable provision or intensification of resistance against biotrophic or heminecrotrophic fungi and most preferably against Phakopsora pachyrhizi can be achieved in polyploid plants, e.g. in Glycine max, already by merely incorporating the Lr67 allele into one set of chromosomes. This facilitates the generation, propagation and breeding of plants comprising the Lr67 allele of the present invention.

It is thus a particular advantage of the present invention that the cell according to the invention, when in meiosis, can be non-segregating or, less preferred, segregating for the Lr67 allele without abolishing the improved resistance conferred by the Lr67 allele of the present invention.

Further preferably according to the invention, the cell is homozygous for the Lr67 gene or, less preferred, heterozygous for the Lr67 gene. Whereas homozygous cells are particularly suitable for breeding, it is noteworthy that the advantages of the present invention are already conferred by cells that are heterozygous with regards to the Lr67 allele of the present invention. Thus, the present invention particularly facilitates the generation of hybrid plant varieties, i.e. first generation results of a crossing of parent plants wherein only one parent plant confers the Lr67 allele of the present invention, such that the hybrid plants already exhibit enhanced resistance to infections by at least one biotrophic or heminecrotrophic fungus. As preferred herein, such plants preferably are of genus Glycine, most preferably of species Glycine max, and the fungus in question preferably is of genus Phakopsora, most preferably of species Phakopsora pachyrhizi.

The Lr67 allele according to the present invention preferably codes for a polypeptide differing from any of SEQ ID NO. 1, 2, 3, 4, 5, 6 or 7 only
a) by one or more conservative amino acid substitutions, and/or
b) by one or more mutations according to table 1.

Such alleles generally leave the characteristic properties of the Lr67 gene unamended and thus, when compared to other mutations, improve the probability of maintaining the advantages conferred by the Lr67 allele of the present invention.

According to the present invention, the Lr67 allele is preferably integral part of the genome of the cell. This particularly facilitates the generation, propagation and breeding of plants comprising the Lr67 allele of the present invention. In particular, the Lr67 allele of the present invention can be integral to one or more of the cell's nuclear chromosomes and/or in the chloroplast and/or mitochondrial genetic material. Furthermore, the Lr67 allele of the present invention can be located other than in the genome of the cell, in particular in the form of plasmids. However, while this kind of integration into the cell of a plant is easy to accomplish there is an increased danger that the Lr67 allele is not handed down to offspring cells, in particular to seed cells.

Preferably, the Lr67 allele is operably linked to a heterologous promoter, preferably a rust-inducible promoter. This way the Lr67 allele is demonstrably in a man-made cell. The term “operably linked” means that the nucleic acid based elements, preferably a promoter, a transcription factor binding site or a coding sequence, are linked to one another in such a way that their function is coordinated and preferably allows the expression of the coding sequence. According to the invention the promoter is operably linked to the Lr67 allele, preferably such that both are contained within an expression cassette as described herein, such that the promoter is capable of expressing the Lr67 allele of the present invention in a cell, preferably a plant cell as described herein. Even more preferably, the promoter is plant tissue specific and/or pathogen-inducible.

Preferably, the cell contains a negligibly low content of, and preferably none of, allergens and/or anti-metabolites. This way the cell is particularly suitable for further processing into food or feed as described herein. It is thus particularly preferred that the cell is part of a plant such that the plant is a hypoallergenic plant. For purposes of the present invention, the term “hypoallergenic” means a decreased tendency to cause an allergic reaction through the substantial (i.e., greater than about 30%) reduction or complete (100%) elimination of activity of allergenic proteins. Where the cell is a cell of a plant of order Fabales, the plant or cell, respectively, should be negligibly low in content of any of the allergens
for genus Glycine, in particular for soybean: P34, Gly m Bd 28k, glycinin G3, KTI 1, Gly m2, Gly m IA, Gly m IB, rGLY m3, glycinin G1 and alpha-subunit of beta-conglycinin,
for genus Arachis, in particular for peanuts: Ara h1 to Ara h8.

Preferably, the plant or cell, respectively, is also negligibly low in anti-metabolite content. And even more preferably the plant is a high oleic acid plant.

It is a particular advantage of the present invention that the Lr67 allele of the present invention can be combined with any of the aforementioned further genes for conferring hypoallergenicity and/or low content of anti-nutrients. This was all the more surprising since it is known that in soybean gene silencing is a major factor contributing to spontaneous decrease of gene expression when two individually effective genes are combined, for example by crossing or co-transformation, into one plant's genetic material.

Preferably, the cell according to the present invention is a recombinant or transgenic plant cell. Also preferably the mutation in the Lr67 gene is an artificially induced heritable allele. Means for the generation of such artificially induced heritable alleles are described above. Either way the cell according to the preferred invention comprises the Lr67 allele stably integrated into its genome, thereby improving stability of the resistance effect when growing a field of plants under fungal pathogen exposure as listed herein, preferably under exposure to a fungus of genus Phakopsora, most preferably under exposure to a fungus of species Phakopsora pachyrhizi.

The invention correspondingly also provides an expression construct comprising an Lr67 allele comprising one or more mutations in a soybean Lr67 gene as defined herein, wherein the Lr67 allele is operably linked to a heterologous polynucleotide. Such expression construct beneficially facilitates transformation of a cell, preferably a plant cell, even more preferably a dicotyledon plant cell as listed herein.

In an expression construct according to the present invention the heterologous polynucleotide preferably comprises a promoter for expressing the Lr67 allele in a plant leaf cell, preferably a soybean plant leaf cell. This way the expression construct can form an expression cassette and particularly facilitates the generation of recombinant or transgenic plants as described herein exhibiting improved resistance against one or more biotrophic or heminecrotrophic fungi as described herein, preferably against Phakopsora pachyrhizi.

As described herein, fungal resistance is most useful where it is observed in a whole plant or plant part. This way, the improved resistance allows for a reduction of fungicide use in commercial farming, thereby reducing farming costs and land exposure to pesticides without compromising yield of the finally harvested material. To this end, the plant or plant part according to the present invention comprises a cell according to the present invention or is transformed with an expression construct according to the present invention. Preferably, the plant of the present invention consists of cells according to the invention. This way, the plant can make use of the Lr67 allele of the present invention in all tissues. However, it is an advantage of the present invention that the plant can also be a chimeric plant, for example a periclinal or plastidal chimera, or can comprise chimeric plant parts, for example chimeric leaves and/or stems. The skilled person understands that even though a chimeric plant offers enhanced resistance against fungal infections, the degree of protection increases according to the content of cells according to the invention forming a plant tissue.

Accordingly, the preferably recombinant or transgenic plant or plant part of the present invention has reduced or abolished susceptibility, relative to a wild type plant, to infections by a biotrophic or heminecrotrophic fungus, preferably a rust fungus, more preferably a fungus of phylum Basidiomycota, even more preferably of subphylum Pucciniomycotina, even more preferably of class Pucciniomycetes, even more preferably of order Pucciniales, even more preferably of family Chaconiaceae, Coleosporiaceae, Cronartiaceae, Melampsoraceae, Mikronegeriaceae, Phakopsoraceae, Phragmidiaceae, Pileolariaceae, Pucciniaceae, Pucciniastraceae, Pucciniosiraceae, Raveneliaceae, Sphaerophragmiaceae or Uropyxidaceae, even more preferably of genus Maravalia, Ochropsora, Olivea, Chrysomyxa, Coleosporium, Diaphanopellis, Cronartium, Endocronartium, Peridermium, Melampsora, Chrysocelis, Mikronegeria, Arthuria, Batistopsora, Cerotelium, Dasturella, Phakopsora, Prospodium, Arthuriomyces, Catenulopsora, Gerwasia, Gymnoconia, Hamaspora, Kuehneola, Phragmidium, Trachyspora, Triphragmium, Atelocauda, Pileolaria, Racospermyces, Uromycladium, Allodus, Ceratocoma, Chrysocyclus, Cumminsiella, Cystopsora, Endophyllum, Gymnosporangium, Miyagia, Puccinia, Puccorchidium, Roestelia, Sphenorchidium, Stereostratum, Uromyces, Hyalopsora, Melampsorella, Melampsoridium, Milesia, Milesina, Naohidemyces, Pucciniastrum, Thekopsora, Uredinopsis, Chardoniella, Dietelia, Pucciniosira, Diorchidium, Endoraecium, Kernkampella, Ravenelia, Sphenospora, Austropuccinia, Nyssopsora, Sphaerophragmium, Dasyspora, Leucotelium, Macruropyxis, Porotenus, Tranzschelia or Uropyxis, most preferably of species Phakopsora pachyrhizi, Phakopsora meibomiae, Puccinia graminis, Puccinia striiformis, Puccinia hordei or Puccinia recondita. Such beneficial resistance is conveyed for at least one of the listed fungal pathogens. It is preferred that such resistance is for a pathogen of genus Phakopsora, even more preferably for a pathogen of species Phakopsora pachyrhizi, and at least one further fungal pathogen, preferably of genus Fusarium, Rhizoctonia and/or Mycospaerella.

The plant or plant part according to the present invention makes use of an Lr67 allele of the present invention to achieve the advantages described herein. Thus, a plant or plant part is preferred
i) wherein the Lr67 allele is integral in the, preferably nuclear, genome of the plant or plant part, and/or
ii) wherein the plant or plant part is homozygous for the Lr67 gene or, less preferred, heterozygous for the Lr67 gene, and/or
iii) wherein the plant or plant part, when in meiosis, is non-segregating or, less preferred, segregating for the Lr67 allele, and/or
iv) wherein the Lr67 allele is operably linked to a heterologous promoter, and/or
v) wherein the Lr67 allele is, in the genome of the plant or plant part, integrated at a different locus than the corresponding wild type Lr67 gene, and/or
vi) wherein the plant or plant part is transgenic or wherein the mutation in the Lr67 gene is an artificially induced heritable allele.

Each of the alternatives is linked to advantages as described herein. It is understood that most preferably the Lr67 allele of the present invention is integral in the genome of the plant or plant part, thereby providing a stable enhancement of resistance. Such plants or plant parts according to the invention can be transgenic, thereby preferably comprising the Lr67 allele operably linked to the heterologous promoter—preferred promoters are described herein —, and/or integrated at a different locus than the corresponding wild type Lr67 gene. As described herein, it is a particular advantage of the present invention that the enhancement of resistance does not depend on the inactivation or removal of the corresponding wild type soybean Lr67 gene. However, in order to reduce the likelihood of unwanted homologous recombination events it is preferred that in the plant, plant part or cell of the present invention at least one, preferably all wild-type Lr67 genes is/are (a) replaced by the Lr67 allele of the present invention or (b) inactivated and complemented by the Lr67 allele of the present invention. Such plants, plant parts or cells can be transgenic or can be the result of an artificially induced heritable genomic alteration as described herein.

Most preferably, the plant is or the part thereof belongs to a monocotyledon or dicotyledon plant, more preferably a plant of order Fabales, more preferably a plant of family Fabaceae, more preferably a plant of tribus Phaseoleae, more preferably of genus Glycine, most preferably of species Glycine max, Glycine soja, Glycine gracilis or a cross Glycine max×Glycine soja. Such plants are naturally highly susceptible to infections by rust fungi, in particular to infections by a fungus of species Phakopsora pachyrhizi, presently leading to an intensive use of fungicides. The invention allows to reduce the number of fungicide treatments per plant growing season.

Thus, the invention provides a method for growing a plant having an Lr67 allele of the present invention on a field, preferably a plant of order Fabales, more preferably a plant of family Fabaceae, more preferably a plant of tribus Phaseoleae, more preferably of genus Glycine, most preferably of species Glycine max, Glycine soja, Glycine gracilis or a cross Glycine max×Glycine soja, comprising the step of treating the plant with a fungizide effective against a biotrophic or heminecrotrophic fungus, preferably a rust fungus, more preferably a fungus of phylum Basidiomycota, even more preferably of subphylum Pucciniomycotina, even more preferably of class Pucciniomycetes, even more preferably of order Pucciniales, even more preferably of family Chaconiaceae, Coleosporiaceae, Cronartiaceae, Melampsoraceae, Mikronegeriaceae, Phakopsoraceae, Phragmidiaceae, Pileolariaceae, Pucciniaceae, Pucciniastraceae, Pucciniosiraceae, Raveneliaceae, Sphaerophragmiaceae or Uropyxidaceae, even more preferably of genus Maravalia, Ochropsora, Olivea, Chrysomyxa, Coleosporium, Diaphanopellis, Cronartium, Endocronartium, Peridermium, Melampsora, Chrysocelis, Mikronegeria, Arthuria, Batistopsora, Cerotelium, Dasturella, Phakopsora, Prospodium, Arthuriomyces, Catenulopsora, Gerwasia, Gymnoconia, Hamaspora, Kuehneola, Phragmidium, Trachyspora, Triphragmium, Atelocauda, Pileolaria, Racospermyces, Uromycladium, Allodus, Ceratocoma, Chrysocyclus, Cumminsiella, Cystopsora, Endophyllum, Gymnosporangium, Miyagia, Puccinia, Puccorchidium, Roestelia, Sphenorchidium, Stereostratum, Uromyces, Hyalopsora, Melampsorella, Melampsoridium, Milesia, Milesina, Naohidemyces, Pucciniastrum, Thekopsora, Uredinopsis, Chardoniella, Dietelia, Pucciniosira, Diorchidium, Endoraecium, Kernkampella, Ravenelia, Sphenospora, Austropuccinia, Nyssopsora, Sphaerophragmium, Dasyspora, Leucotelium, Macruropyxis, Porotenus, Tranzschelia or Uropyxis, even more preferably of species Phakopsora pachyrhizi, Phakopsora meibomiae, Puccinia graminis, Puccinia striiformis, Puccinia hordei or Puccinia recondita and most preferably of species Phakopsora pachyrhizi, wherein the number of fungizide treatments during the growth of the plant is reduced by at least one compared to the treatment of a wild-type plant of the same species growing under the same conditions, even more preferably reduced by two treatments. This is made feasible due to the effect of the Lr67 allele of the present invention in the plant. Preferably, the plant is exposed to at most 3 treatments in a region where, for a wild-type plant, 5 fungicide treatments would be agronomically required, and at most 2 treatments in a region where, for a wild-type plant, 3 fungicide treatments would be agronomically required.

Thus, the invention also provides an ensemble of at least 50 plants according to the present invention, more preferably at least 100 plants, even more preferably at least 1000 plants, even more preferably at least 100000 plants. According to the invention, preferably at least 100000 plants are grown per hectare, more preferably 200000 to 800000 plants per hectare, even more preferably at least 250000 to 650000 plants per hectare. Such plant numbers preferably are observed within one hectare; thus, the invention particularly facilitates ecologically considerate intensive farming with reduced use of fungicides per growing season. The plants according to the invention are preferably growing in a field or greenhouse. The term “ensemble” in particular encompasses any collection of plants linked to one another by proximity, such as plants on a tray or on a field.

According to the invention it is not required that all plants of one species growing in the same field or greenhouse are plants of the present invention. Instead, it is sufficient in monoculture plantation if at least about 25% of the plants of one species belong to the present invention, more preferably at least 50%, even more preferably 25%-75% and most preferably 45%-70%, especially when mixed or combined with plants harboring other resistance genes or mechanisms. The combination with plants with other resistance gene can be done by interplanting (mixing), row-wise or blockwise. For example, on a soybean field it is possible to reduce the number of fungicide treatments if approximately every second plant is a plant according to the present invention. It is particularly preferred that at least 25%, more preferably 50%-100% and even more preferably 75%-100% of those plants on the same field that are not plants according to the present invention comprise at least one other biological means for enhancing fungal resistance, most preferably the other means is selected from the list of pathogen resistance polypeptides as described above.

According to the invention there is provided a method for control of a fungal pathogen, comprising the steps of
a) planting a plant according to the present invention, and
b) applying, to at least one of (a) the plant, (b) an area adjacent to the plant, (c) soil adapted to support growth of the plant, (d) a root of the plant, and (e) foliage of the plant, a fungicidally effective amount of a composition comprising one or more fungicides effective against one or more necrotrophic fungi.

As described herein it is a particular advantage of the Lr67 alleles of the present invention to impart or increase resistance against a biotrophic and/or a heminecrotrophic fungus. Thus, for the control and prevention of fungal attack on or infection of a plant, it is not necessary to use as much of fungicides effective against such fungus or fungi. Instead the farmer can focus on defending against necrotrophic fungi.

According to the invention there are also provided a seed, flower, leaf, fruit, processed food, or food ingredient from the plant according to the invention, preferably an accumulation of at least 1 kg of such seeds, even more preferably at least 1000 kg of such seeds. For the purposes of the present invention, the seeds are stored and weighted without pods. The term “accumulation” in particular comprises any spatial arrangement of seeds, non-limiting examples are seeds in close vicinity without complete enclosure, such as seeds on a pile or heap, or seeds in close vicinity enclosed by an envelope, such as seeds in a bag, package or barrel.

The invention also provides a use of a plant according to the invention or of an ensemble according to the invention, or of a part of such plant or ensemble, as animal feed or to produce a feed product for animal or a food product for human consumption, preferably (a) wherein the food product is a wholemeal, meal or starch, or (b) wherein the food product is a fermentation product of the part of the plant or ensemble, preferably a fermentation product of seed, or (c) wherein the feed or food product is an oil.

Preferred according to the invention is a method for creating a cell as described herein, comprising the step of transforming a cell with a nucleic acid coding for an Lr67 allele as described herein. This way a transgenic or recombinant plant can be produced using standard techniques known to the skilled person without unusual burden.

Further preferred according to the invention is a method for creating a cell according to the invention, comprising the steps of
i) introducing into a cell an enzyme having endonuclease or nickase activity and recognizing a soybean Lr67 gene sequence,
ii) effecting by action of said enzyme a change in nucleotide sequence such as to introduce an amino acid change at position 145 and optionally also at position 389, using the numbering according to SEQ ID NO. 1.

This way, a non-transgenic plant comprising the Lr67 allele of the present invention can be produced. As described herein, the Lr67 allele of the present invention differs from a soybean Lr67 gene in at least the one, preferably at least the two amino acid substitutions as described herein. The skilled person understands that, particularly where the method for creating a cell according to the invention involves the use of undirected mutagenesis tools such that the substitution effected cannot be predicted, it is advantageous to verify that the amino acid change or changes at position 145 and preferably also at position 389 are substitutions as described above according to the invention. As the phenotype achievable by the Lr67 allele of the present invention is easy to observe, such verification can be done by growing a plant and verifying that it shows enhanced resistance against the biotrophic or heminecrotrophic fungus, preferably against a rust fungus.

In such method, preferably the enzyme is functionally linked to a mutation inducing agent, preferably a deaminase, glycosylase or a chemical agent, preferably acridine, psoralen, and/or ethidium bromide.

A method according to the invention preferably further comprises the steps of growing a plant from said cell and selecting, in comparison to a wild type plant, plants having reduced or abolished susceptibility to infections by a biotrophic or heminecrotrophic fungus, preferably a rust fungus, more preferably a fungus of phylum Basidiomycota, even more preferably of subphylum Pucciniomycotina, even more preferably of class Pucciniomycetes, even more preferably of order Pucciniales, even more preferably of family Chaconiaceae, Coleosporiaceae, Cronartiaceae, Melampsoraceae, Mikronegeriaceae, Phakopsoraceae, Phragmidiaceae, Pileolariaceae, Pucciniaceae, Pucciniastraceae, Pucciniosiraceae, Raveneliaceae, Sphaerophragmiaceae or Uropyxidaceae, even more preferably of genus Maravalia, Ochropsora, Olivea, Chrysomyxa, Coleosporium, Diaphanopellis, Cronartium, Endocronartium, Peridermium, Melampsora, Chrysocelis, Mikronegeria, Arthuria, Batistopsora, Cerotelium, Dasturella, Phakopsora, Prospodium, Arthuriomyces, Catenulopsora, Gerwasia, Gymnoconia, Hamaspora, Kuehneola, Phragmidium, Trachyspora, Triphragmium, Atelocauda, Pileolaria, Racospermyces, Uromycladium, Allodus, Ceratocoma, Chrysocyclus, Cumminsiella, Cystopsora, Endophyllum, Gymnosporangium, Miyagia, Puccinia, Puccorchidium, Roestelia, Sphenorchidium, Stereostratum, Uromyces, Hyalopsora, Melampsorella, Melampsoridium, Milesia, Milesina, Naohidemyces, Pucciniastrum, Thekopsora, Uredinopsis, Chardoniella, Dietelia, Pucciniosira, Diorchidium, Endoraecium, Kernkampella, Ravenelia, Sphenospora, Austropuccinia, Nyssopsora, Sphaerophragmium, Dasyspora, Leucotelium, Macruropyxis, Porotenus, Tranzschelia or Uropyxis, most preferably of species Phakopsora pachyrhizi, Phakopsora meibomiae, Puccinia graminis, Puccinia striiformis, Puccinia hordei or Puccinia recondita.

Preferred according to the invention is a method for producing a population of plants each having an enhanced resistance to at least one biotrophic or heminecrotrophic fungus, preferably a rust fungus, comprising the steps of
i) providing a plant according to the invention, and
ii) crossing the plant of step a) with a compatible plant without active Lr67 allele,
iii) growing progeny obtained by the crossing of step b).

This way a population, for example for obtaining seed to grow in the next season or for further crossing, can easily be obtained using standard breeding techniques. As described herein it is a particular advantage of the Lr67 allele of the present invention that a single copy of the allele is, by any practical definition, sufficient to induce the advantageous phenotype of increased resistance against the fungus in question, preferably a fungus of genus Phakopsora. This allows for easy transfer of the phenotypic trait also in polyploid plant species.

Preferably the plant in step a) is homozygous for the Lr67 allele. This is of particular advantage because offspring of the plant are according to the Mendelian laws practically guaranteed to share the enhanced resistance phenotype of the homozygous parent plant.

The invention also provides a method for reducing or abolishing susceptibility of a plant or plant part, in comparison to a wild type plant, to infections by a biotrophic or heminecrotrophic fungus, preferably a rust fungus, comprising causing or increasing expression or activity of an Lr67 allele according to the present invention, i.e. an Lr67 allele comprising one or more mutations in a soybean Lr67 gene, wherein the one or more mutations comprise, in the numbering according to SEQ ID NO. 1, a substitution at position G145, preferably a substitution selected from, in decreasing order of preference, G145R, G145K, G145H, G145Q, G145E, G145V, G145L and G145Y, and optionally also a substitution at position 1389, preferably a substitution selected from, in decreasing order of preference, 1389L, 1389W, 1389K, 1389R, 1389Q, 1389F and 1389M.

Preferably the fungus is a fungus of phylum Basidiomycota, even more preferably of subphylum Pucciniomycotina, even more preferably of class Pucciniomycetes, even more preferably of order Pucciniales, even more preferably of family Chaconiaceae, Coleosporiaceae, Cronartiaceae, Melampsoraceae, Mikronegeriaceae, Phakopsoraceae, Phragmidiaceae, Pileolariaceae, Pucciniaceae, Pucciniastraceae, Pucciniosiraceae, Raveneliaceae, Sphaerophragmiaceae or Uropyxidaceae, even more preferably of genus Maravalia, Ochropsora, Olivea, Chrysomyxa, Coleosporium, Diaphanopellis, Cronartium, Endocronartium, Peridermium, Melampsora, Chrysocelis, Mikronegeria, Arthuria, Batistopsora, Cerotelium, Dasturella, Phakopsora, Prospodium, Arthuriomyces, Catenulopsora, Gerwasia, Gymnoconia, Hamaspora, Kuehneola, Phragmidium, Trachyspora, Triphragmium, Atelocauda, Pileolaria, Racospermyces, Uromycladium, Allodus, Ceratocoma, Chrysocyclus, Cumminsiella, Cystopsora, Endophyllum, Gymnosporangium, Miyagia, Puccinia, Puccorchidium, Roestelia, Sphenorchidium, Stereostratum, Uromyces, Hyalopsora, Melampsorella, Melampsoridium, Milesia, Milesina, Naohidemyces, Pucciniastrum, Thekopsora, Uredinopsis, Chardoniella, Dietelia, Pucciniosira, Diorchidium, Endoraecium, Kernkampella, Ravenelia, Sphenospora, Austropuccinia, Nyssopsora, Sphaerophragmium, Dasyspora, Leucotelium, Macruropyxis, Porotenus, Tranzschelia or Uropyxis, most preferably of species Phakopsora pachyrhizi, Puccinia graminis, Puccinia striiformis, Puccinia hordei or Puccinia recondita.

Further preferably the Lr67 gene
a) codes for a polypeptide having an amino acid sequence of at least 40%, more preferably at least 50%, more preferably at least 60%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%-84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98% or 99% identity to any of SEQ ID NO. 1, 2, 3, 4, 5, 6 or 7,
or a biologically active fragment thereof, and/or
b) comprises a nucleic acid sequence
   obtainable or obtained by amplification from the genome of Glycine max using any of the primer pairs according to SEQ ID NO. 15 and 21, 16 and 22, 17 and 23, 18 and 24, 19 and 25, or 20 and 26, and/or
   obtainable or obtained by reverse translation from an RNA hybridizing under stringent conditions to a nucleic acid probe according to SEQ ID NO. 27, 28, 29, 30, 31 or 32, and/or
   having at least 60%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%-84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98% or 99% identity to any of SEQ ID NO. 33, 34, 35, 36, 37 or 38, and/or
c) comprises a PF00083 Pfam domain, an InterPro domain selected from IPR020846, IPR005828, IPR036259, IPR003663 and IPR005829, a Prosite PS50850 Major facilitator superfamily (MFS) profile and/or 12 transmembrane domains.

According to the present invention, the term “biologically active” signifies that the such attributed fragment has essentially the same functionality as the full polypeptide or nucleic acid sequence. For the purposes of the present invention, a fragment is biologically active if it is sufficient to induce, impart, stabilize or increase resistance of a plant cell, preferably of order Fabales, more preferably a plant of family Fabaceae, more preferably a plant of tribus Phaseoleae, more preferably of genus Glycine, most preferably of species Glycine max, Glycine soja, Glycine gracilis or a cross Glycine max×Glycine soja, against a fungus of species Phakopsora pachyrhizi.

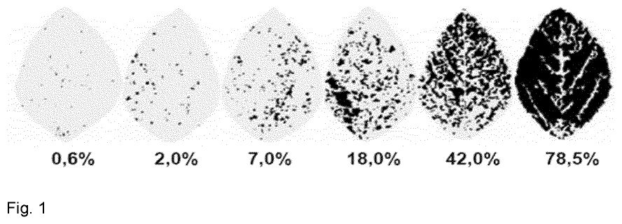

The effect of the Lr67 allele according to the invention preferably is measured by assessment of diseased leaf area of mature plants as described with respect to FIG. 1 herein. The Lr67 allele of the present invention leads to a reduction of diseased leaf area in mature plants compared to a corresponding wild type plant. Preferably, the Lr67 allele of the present invention leads to a reduction of diseased leaf area, compared to that of a wild type plant set to 100%, in non-fungicidally treated plants to less than 60%, more preferably less than 50%, more preferably less than 40%. Thus, where a wild type plant exhibits 60% diseased leaf area, a plant according to the present invention, i.e. one in which the Lr67 allele of the present invention is active, preferably exhibits a diseased leaf area of preferably less than approximately 40%, preferably less than approximately 30% and most preferably at most 25%.

Further preferably the Lr67 gene in such method
a) codes for a polypeptide having an amino acid sequence of at least 40%, more preferably at least 50%, more preferably at least 60%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%-84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98% or 99% identity to any of SEQ ID NO. 1, 2, 3, 4, 5, 6 or 7,
or a biologically active fragment thereof, and/or
b) comprises a nucleic acid sequence
   obtainable or obtained by amplification from the genome of Glycine max using any of the primer pairs according to SEQ ID NO. 15 and 21, 16 and 22, 17 and 23, 18 and 24, 19 and 25, or 20 and 26, and/or
   obtainable or obtained by reverse translation from an RNA hybridizing under stringent conditions to a nucleic acid probe according to SEQ ID NO. 27, 28, 29, 30, 31 or 32, and/or
   having at least 60%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%-84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98% or 99% identity to any of SEQ ID NO. 33, 34, 35, 36, 37 or 38, and/or
c) comprises a PF00083 Pfam domain, an InterPro domain selected from IPR020846, IPR005828, IPR036259, IPR003663 and IPR005829, a Prosite PS50850 Major facilitator superfamily (MFS) profile and/or 12 transmembrane domains.

And also preferably the plant or plant part in such method is transgenic plant or the mutation in the Lr67 gene is an artificially induced heritable allele. The latter can be introduced into the plant genome for example using an enzyme having endonuclease or nickase activity and recognizing a soybean Lr67 gene sequence and effecting by action of said enzyme a change in nucleotide sequence such as to introduce an amino acid change at position 145, using the numbering according to SEQ ID NO. 1.

Preferred is such method
i) wherein the Lr67 allele is integral in the, preferably nuclear, genome of the plant or plant part, and/or
ii) wherein the plant or plant part is homozygous for the Lr67 gene or, less preferred, heterozygous for the Lr67 gene, and/or
iii) wherein the plant or plant part, when in meiosis, is non-segregating or, less preferred, segregating for the Lr67 allele, and/or
iv) wherein the Lr67 allele is operably linked to a heterologous polynucleotide, preferably a heterologous promoter, and/or preferably wherein the heterologous polynucleotide comprises a promoter for expressing the Lr67 allele in a plant leaf cell, preferably a soybean plant leaf cell.
v) wherein the Lr67 allele is, in the genome of the plant or plant part, integrated at a different locus than the corresponding wild type Lr67 gene, and/or
vi) wherein the plant or plant part is transgenic or wherein the mutation in the Lr67 gene is an artificially induced heritable allele, and/or
vii) wherein the plant is or the part thereof belongs to a monocotyledon or dicotyledon plant, more preferably a plant of order fabales, more preferably a plant of family Fabaceae, more preferably a plant of tribus Phaseoleae, more preferably of genus Glycine, most preferably of species Glycine max, Glycine soja, Glycine gracilis or a cross Glycine max ×Glycine soja.

The invention also provides a method of assaying a plant for resistance to a biotrophic or heminecrotrophic fungus, preferably a rust fungus, comprising the screening for the presence of the Lr67 allele in a cell of said plant,
preferably comprising the detection of a polynucleotide, preferably an mRNA,
a) hybridizing under stringent conditions to a nucleic acid probe according to SEQ ID NO. 27, 28, 29, 30, 31 or 32, and/or
b) having at least 60%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%-84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98% or 99% identity to any of SEQ ID NO. 33, 34, 35, 36, 37 or 38.

According to the invention the aforementioned method of assaying a plant for resistance comprises the detection of the presence of a nucleic acid, preferably an mRNA, in one or more cells of the plant. Preferably the detection is performed using any of the nucleic acid probes mentioned above. However, the method is not limited to those probes and instead comprises any nucleic acid detection method for detecting a nucleic acid sequence, as long as the detected polynucleotide hybridizes, under stringent conditions, to at least one of the aforementioned probes. For example, the presence of nucleic acids according to the present invention can also be achieved by amplifying a nucleic acid as described below, preferably verifying that the amplified nucleic acid has the desired length, optionally followed by sequencing of the amplified nucleic acid to verify that a polypeptide coded by the nucleic acid comprises the sequence characteristics, preferably at least comprises an amino acid substitution at position 145 and optionally 389 as described herein.

The soybean Lr67 gene can according to the present invention be obtained by amplification from soybean genetic material using the following primer pairs and probes:

| | | antisense | | | |
|---|---|---|---|---|---|
| gene | sense primer | primer | product | optimal annealing | probe |
| [SEQ ID NO.] | [SEQ ID NO.] | [SEQ ID NO.] | length [nt] | temperature [° C.] | [SEQ ID NO.] |
| 2 | 15 | 21 | 1569 | 55.3 | 27 |
| 3 | 16 | 22 | 1569 | 55.3 | 28 |
| 4 | 17 | 23 | 1422 | 55.3 | 29 |
| 5 | 18 | 24 | 1428 | 54.1 | 30 |
| 6 | 19 | 25 | 1539 | 53.3 | 31 |
| 7 | 20 | 26 | 1503 | 50.4 | 32 |

The assaying method of the present invention is particularly useful in assisting the propagation of plants, preferably crops, in that it allows to screen seed fungal resistance percentage before shipment and/or planting. The skilled person understands that due to the sequence similarity of the amplified products more than one probe can be capable of hybridizing thereto under stringent conditions.

The invention therefore also provides a method of propagation of a sexually reproducing plant, comprising:

i) obtaining a plurality of seeds of a plant, preferably a monocotyledon or dicotyledon plant, more preferably a plant of order Fabales, more preferably a plant of family Fabaceae, more preferably a plant of tribus Phaseoleae, more preferably of genus Glycine, most preferably of species Glycine max, Glycine soja, Glycine gracilis or a cross Glycine max×Glycine soja,

ii) ascertaining that at least about 25%, more preferably at least 50%, even more preferably 25%-95% and most preferably 45%-100% of the seed of one species comprise an Lr67 allele according to the present invention, and

iii) planting seed of the plurality of seeds satisfying the condition according to step ii).

The invention thus advantageously allows to grow and harvest seeds of a plant of the present invention in an essentially soybean rust free environment, for example in a climate zone where soybean rust normally cannot survive in non-growth season, for example in the United States of America: Arkansas, Illinois, Indiana, Iowa, Kansas, Kentucky, Louisiana, Michigan, Minnesota, Mississippi, Missouri, Nebraska, North Carolina, North Dakota, Ohio, South Dakota, Tennessee and/or Wisconsin, and, after ascertaining that the content of seed according to the present invention is sufficiently high, planting all or some of the seeds in an environment susceptible to soybean rust infection, for example in Brazil.

The invention is hereinafter further described by way of examples and selected preferred embodiments. Neither the examples nor the selected embodiments are intended to limit the scope of the claims.

### EXAMPLES

### Example 1: General Methods

The chemical synthesis of oligonucleotides can be affected, for example, in the known fashion using the phosphoamidite method (Voet, Voet, 2nd Edition, Wiley Press New York, pages 896-897). The cloning steps carried out for the purposes of the present invention such as, for example, restriction cleavages, agarose gel electrophoresis, purification of DNA fragments, transfer of nucleic acids to nitrocellulose and nylon membranes, linking DNA fragments, transformation of E. coli cells, bacterial cultures, phage multiplication and sequence analysis of recombinant DNA, are carried out as described by Sambrook et al. Cold Spring Harbor Laboratory Press (1989), ISBN 0-87969-309-6. The sequencing of recombinant DNA molecules is carried out with an MWG-Licor laser fluorescence DNA sequencer following the method of Sanger (Sanger et al., Proc. Natl. Acad. Sci. USA 74, 5463 (1977).

### Example 2: Cloning of Glyma.01g238800.1G145R_V389L and TaLr67res

The cDNA sequence of Glyma.01g238800.1G145R_V389L and TaLr67res genes mentioned in this application were generated by DNA synthesis (Geneart, Regensburg, Germany).

The Glyma.01g238800.1G145R_V389L (as shown in SEQ ID NO. 9) was synthesized in a way that a Ascl restriction site is located in front of the start-ATG and a Sbfl restriction site downstream of the stop-codon. The synthesized DNA was digested using the restriction enzymes Sbfl and Ascl (NEB Biolabs) and ligated in a Sbfl/Ascl digested Gateway pENTRY-B vector (Invitrogen, Life Technologies, Carlsbad, Calif., USA) in a way that the full-length fragment is located in sense direction between the parsley ubiquitin promoter and the Agrobacterium tumefaciens derived nopaline synthase terminator (t-nos). The PcUbi promoter regulates constitutive expression of the ubi4-2 gene (accession number X64345) of Petroselinum crispum (Kawalleck et al. 1993 Plant Molecular Biology 21(4): 673-684).

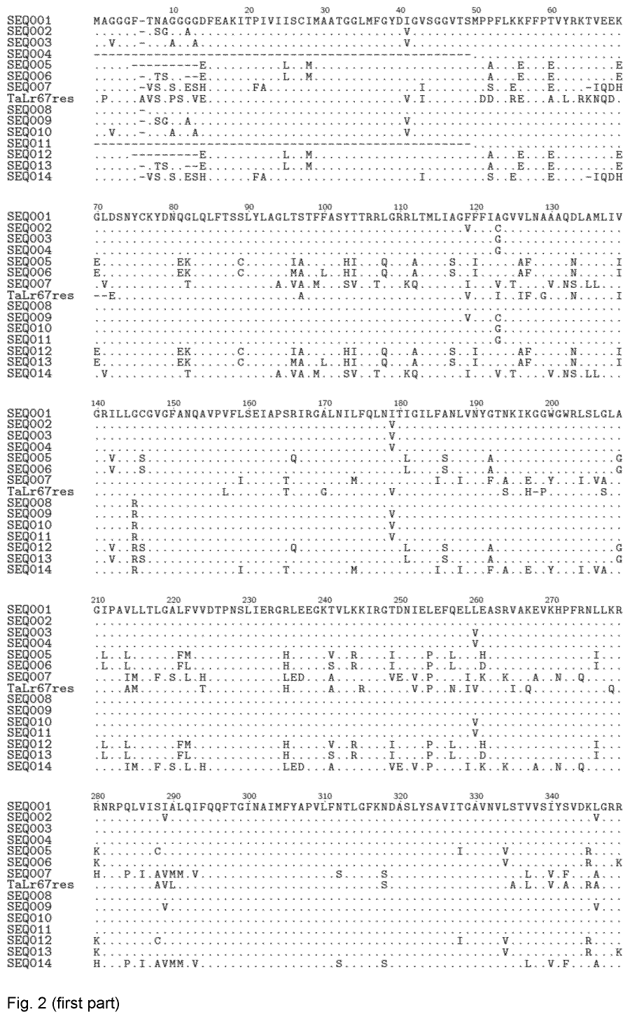

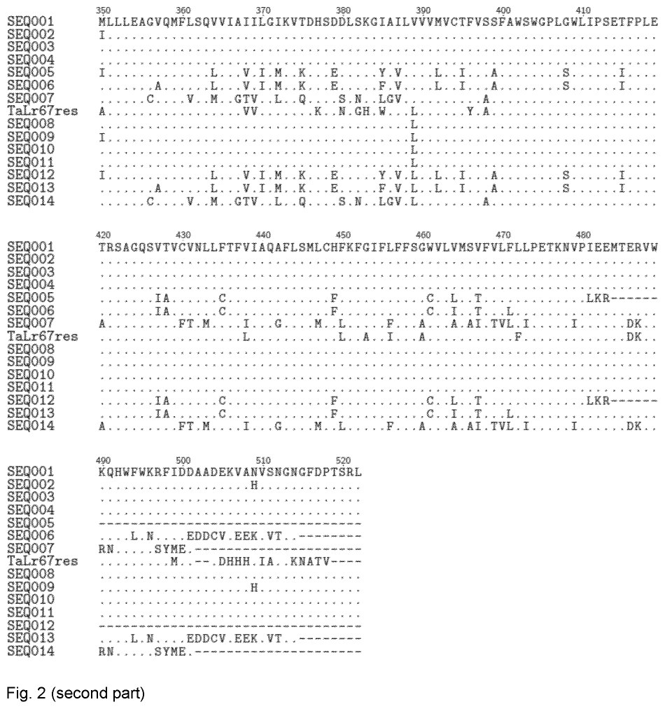

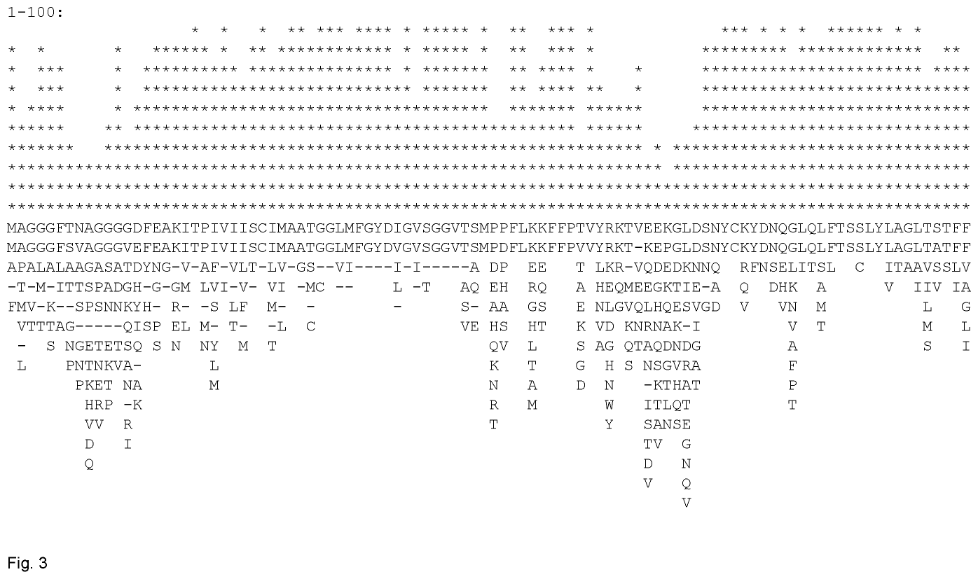

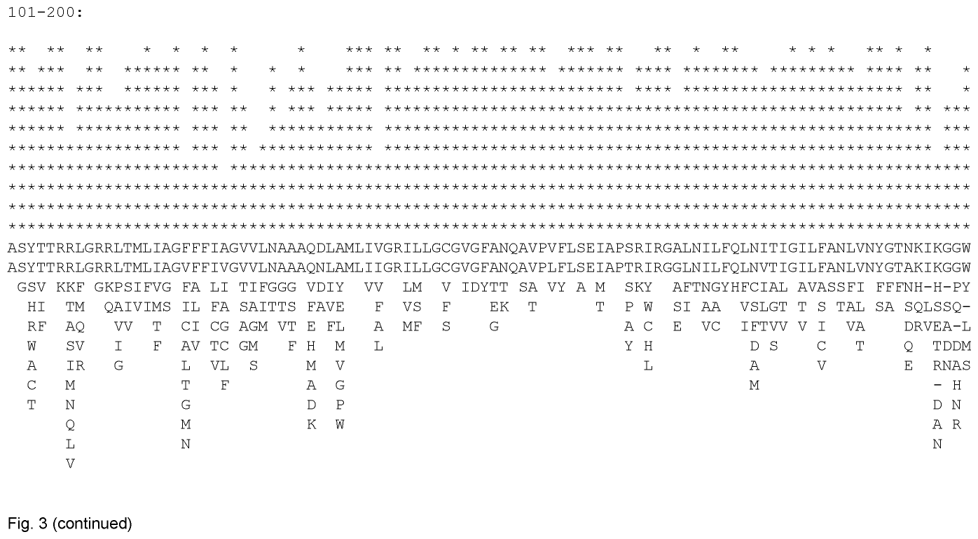

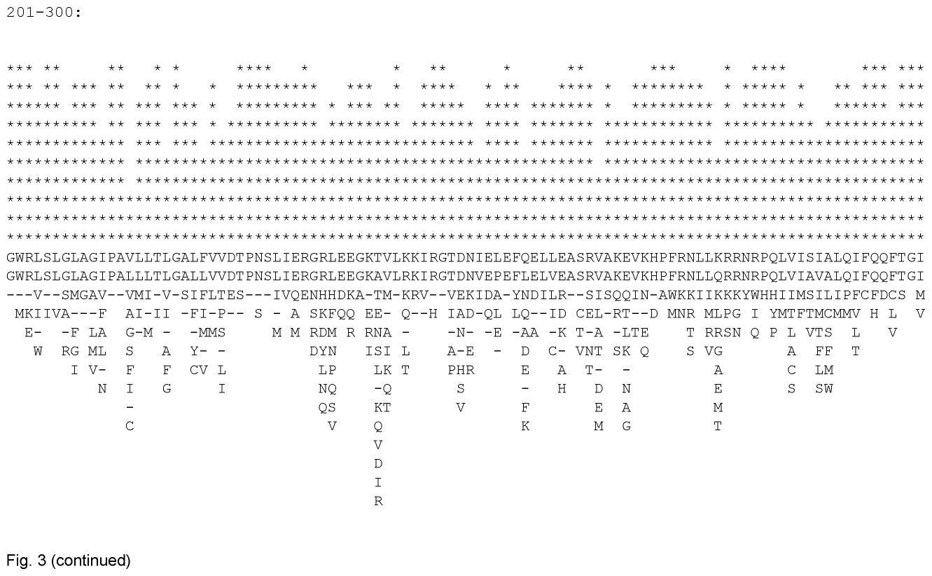

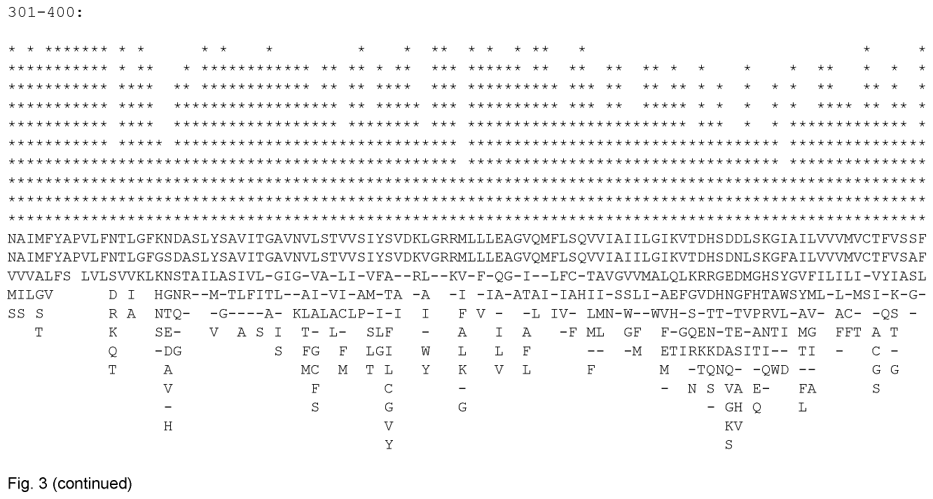

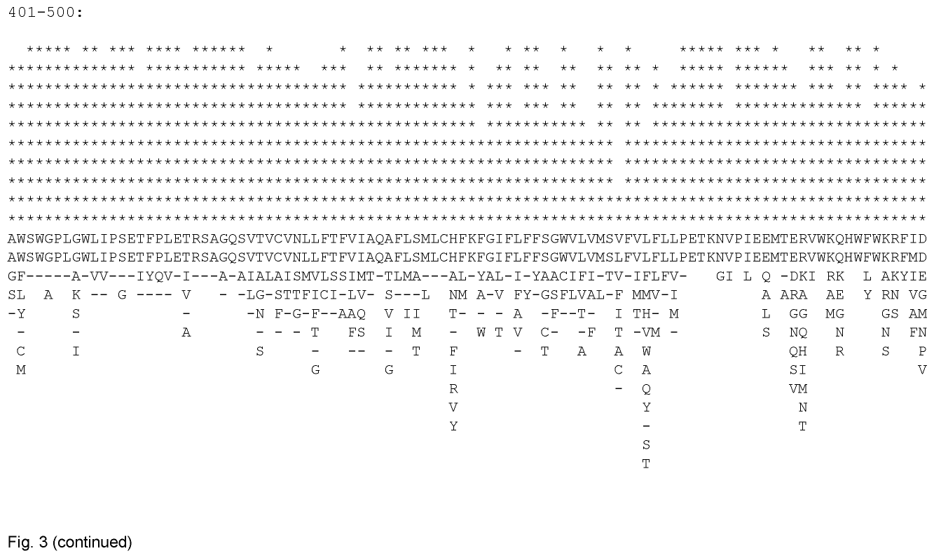

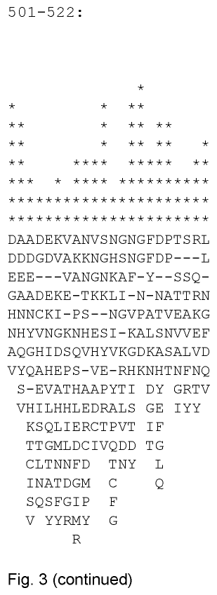

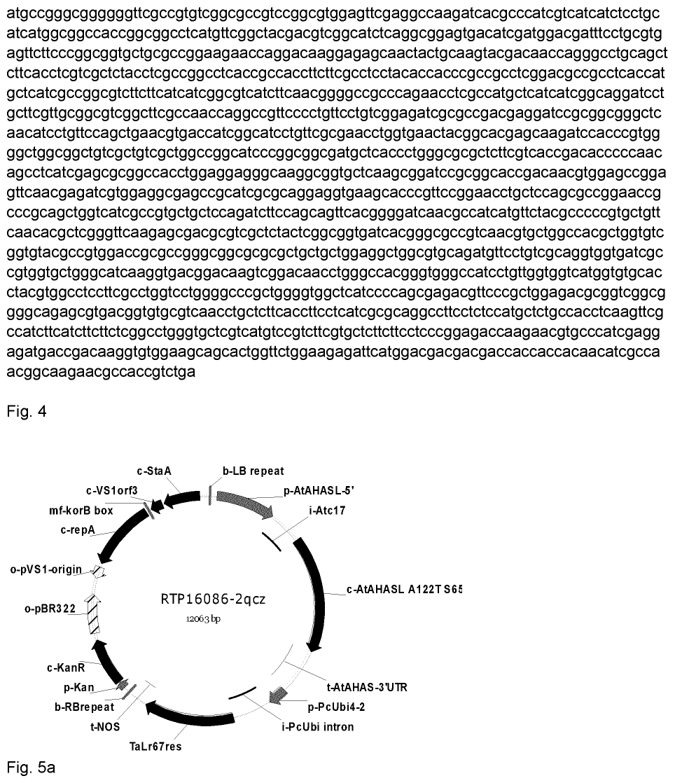

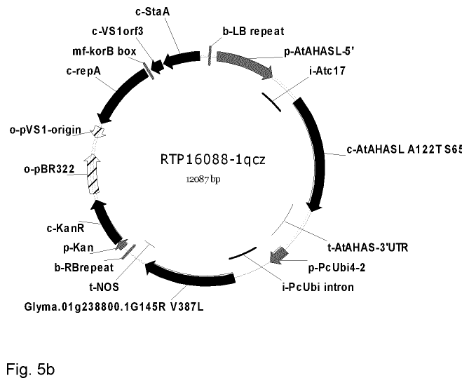

To obtain the binary plant transformation vector, a triple LR reaction (Gateway system, Invitrogen, Life Technologies, Carlsbad, Calif., USA) was performed according to manufacturer's protocol by using an empty pENTRY-A vector, the PcUbi promoter:: Glyma.01g238800.1G145R_V389L:nos-terminator in the above described pENTRY-B vector and an empty pENTRY-C. As target a binary pDEST vector was used which is composed of: (1) a Kanamycin resistance cassette for bacterial selection (2) a pVS1 origin for replication in Agrobacteria (3) a ColE1 origin of replication for stable maintenance in E. coli and (4) between the right and left border an AHAS selection under control of a PcUbi-promoter (see FIG. 2). The recombination reaction was transformed into E. coli (DH5alpha), mini-prepped and screened by specific restriction digestions. A positive clone from each vector construct was sequenced and submitted soy transformation.

The TaLr67res gene (as shown in FIG. 2) was synthesized in a way that a Ascl restriction site is located in front of the start-ATG and a Sbfl restriction site downstream of the stop-codon. The synthesized DNA was digested using the restriction enzymes Sbfl and Ascl (NEB Biolabs) and ligated in a Sbfl/Ascl digested Gateway pENTRY-B vector (Invitrogen, Life Technologies, Carlsbad, Calif., USA) in a way that the full-length fragment is located in sense direction between the parsley ubiquitin promoter and the Agrobacterium tumefaciens derived nopaline synthase terminator (t-nos). The PcUbi promoter regulates constitutive expression of the ubi4-2 gene (accession number X64345) of Petroselinum crispum (Kawalleck et al. 1993 Plant Molecular Biology 21(4): 673-684).

To obtain the binary plant transformation vector, a triple LR reaction (Gateway system, Invitrogen, Life Technologies, Carlsbad, Calif., USA) was performed according to manufacturer's protocol by using an empty pENTRY-A vector, the PcUbi promoter:: TaLr67res::nos-terminator in the above described pENTRY-B vector and an empty pENTRY-C. As target a binary pDEST vector was used which is composed of: (1) a Kanamycin resistance cassette for bacterial selection (2) a pVS1 origin for replication in Agrobacteria (3) a ColE1 origin of replication for stable maintenance in E. coli and (4) between the right and left border an AHAS selection under control of a PcUbi-promoter (see FIG. 2). The recombination reaction was transformed into E. coli (DH5alpha), mini-prepped and screened by specific restriction digestions. A positive clone from each vector construct was sequenced and submitted soy transformation.

### Example 3: Soy Transformation

The expression vector constructs (see example 2) is transformed into soy.

3.1 Sterilization and Germination of Soy Seeds

Virtually any seed of any soy variety can be employed in the method of the invention. A variety of soybean cultivar (including Jack, Williams 82, Jake, Stoddard, CD215 and Resnik) is appropriate for soy transformation. Soy seeds are sterilized in a chamber with a chlorine gas produced by adding 3.5 ml 12N HCl drop wise into 100 ml bleach (5.25% sodium hypochlorite) in a desiccator with a tightly fitting lid. After 24 to 48 hours in the chamber, seeds are removed and approximately 18 to 20 seeds are plated on solid GM medium with or without 5 μM 6-benzyl-aminopurine (BAP) in 100 mm Petri dishes. Seedlings without BAP are more elongated and roots develop especially secondary and lateral root formation. BAP strengthens the seedling by forming a shorter and stockier seedling.

Seven-day-old seedlings grown in the light (>100 pEinstein/m2s) at 25° C. are used for explant material for the three-explant types. At this time, the seed coat was split, and the epicotyl with the unifoliate leaves are grown to, at minimum, the length of the cotyledons. The epicotyl should be at least 0.5 cm to avoid the cotyledonary-node tissue (since soycultivars and seed lots may vary in the developmental time a description of the germination stage is more accurate than a specific germination time).

For inoculation of entire seedlings, see Method A (example 8.3. and 8.3.2) or leaf explants see Method B (example 8.3.3).

For method C (see example 8.3.4), the hypocotyl and one and a half or part of both cotyledons are removed from each seedling. The seedlings are then placed on propagation media for 2 to 4 weeks. The seedlings produce several branched shoots to obtain explants from. The majority of the explants originated from the plantlet growing from the apical bud. These explants are preferably used as target tissue.

3.2—Growth and Preparation of Agrobacterium Culture

Agrobacterium cultures are prepared by streaking Agrobacterium (e.g., A. tumefaciens or A. rhizogenes) carrying the desired binary vector (e.g. H. Klee. R. Horsch and S. Rogers 1987 Agrobacterium-Mediated Plant Transformation and its further Applications to Plant Biology; Annual Review of Plant Physiology Vol. 38: 467-486) onto solid YEP growth medium YEP media: 10 g yeast extract. 10 g Bacto Peptone. 5 g NaCl. Adjust pH to 7.0, and bring final volume to 1 liter with H2O, for YEP agar plates add 20 g Agar, autoclave) and incubating at 25° C. until colonies appeared (about 2 days). Depending on the selectable marker genes present on the Ti or Ri plasmid, the binary vector, and the bacterial chromosomes, different selection compounds are to be used for A. tumefaciens and A. rhizogenes selection in the YEP solid and liquid media. Various Agrobacterium strains can be used for the transformation method.

After approximately two days, a single colony (with a sterile toothpick) is picked and 50 ml of liquid YEP is inoculated with antibiotics and shaken at 175 rpm (25° C.) until an OD600 between 0.8-1.0 is reached (approximately 2 d). Working glycerol stocks (15%) for transformation are prepared and one-ml of Agrobacterium stock aliquoted into 1.5 ml Eppendorf tubes then stored at −80° C.

The day before explant inoculation, 200 ml of YEP are inoculated with 5 μl to 3 ml of working Agrobacterium stock in a 500 ml Erlenmeyer flask. The flask is shaken overnight at 25° C. until the OD600 is between 0.8 and 1.0. Before preparing the soy explants, the Agrobacteria ARE pelleted by centrifugation for 10 min at 5,500xg at 20° C. The pellet Is resuspended in liquid CCM to the desired density (0D600 0.5-0.8) and placed at room temperature at least 30 min before use.

3.3—Explant Preparation and Co-Cultivation (Inoculation)

3.3.1 Method A: Explant Preparation on the Day of Transformation.

Seedlings at this time had elongated epicotyls from at least 0.5 cm but generally between 0.5 and 2 cm. Elongated epicotyls up to 4 cm in length are successfully employed. Explants are then prepared with: i) with or without some roots, ii) with a partial, one or both cotyledons, all preformed leaves are removed including apical meristem, and the node located at the first set of leaves is injured with several cuts using a sharp scalpel.

This cutting at the node not only induces Agrobacterium infection but also distributes the axillary meristem cells and damaged pre-formed shoots. After wounding and preparation, the explants are set aside in a Petri dish and subsequently co-cultivated with the liquid CCM/Agrobacterium mixture for 30 minutes. The explants are then removed from the liquid medium and plated on top of a sterile filter paper on 15×100 mm Petri plates with solid co-cultivation medium. The wounded target tissues are placed such that they are in direct contact with the medium.

3.3.2 Modified Method A: Epicotyl Explant Preparation

Soyepicotyl segments prepared from 4 to 8 d old seedlings are used as explants for regeneration and transformation. Seeds of soya cv. L00106CN, 93-41131 and Jack are germinated in 1/10 MS salts or a similar composition medium with or without cytokinins for 4 to 8 d. Epicotyl explants are prepared by removing the cotyledonary node and stem node from the stem section. The epicotyl is cut into 2 to 5 segments. Especially preferred are segments attached to the primary or higher node comprising axillary meristematic tissue.

The explants are used for Agrobacterium infection. Agrobacterium AGL1 harboring a plasmid with the gene of interest (G01) and the AHAS, bar or dsdA selectable marker gene is cultured in LB medium with appropriate antibiotics overnight, harvested and resuspended in a inoculation medium with acetosyringone. Freshly prepared epicotyl segments are soaked in the Agrobacterium suspension for 30 to 60 min and then the explants were blotted dry on sterile filter papers. The inoculated explants are then cultured on a co-culture medium with L-cysteine and TTD and other chemicals such as acetosyringone for increasing T-DNA delivery for 2 to 4 d. The infected epicotyl explants are then placed on a shoot induction medium with selection agents such as imazapyr (for AHAS gene), glufosinate (for bar gene), or D-serine (for dsdA gene). The regenerated shoots are subcultured on elongation medium with the selective agent.

For regeneration of transgenic plants the segments are then cultured on a medium with cytokinins such as BAP, TDZ and/or Kinetin for shoot induction. After 4 to 8 weeks, the cultured tissues are transferred to a medium with lower concentration of cytokinin for shoot elongation. Elongated shoots are transferred to a medium with auxin for rooting and plant development. Multiple shoots are regenerated.

Many stable transformed sectors showing strong cDNA expression are recovered. Soybean plants are regenerated from epicotyl explants. Efficient T-DNA delivery and stable transformed sectors are demonstrated.

3.3.3 Method B: Leaf Explants

For the preparation of the leaf explant the cotyledon is removed from the hypocotyl. The cotyledons are separated from one another and the epicotyl is removed. The primary leaves, which consist of the lamina, the petiole, and the stipules, are removed from the epicotyl by carefully cutting at the base of the stipules such that the axillary meristems are included on the explant. To wound the explant as well as to stimulate de novo shoot formation, any pre-formed shoots are removed and the area between the stipules was cut with a sharp scalpel 3 to 5 times.

The explants are either completely immersed or the wounded petiole end dipped into the Agrobacterium suspension immediately after explant preparation. After inoculation, the explants are blotted onto sterile filter paper to remove excess Agrobacterium culture and place explants with the wounded side in contact with a round 7 cm Whatman paper overlaying the solid CCM medium (see above). This filter paper prevents A. tumefaciens overgrowth on the soy-explants. Wrap five plates with Parafilm.™. “M” (American National Can, Chicago, Ill., USA) and incubate for three to five days in the dark or light at 25° C.

3.3.4 Method C: Propagated Axillary Meristem

For the preparation of the propagated axillary meristem explant propagated 3-4 week-old plantlets are used. Axillary meristem explants can be pre-pared from the first to the fourth node. An average of three to four explants could be obtained from each seedling. The explants are prepared from plantlets by cutting 0.5 to 1.0 cm below the axillary node on the internode and removing the petiole and leaf from the explant. The tip where the axillary meristems lie is cut with a scalpel to induce de novo shoot growth and allow access of target cells to the Agrobacterium. Therefore, a 0.5 cm explant included the stem and a bud.

Once cut, the explants are immediately placed in the Agrobacterium suspension for 20 to 30 minutes. After inoculation, the explants are blotted onto sterile filter paper to remove excess Agrobacterium culture then placed almost completely immersed in solid CCM or on top of a round 7 cm filter paper overlaying the solid CCM, depending on the Agrobacterium strain. This filter paper prevents Agrobacterium overgrowth on the soy-explants. Plates are wrapped with Parafilm.™. “M” (American National Can, Chicago, Ill., USA) and incubated for two to three days in the dark at 25° C.

3.4—Shoot Induction

After 3 to 5 days co-cultivation in the dark at 25° C., the explants are rinsed in liquid SIM medium (to remove excess Agrobacterium) (SIM, see Olhoft et al 2007 A novel Agrobacterium rhizogenes-mediated transformation method of soy using primary-node explants from seedlings In Vitro Cell. Dev. Biol.-Plant (2007) 43:536-549; to remove excess Agrobacterium) or Modwash medium (1× B5 major salts, 1× B5 minor salts, 1× MSIII iron, 3% Sucrose, 1× B5 vitamins, 30 mM MES, 350 mg/L Timentin pH 5.6, WO 2005/121345) and blotted dry on sterile filter paper (to prevent damage especially on the lamina) before placing on the solid SIM medium. The approximately 5 explants (Method A) or 10 to 20 (Methods B and C) explants are placed such that the target tissue was in direct contact with the medium. During the first 2 weeks, the explants could be cultured with or without selective medium. Preferably, explants are transferred onto SIM without selection for one week. For leaf explants (Method B), the explant should be placed into the medium such that it is perpendicular to the surface of the medium with the petiole imbedded into the medium and the lamina out of the medium.

For propagated axillary meristem (Method C), the explant is placed into the medium such that it is parallel to the surface of the medium (basipetal) with the explant partially embedded into the medium.

Wrap plates with Scotch 394 venting tape (3M, St. Paul, Minn., USA) are placed in a growth chamber for two weeks with a temperature averaging 25.degree. C. under 18 h light/6 h dark cycle at 70-100 ρE/m2s. The explants remains on the SIM medium with or without selection until de novo shoot growth occurred at the target area (e.g., axillary meristems at the first node above the epicotyl). Transfers to fresh medium can occur during this time. Explants are transferred from the SIM with or without selection to SIM with selection after about one week. At this time, there is considerable de novo shoot development at the base of the petiole of the leaf explants in a variety of SIM (Method B), at the primary node for seedling explants (Method A), and at the axillary nodes of propagated explants (Method C).

Preferably, all shoots formed before transformation are removed up to 2 weeks after co-cultivation to stimulate new growth from the meristems. This helped to reduce chimerism in the primary transformant and increase amplification of transgenic meristematic cells. During this time the explant may or may not be cut into smaller pieces (i.e. detaching the node from the explant by cutting the epicotyl).

3.5—Shoot Elongation

After 2 to 4 weeks (or until a mass of shoots is formed) on SIM medium (preferably with selection), the explants are transferred to SEM medium (shoot elongation medium, see Olhoft et al 2007 A novel Agrobacterium rhizogenes-mediated transformation method of soy using primary-node explants from seedlings. In Vitro Cell. Dev. Biol.-Plant (2007) 43:536-549) that stimulates shoot elongation of the shoot primordia. This medium may or may not contain a selection compound.

After every 2 to 3 weeks, the explants are transferred to fresh SEM medium (preferably containing selection) after carefully removing dead tissue. The explants should hold together and not fragment into pieces and retain somewhat healthy. The explants are continued to be transferred until the explant dies or shoots elongate. Elongated shoots >3 cm are removed and placed into RM medium for about 1 week (Methods A and B), or about 2 to 4 weeks depending on the cultivar (Method C) at which time roots began to form. In the case of explants with roots, they are transferred directly into soil. Rooted shoots are transferred to soil and hardened in a growth chamber for 2 to 3 weeks before transferring to the greenhouse. Regenerated plants obtained using this method are fertile and produced on average 500 seeds per plant.

After 5 days of co-cultivation with Agrobacterium tumefaciens transient expression of the gene of interest (GOI) is widespread on the seedling axillary meristem explants especially in the regions wounding during explant preparation (Method A). Explants are placed into shoot induction medium without selection to see how the primary-node responds to shoot induction and regeneration. Thus far, greater than 70% of the explants were formed new shoots at this region. Expression of the GOI is stable after 14 days on SIM, implying integration of the T-DNA into the soybean genome. In addition, preliminary experiments results in the formation of cDNA expressing shoots forming after 3 weeks on SIM.

For Method C, the average regeneration time of a soybean plantlet using the propagated axillary meristem protocol is 14 weeks from explant inoculation. Therefore, this method has a quick regeneration time that leads to fertile, healthy soybean plants.

### Example 4: Pathogen Assay for Soybean

4.1. Growth of Plants

T1 soy plants per event are potted and grown for 3-4 weeks in the Phytochamber (16 h-day-und 8 h-night-Rhythm at a temperature of 16° and 22° C. und a humidity of 75%) till the first 2 trifoliate leaves were fully expanded.

4.2 Inoculation

The plants are inoculated with spores of P.pachyrhizi.

In order to obtain appropriate spore material for the inoculation, soybean leaves which are infected with rust 15-20 days ago, are taken 2-3 days before the inoculation and transferred to agar plates (1% agar in H2O). The leaves are placed with their upper side onto the agar, which allowed the fungus to grow through the tissue and to produce very young spores. For the inoculation solution, the spores are knocked off the leaves and are added to a Tween-H₂O solution. The counting of spores is performed under a light microscope by means of a Thoma counting chamber. For the inoculation of the plants, the spore suspension is added into a compressed-air operated spray flask and applied uniformly onto the plants or the leaves until the leaf surface is well moisturized. For macroscopic assays a spore density of 1-5×105 spores/ml is used. For the microscopy, a density of >5×105 spores/ml is used. The inoculated plants are placed for 24 hours in a greenhouse chamber with an average of 22° C. and >90% of air humidity. The following cultivation is performed in a chamber with an average of 25° C. and 70% of air humidity.

### Example 5: Microscopical Screening

For the evaluation of the pathogen development, the inoculated leaves of plants are stained with aniline blue 48 hours after infection.

The aniline blue staining serves for the detection of fluorescent substances. During the defense reactions in host interactions and non-host interactions, substances such as phenols, callose or lignin accumulate or are produced and are incorporated at the cell wall either locally in papillae or in the whole cell (hypersensitive reaction, HR). Complexes are formed in association with aniline blue, which lead e.g. in the case of callose to yellow fluorescence. The leaf material is transferred to falcon tubes or dishes containing destaining solution II (ethanol/acetic acid 6/1) and is incubated in a water bath at 90° C. for 10-15 minutes. The destaining solution II is removed immediately thereafter, and the leaves are washed 2× with water. For the staining, the leaves are incubated for 1.5-2 hours in staining solution II (0.05% aniline blue=methyl blue, 0.067 M di-potassium hydrogen phosphate) and analyzed by microscopy immediately thereafter.

The different interaction types are evaluated (counted) by microscopy. An Olympus UV microscope BX61 (incident light) and a UV Longpath filter (excitation: 375/15, Beam splitter: 405 LP) are used. After aniline blue staining, the spores appeared blue under UV light. The papillae can be recognized beneath the fungal appressorium by a green/yellow staining. The hypersensitive reaction (HR) is characterized by a whole cell fluorescence

### Example 6: Evaluating the Susceptibility to Soybean Rust

The progression of the soybean rust disease is analyzed by imaging of the infected first trifoliate leaf (adaxial side) 14 days after infection with Phakopsora pachyrhizi. An algorithm is used to determine the percentage of the leaf area showing fungal colonies or strong yellowing/browning, which is considered as diseased leaf area (for exemplary scheme see FIG. 1).

To evaluate the resistance mediated by taLR67res or Glyma.01g238800.1G145R_V389L respectively, 3-4 week old T1 soybean plants are infected with Phakopsora pachyrhizi and cultivated as described above. All non-transgenic wild type control soy plants were grown in parallel to the transgenic plants.

At all 46 transgenic T1 soybean plants (from 4 independent events) expressing Glyma.01g238800.1G145R_V389L, 36 transgenic T1 soybean plants (from 3 independent events) expressing TaLr67res and 46 non-transgenic wild-type control plants were inoculated with spores of Phakopsora pachyrhizi. The expression of Glyma.01g238800.1G145R_V389L and TaLr67res was checked by RT-PCR.

The macroscopic disease symptoms of P. pachyrhizi on the first trifoliate leaf of the inoculated soybean plants are scored 14 days after inoculation. The average of the percentage of the leaf area showing fungal colonies or strong yellowing/browning on all leaves is considered as diseased leaf area. In this experiment the wild type control showed an average infected leaf area of 16,2%. The diseased leaf area of control was set to 100% for further analysis.

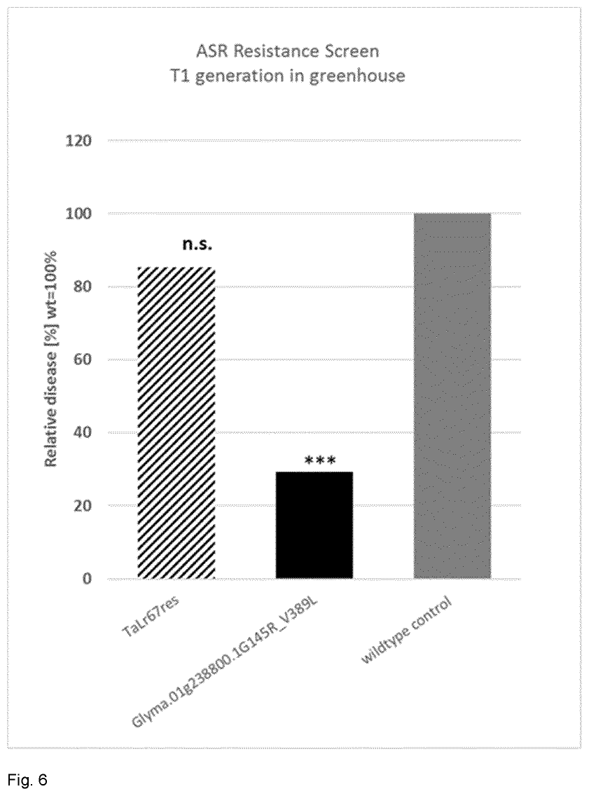

Overexpression of Glyma.01g238800.1G145R_V389L significantly (***: p<0.001) reduces the diseased leaf area in comparison to non-transgenic control plants by 70,8% (see FIG. 6). Overexpression of TaLr67res did not lead to a statistically significant reduction of disease.

### Embodiments

1. A cell having an Lr67 allele comprising one or more mutations in a soybean Lr67 gene, wherein the one or more mutations comprise, in the numbering according to SEQ ID NO. 1,
   a substitution at position G145, preferably a substitution selected from, in decreasing order of preference, G145R, G145K, G145H, G145Q, G145E, G145V, G145L and G145Y,
   and optionally also a substitution at position 1389, preferably a substitution selected from, in decreasing order of preference, 1389L, 1389W, 1389K, 1389R, 1389Q, 1389F and 1389M.
2. The cell according to embodiment 1,
   wherein the one or more mutations reduce or abolish, when expressing said Lr67 allele in a soybean plant, the susceptibility, relative to a wild type plant, to infections by a biotrophic or heminecrotrophic fungus, preferably a rust fungus, more preferably a fungus of phylum Basidiomycota, even more preferably of subphylum Pucciniomycotina, even more preferably of class Pucciniomycetes, even more preferably of order Pucciniales, even more preferably of family Chaconiaceae, Coleosporiaceae, Cronartiaceae, Melampsoraceae, Mikronegeriaceae, Phakopsoraceae, Phragmidiaceae, Pileolariaceae, Pucciniaceae, Pucciniastraceae, Pucciniosiraceae, Raveneliaceae, Sphaerophragmiaceae or Uropyxidaceae, even more preferably of genus Maravalia, Ochropsora, Olivea, Chrysomyxa, Coleosporium, Diaphanopellis, Cronartium, Endocronartium, Peridermium, Melampsora, Chrysocelis, Mikronegeria, Arthuria, Batistopsora, Cerotelium, Dasturella, Phakopsora, Prospodium, Arthuriomyces, Catenulopsora, Gerwasia, Gymnoconia, Hamaspora, Kuehneola, Phragmidium, Trachyspora, Triphragmium, Atelocauda, Pileolaria, Racospermyces, Uromycladium, Allodus, Ceratocoma, Chrysocyclus, Cumminsiella, Cystopsora, Endophyllum, Gymnosporangium, Miyagia, Puccinia, Puccorchidium, Roestelia, Sphenorchidium, Stereostratum, Uromyces, Hyalopsora, Melampsorella, Melampsoridium, Milesia, Milesina, Naohidemyces, Pucciniastrum, Thekopsora, Uredinopsis, Chardoniella, Dietelia, Pucciniosira, Diorchidium, Endoraecium, Kernkampella, Ravenelia, Sphenospora, Austropuccinia, Nyssopsora, Sphaerophragmium, Dasyspora, Leucotelium, Macruropyxis, Porotenus, Tranzschelia or Uropyxis, most preferably of species Phakopsora pachyrhizi, Phakopsora meibomiae, Puccinia graminis, Puccinia striiformis, Puccinia hordei or Puccinia recondita.
3. The cell according to embodiment 1 or 2, wherein the Lr67 gene
   a) codes for a polypeptide having an amino acid sequence of at least 40% identity to any of SEQ ID NO. 1, 2, 3, 4, 5, 6 or 7,
   or a biologically active fragment thereof, and/or
   b) comprises a nucleic acid sequence
      obtainable or obtained by amplification from the genome of Glycine max using any of the primer pairs according to SEQ ID NO. 15 and 21, 16 and 22, 17 and 23, 18 and 24, 19 and 25, or 20 and 26, and/or
      obtainable or obtained by reverse translation from an RNA hybridizing under stringent conditions to a nucleic acid probe according to SEQ ID NO. 27, 28, 29, 30, 31 or 32, and/or
      having at least 60% identity to any of SEQ ID NO. 33, 34, 35, 36, 37 or 38, and/or
   c) comprises a PF00083 Pfam domain, an InterPro domain selected from IPR020846, IPR005828, IPR036259, IPR003663 and IPR005829, a Prosite PS50850 Major facilitator superfamily (MFS) profile and/or 12 transmembrane domains.
4. The cell according to any of embodiments 1-3, wherein the Lr67 allele is a hypomorphic allele, an amorphic allele, a neomorphic allele or an antimorphic allele, preferably a dominant-negative allele.
5. The cell according to any of embodiments 1-4, wherein the Lr67 allele codes for a polypeptide differing from any of SEQ ID NO. 1, 2, 3, 4, 5, 6 or 7 only
   a) by one or more conservative amino acid substitutions and/or
   b) by one or more mutations according to table 1.
6. The cell according to any of embodiments 1-5, wherein the Lr67 allele is integral in the genome of the cell.
7. The cell according to any of embodiments 1-6, wherein the cell is homozygous for the Lr67 gene or heterozygous for the Lr67 gene.
8. The cell according to any of embodiments 1-7, wherein the cell, when in meiosis, is non-segregating or segregating for the Lr67 allele.
9. The cell according to any of embodiments 1-8, wherein the Lr67 allele is operably linked to a heterologous promoter, preferably a rust-inducible promoter.
10. The cell according to any of embodiments 1-9, wherein the cell contains a negligibly low content of, and preferably none of, allergens and/or anti-metabolites.
11. The cell according to any of embodiments 1-10, wherein the cell is a plant or yeast cell, preferably a monocotyledon or dicotyledon plant, more preferably a plant of order Fabales, more preferably a plant of family Fabaceae, more preferably a plant of tribus Phaseoleae, more preferably of genus Glycine, most preferably of species Glycine max, Glycine soja, Glycine gracilis or a cross Glycine max×Glycine soja.
12. The plant cell according to embodiment 11, wherein the cell is a transgenic plant cell or wherein the mutation in the Lr67 gene is an artificially induced heritable allele.
13. An expression construct comprising
   an Lr67 allele comprising one or more mutations in a soybean Lr67 gene as defined in any of embodiments 1-12,
   wherein the Lr67 allele is operably linked to a heterologous polynucleotide.
14. The expression construct according to embodiment 13, wherein the heterologous polynucleotide comprises a promoter for expressing the Lr67 allele in a plant leaf cell, preferably a soybean plant leaf cell.
15. A plant or plant part comprising a cell according to any of embodiments 1-12 or transformed with an expression construct according to any of embodiments 13-14.
16. The plant or plant part according to embodiment 15, having reduced or abolished susceptibility, relative to a wild type plant, to infections by a biotrophic or heminecrotrophic fungus, preferably a rust fungus, more preferably a fungus of phylum Basidiomycota, even more preferably of subphylum Pucciniomycotina, even more preferably of class Pucciniomycetes, even more preferably of order Pucciniales, even more preferably of family Chaconiaceae, Coleosporiaceae, Cronartiaceae, Melampsoraceae, Mikronegeriaceae, Phakopsoraceae, Phragmidiaceae, Pileolariaceae, Pucciniaceae, Pucciniastraceae, Pucciniosiraceae, Raveneliaceae, Sphaerophragmiaceae or Uropyxidaceae, even more preferably of genus Maravalia, Ochropsora, Olivea, Chrysomyxa, Coleosporium, Diaphanopellis, Cronartium, Endocronartium, Peridermium, Melampsora, Chrysocelis, Mikronegeria, Arthuria, Batistopsora, Cerotelium, Dasturella, Phakopsora, Prospodium, Arthuriomyces, Catenulopsora, Gerwasia, Gymnoconia, Hamaspora, Kuehneola, Phragmidium, Trachyspora, Triphragmium, Atelocauda, Pileolaria, Racospermyces, Uromycladium, Allodus, Ceratocoma, Chrysocyclus, Cumminsiella, Cystopsora, Endophyllum, Gymnosporangium, Miyagia, Puccinia, Puccorchidium, Roestelia, Sphenorchidium, Stereostratum, Uromyces, Hyalopsora, Melampsorella, Melampsoridium, Milesia, Milesina, Naohidemyces, Pucciniastrum, Thekopsora, Uredinopsis, Chardoniella, Dietelia, Pucciniosira, Diorchidium, Endoraecium, Kernkampella, Ravenelia, Sphenospora, Austropuccinia, Nyssopsora, Sphaerophragmium, Dasyspora, Leucotelium, Macruropyxis, Porotenus, Tranzschelia or Uropyxis, most preferably of species Phakopsora pachyrhizi, Phakopsora meibomiae, Puccinia graminis, Puccinia striiformis, Puccinia hordei or Puccinia recondita.
17. The plant or plant part according to any of embodiments 15-16,
   i) wherein the Lr67 allele is integral in the genome of the plant or plant part, and/or
   ii) wherein the plant or plant part is homozygous for the Lr67 gene or heterozygous for the Lr67 gene, and/or
   iii) wherein the plant or plant part, when in meiosis, is non-segregating or segregating for the Lr67 allele, and/or
   iv) wherein the Lr67 allele is operably linked to a heterologous promoter, and/or
   v) wherein the Lr67 allele is, in the genome of the plant or plant part, integrated at a different locus than the corresponding wild type Lr67 gene, and/or
   vi) wherein the plant or plant part is transgenic or wherein the mutation in the Lr67 gene is an artificially induced heritable allele.
18. The plant or plant part according to any of embodiments 15-17, wherein the plant is or the part thereof belongs to a monocotyledon or dicotyledon plant, more preferably a plant of order Fabales, more preferably a plant of family Fabaceae, more preferably a plant of tribus Phaseoleae, more preferably of genus Glycine, most preferably of species Glycine max, Glycine soja, Glycine gracilis or a cross Glycine max×Glycine soja.
19. An ensemble of at least 50 plants according to any of embodiments 15-18.
20. The ensemble according to embodiment 19, wherein the plants are growing in a field or greenhouse.
21. A seed, flower, leaf, fruit, processed food, or food ingredient from the plant according to any of embodiments 15-18.
22. An accumulation of at least 1 kg of seeds according to embodiment 21.
23. Use of a plant according to any of embodiments 15-18 or of an ensemble according to any of embodiments 19-20, or a part of such plant or ensemble, as animal feed or to produce a feed product for animal or a food product for human consumption.
24. Use according to embodiment 23, wherein the food product is a wholemeal, meal or starch, or wherein the food product is a fermentation product of the part of the plant or ensemble, preferably a fermentation product of seed.
25. A method for creating a cell according to any of embodiments 1-12, comprising the step of transforming a cell with a nucleic acid coding for an Lr67 allele according to any of embodiments 1-12.
26. A method for creating a cell according to any of embodiments 1-12, comprising the steps of
   i) introducing into a cell an enzyme having endonuclease or nickase activity and recognizing a soybean Lr67 gene sequence,
   ii) effecting by action of said enzyme a change in nucleotide sequence such as to introduce an amino acid change at position 145, using the numbering according to SEQ ID NO. 1.
27. The method according to embodiment 26, wherein the enzyme is functionally linked to a mutation inducing agent, preferably a deaminase, glycosylase or a chemical agent, preferably acridine, psoralen, and/or ethidium bromide.
28. The method according to embodiment 26 or 27, further comprising the steps of growing a plant from said cell and selecting, in comparison to a wild type plant, plants having reduced or abolished susceptibility to infections by a biotrophic or heminecrotrophic fungus, preferably a rust fungus, more preferably a fungus of phylum Basidiomycota, even more preferably of subphylum Pucciniomycotina, even more preferably of class Pucciniomycetes, even more preferably of order Pucciniales, even more preferably of family Chaconiaceae, Coleosporiaceae, Cronartiaceae, Melampsoraceae, Mikronegeriaceae, Phakopsoraceae, Phragmidiaceae, Pileolariaceae, Pucciniaceae, Pucciniastraceae, Pucciniosiraceae, Raveneliaceae, Sphaerophragmiaceae or Uropyxidaceae, even more preferably of genus Maravalia, Ochropsora, Olivea, Chrysomyxa, Coleosporium, Diaphanopellis, Cronartium, Endocronartium, Peridermium, Melampsora, Chrysocelis, Mikronegeria, Arthuria, Batistopsora, Cerotelium, Dasturella, Phakopsora, Prospodium, Arthuriomyces, Catenulopsora, Gerwasia, Gymnoconia, Hamaspora, Kuehneola, Phragmidium, Trachyspora, Triphragmium, Atelocauda, Pileolaria, Racospermyces, Uromycladium, Allodus, Ceratocoma, Chrysocyclus, Cumminsiella, Cystopsora, Endophyllum, Gymnosporangium, Miyagia, Puccinia, Puccorchidium, Roestelia, Sphenorchidium, Stereostratum, Uromyces, Hyalopsora, Melampsorella, Melampsoridium, Milesia, Milesina, Naohidemyces, Pucciniastrum, Thekopsora, Uredinopsis, Chardoniella, Dietelia, Pucciniosira, Diorchidium, Endoraecium, Kernkampella, Ravenelia, Sphenospora, Austropuccinia, Nyssopsora, Sphaerophragmium, Dasyspora, Leucotelium, Macruropyxis, Porotenus, Tranzschelia or Uropyxis, most preferably of species Phakopsora pachyrhizi, Phakopsora meibomiae, Puccinia graminis, Puccinia striiformis, Puccinia hordei or Puccinia recondita.
29. A method for producing a population of plants each having an enhanced resistance to at least one biotrophic or heminecrotrophic fungus, preferably a rust fungus, comprising the steps of
   i) providing a plant according to any of embodiments 15-18, and
   ii) crossing the plant of step a) with a compatible plant without active Lr67 allele,
   iii) growing progeny obtained by the crossing of step b).
30. The method for producing a population according to embodiment 29, wherein the plant in step a) is homozygous for the Lr67 allele.
31. A method for reducing or abolishing susceptibility of a plant or plant part, in comparison to a wild type plant, to infections by a biotrophic or heminecrotrophic fungus, preferably a rust fungus, comprising causing or increasing expression or activity of an Lr67 allele comprising one or more mutations in a soybean Lr67 gene, wherein the one or more mutations comprise, in the numbering according to SEQ ID NO. 1
   a substitution at position G145, preferably a substitution selected from, in decreasing order of preference, G145R, G145K, G145H, G145Q, G145E, G145V, G145L and G145Y,
   and optionally also a substitution at position 1389, preferably a substitution selected from, in decreasing order of preference, 1389L, 1389W, 1389K, 1389R, 1389Q, 1389F and 1389M.
32. The method according to embodiment 31,
   wherein the fungus is a fungus of phylum Basidiomycota, even more preferably of subphylum Pucciniomycotina, even more preferably of class Pucciniomycetes, even more preferably of order Pucciniales, even more preferably of family Chaconiaceae, Coleosporiaceae, Cronartiaceae, Melampsoraceae, Mikronegeriaceae, Phakopsoraceae, Phragmidiaceae, Pileolariaceae, Pucciniaceae, Pucciniastraceae, Pucciniosiraceae, Raveneliaceae, Sphaerophragmiaceae or Uropyxidaceae, even more preferably of genus Maravalia, Ochropsora, Olivea, Chrysomyxa, Coleosporium, Diaphanopellis, Cronartium, Endocronartium, Peridermium, Melampsora, Chrysocelis, Mikronegeria, Arthuria, Batistopsora, Cerotelium, Dasturella, Phakopsora, Prospodium, Arthuriomyces, Catenulopsora, Gerwasia, Gymnoconia, Hamaspora, Kuehneola, Phragmidium, Trachyspora, Triphragmium, Atelocauda, Pileolaria, Racospermyces, Uromycladium, Allodus, Ceratocoma, Chrysocyclus, Cumminsiella, Cystopsora, Endophyllum, Gymnosporangium, Miyagia, Puccinia, Puccorchidium, Roestelia, Sphenorchidium, Stereostratum, Uromyces, Hyalopsora, Melampsorella, Melampsoridium, Milesia, Milesina, Naohidemyces, Pucciniastrum, Thekopsora, Uredinopsis, Chardoniella, Dietelia, Pucciniosira, Diorchidium, Endoraecium, Kernkampella, Ravenelia, Sphenospora, Austropuccinia, Nyssopsora, Sphaerophragmium, Dasyspora, Leucotelium, Macruropyxis, Porotenus, Tranzschelia or Uropyxis, most preferably of species Phakopsora pachyrhizi, Puccinia graminis, Puccinia striiformis, Puccinia hordei or Puccinia recondita.
33. The method according to any of embodiments 31-32, wherein the Lr67 gene
   a) codes for a polypeptide having an amino acid sequence of at least 40% identity to any of SEQ ID NO. 1, 2, 3, 4, 5, 6 or 7,
   or a biologically active fragment thereof, and/or
   b) comprises a nucleic acid sequence
      obtainable or obtained by amplification from the genome of Glycine max using any of the primer pairs according to SEQ ID NO. 15 and 21, 16 and 22, 17 and 23, 18 and 24, 19 and 25, or 20 and 26, and/or
      obtainable or obtained by reverse translation from an RNA hybridizing under stringent conditions to a nucleic acid probe according to SEQ ID NO. 27, 28, 29, 30, 31 or 32, and/or
      having at least 60% identity to any of SEQ ID NO. 33, 34, 35, 36, 37 or 38, and/or
   c) comprises a PF00083 Pfam domain, an InterPro domain selected from IPR020846, IPR005828, IPR036259, IPR003663 and IPR005829, a Prosite PS50850 Major facilitator superfamily (MFS) profile and/or 12 transmembrane domains.
34. The method according to any of embodiments 31-33, wherein the Lr67 gene
   a) codes for a polypeptide having an amino acid sequence of at least 40% identity to any of SEQ ID NO. 1, 2, 3, 4, 5, 6 or 7,
   or a biologically active fragment thereof, and/or
   b) comprises a nucleic acid sequence
      obtainable or obtained by amplification from the genome of Glycine max using any of the primer pairs according to SEQ ID NO. 15 and 21, 16 and 22, 17 and 23, 18 and 24, 19 and 25, or 20 and 26, and/or
      obtainable or obtained by reverse translation from an RNA hybridizing under stringent conditions to a nucleic acid probe according to SEQ ID NO. 27, 28, 29, 30, 31 or 32, and/or
      having at least 60% identity to any of SEQ ID NO. 33, 34, 35, 36, 37 or 38, and/or
   c) comprises a PF00083 Pfam domain, an InterPro domain selected from IPR020846, IPR005828, IPR036259, IPR003663 and IPR005829, a Prosite PS50850 Major facilitator superfamily (MFS) profile and/or 12 transmembrane domains.
35. The method according to any of embodiments 31-34, wherein the plant or plant part is transgenic plant or wherein the mutation in the Lr67 gene is an artificially induced heritable allele.
36. The method according to any of embodiments 31-35,
   i) wherein the Lr67 allele is integral in the genome of the plant or plant part, and/or
   ii) wherein the plant or plant part is homozygous for the Lr67 gene or heterozygous for the Lr67 gene, and/or
   iii) wherein the plant or plant part, when in meiosis, is non-segregating or segregating for the Lr67 allele, and/or
   iv) wherein the Lr67 allele is operably linked to a heterologous polynucleotide, preferably a heterologous promoter, and/or preferably wherein the heterologous polynucleotide comprises a promoter for expressing the Lr67 allele in a plant leaf cell, preferably a soybean plant leaf cell.
   v) wherein the Lr67 allele is, in the genome of the plant or plant part, integrated at a different locus than the corresponding wild type Lr67 gene, and/or
   vi) wherein the plant or plant part is transgenic or wherein the mutation in the Lr67 gene is an artificially induced heritable allele, and/or
   vii) wherein the plant is or the part thereof belongs to a monocotyledon or dicotyledon plant, more preferably a plant of order Fabales, more preferably a plant of family Fabaceae, more preferably a plant of tribus Phaseoleae, more preferably of genus Glycine, most preferably of species Glycine max, Glycine soja, Glycine gracilis or a cross Glycine max×Glycine soja.
37. A method of assaying a plant for resistance to a biotrophic or heminecrotrophic fungus, preferably a rust fungus, comprising the screening for the presence of the Lr67 allele in a cell of said plant,
   preferably comprising the detection of a polynucleotide, preferably an mRNA,
   a) hybridizing under stringent conditions to a nucleic acid probe according to SEQ ID NO. 27, 28, 29, 30, 31 or 32, and/or
   b) having at least 60% identity to any of SEQ ID NO. 33, 34, 35, 36, 37 or 38.
38. Method of propagation of a sexually reproducing plant, comprising:
   i) obtaining a plurality of seeds of a plant, preferably a monocotyledon or dicotyledon plant, more preferably a plant of order Fabales, more preferably a plant of family Fabaceae, more preferably a plant of tribus Phaseoleae, more preferably of genus Glycine, most preferably of species Glycine max, Glycine soja, Glycine gracilis or a cross Glycine max×Glycine soja,
   ii) ascertaining that at least about 25%, more preferably at least 50%, even more preferably 25%-95% and most preferably 45%-100% of the seed of one species comprise an Lr67 allele according to any of embodiments 1 to 12, and
   iii) planting seed of the plurality of seeds satisfying the condition according to step ii).

1. A cell having an Lr67 allele comprising one or more mutations in a soybean Lr67 gene,
wherein the one or more mutations comprise, in the numbering according to SEQ ID NO. 1,
a substitution at position G145,
and optionally also a substitution at position 1389.

2. The cell according to claim 1,
wherein the one or more mutations reduce or abolish, when expressing said Lr67 allele in a soybean plant, the susceptibility, relative to a wild type plant, to infections by a biotrophic or heminecrotrophic fungus.

3. The cell according to claim 1,
wherein the Lr67 gene
a) codes for a polypeptide having an amino acid sequence of at least 40% identity to any of SEQ ID NO. 1, 2, 3, 4, 5, 6 or 7,
or a biologically active fragment thereof, and/or
b) comprises a nucleic acid sequence
obtainable or obtained by amplification from the genome of Glycine max using any of the primer pairs according to SEQ ID NO. 15 and 21, 16 and 22, 17 and 23, 18 and 24, 19 and 25, or 20 and 26, and/or
obtainable or obtained by reverse translation from an RNA hybridizing under stringent conditions to a nucleic acid probe according to SEQ ID NO. 27, 28, 29, 30, 31 or 32, and/or
having at least 60% identity to any of SEQ ID NO. 33, 34, 35, 36, 37 or 38, and/or
c) comprises a PF00083 Pfam domain, an InterPro domain selected from IPR020846, IPR005828, IPR036259, IPR003663 and IPR005829, a Prosite PS50850 Major facilitator superfamily (MFS) profile and/or 12 transmembrane domains.

4. The cell according to claim 1,
wherein the Lr67 allele codes for a polypeptide differing from any of SEQ ID NO. 1, 2, 3, 4, 5, 6 or 7 only
a) by one or more conservative amino acid substitutions and/or
b) by one or more mutations according to table 1.

5. The cell according to claim 1,
wherein the Lr67 allele is a hypomorphic allele, an amorphic allele, a neomorphic allele or an antimorphic allele; and/or
wherein the Lr67 allele is integral in the genome of the cell; and/or
wherein the cell is homozygous for the Lr67 gene or heterozygous for the Lr67 gene; and/or
wherein the cell, when in meiosis, is non-segregating or segregating for the Lr67 allele; and/or
wherein the Lr67 allele is operably linked to a heterologous promoter; and/or
wherein the cell contains a negligibly low content of allergens and/or anti-metabolites.

6. The cell according to claim 1,
wherein the cell is a plant or yeast cell.

7. The plant cell according to claim 6,
wherein the cell is a transgenic plant cell or wherein the mutation in the Lr67 gene is an artificially induced heritable allele.

8. An expression construct comprising
an Lr67 allele comprising one or more mutations in a soybean Lr67 gene as defined in claim 1,
wherein the Lr67 allele is operably linked to a heterologous polynucleotide.

9. The expression construct according to claim 8,
wherein the heterologous polynucleotide comprises a promoter for expressing the Lr67 allele in a plant leaf cell.

10. A plant or plant part comprising a cell according to claim 1,
having reduced or abolished susceptibility, relative to a wild type plant, to infections by a biotrophic or heminecrotrophic fungus.

11. The plant or plant part according to claim 10,
i) wherein the Lr67 allele is integral in the genome of the plant or plant part, and/or
ii) wherein the plant or plant part is homozygous for the Lr67 gene or heterozygous for the Lr67 gene, and/or
iii) wherein the plant or plant part, when in meiosis, is non-segregating or segregating for the Lr67 allele, and/or
iv) wherein the Lr67 allele is operably linked to a heterologous promoter, and/or
v) wherein the Lr67 allele is, in the genome of the plant or plant part, integrated at a different locus than the corresponding wild type Lr67 gene, and/or
vi) wherein the plant or plant part is transgenic or wherein the mutation in the Lr67 gene is an artificially induced heritable allele.

12. The plant or plant part according to claim 10,
wherein the plant is or the part thereof belongs to a monocotyledon or dicotyledon plant.

13. An ensemble of at least 50 plants according to claim 10.

14. A seed, flower, leaf, fruit, processed food, or food ingredient from the plant according to claim 10.

15. An accumulation of at least 1 kg of seeds according to claim 14.

16. (canceled)

17. A method for creating a cell, comprising the step of transforming a cell with a nucleic acid coding for an Lr67 allele comprising one or more mutations in a soybean Lr67 gene,
wherein the one or more mutations comprise, in the numbering according to SEQ ID NO. 1,
a substitution at position G145,
and optionally also a substitution at position I389.

18. A method for creating a cell according to claim 1, comprising the steps of
i) introducing into a cell an enzyme having endonuclease or nickase activity and recognizing a soybean Lr67 gene sequence,
ii) effecting by action of said enzyme a change in nucleotide sequence such as to introduce an amino acid change at position G145 and optionally at position 1389, using the numbering according to SEQ ID NO. 1.

19. The method according to claim 18, further comprising the steps of growing a plant from said cell and selecting, in comparison to a wild type plant, plants having reduced or abolished susceptibility to infections by a biotrophic or heminecrotrophic fungus.

20. A method for producing a population of plants each having an enhanced resistance to at least one biotrophic or heminecrotrophic fungus, comprising the steps of
i) providing a plant according to claim 10, and
ii) crossing the plant of step a) with a compatible plant without active Lr67 allele, and
iii) growing progeny obtained by the crossing of step b).

21. A method for reducing or abolishing susceptibility of a plant or plant part, in comparison to a wild type plant, to infections by a biotrophic or heminecrotrophic fungus, comprising causing or increasing expression or activity of an Lr67 allele comprising one or more mutations in a soybean Lr67 gene,
wherein the one or more mutations comprise, in the numbering according to SEQ ID NO. 1,
a substitution at position G145,
and optionally also a substitution at position 1389.

22. The method according to claim 21,
wherein the fungus is a fungus of phylum Basidiomycota.

23. The method according to claim 21,
wherein the Lr67 gene
a) codes for a polypeptide having an amino acid sequence of at least 40% identity to any of SEQ ID NO. 1, 2, 3, 4, 5, 6 or 7,
or a biologically active fragment thereof, and/or
b) comprises a nucleic acid sequence
obtainable or obtained by amplification from the genome of Glycine max using any of the primer pairs according to SEQ ID NO. 15 and 21, 16 and 22, 17 and 23, 18 and 24, 19 and 25, or 20 and 26, and/or
obtainable or obtained by reverse translation from an RNA hybridizing under stringent conditions to a nucleic acid probe according to SEQ ID NO. 27, 28, 29, 30, 31 or 32, and/or
having at least 60% identity to any of SEQ ID NO. 33, 34, 35, 36, 37 or 38, and/or
c) comprises a PF00083 Pfam domain, an InterPro domain selected from IPR020846, IPR005828, IPR036259, IPR003663 and IPR005829, a Prosite PS50850 Major facilitator superfamily (MFS) profile and/or 12 transmembrane domains.

24. The method according to claim 21,
wherein the Lr67 gene
a) codes for a polypeptide having an amino acid sequence of at least 40% identity to any of SEQ ID NO. 1, 2, 3, 4, 5, 6 or 7,
or a biologically active fragment thereof, and/or
b) comprises a nucleic acid sequence
obtainable or obtained by amplification from the genome of Glycine max using any of the primer pairs according to SEQ ID NO. 15 and 21, 16 and 22, 17 and 23, 18 and 24, 19 and 25, or 20 and 26, and/or
obtainable or obtained by reverse translation from an RNA hybridizing under stringent conditions to a nucleic acid probe according to SEQ ID NO. 27, 28, 29, 30, 31 or 32, and/or
having at least 60% identity to any of SEQ ID NO. 33, 34, 35, 36, 37 or 38, and/or
c) comprises a PF00083 Pfam domain, an InterPro domain selected from IPR020846, IPR005828, IPR036259, IPR003663 and IPR005829, a Prosite PS50850 Major facilitator superfamily (MFS) profile and/or 12 transmembrane domains.

25. The method according to claim 21,
wherein the plant or plant part is transgenic plant or wherein the mutation in the Lr67 gene is an artificially induced heritable allele.

26. The method according to claim 21,
i) wherein the Lr67 allele is integral in the genome of the plant or plant part, and/or
ii) wherein the plant or plant part is homozygous for the Lr67 gene or heterozygous for the Lr67 gene, and/or
iii) wherein the plant or plant part, when in meiosis, is non-segregating or segregating for the Lr67 allele, and/or
iv) wherein the Lr67 allele is operably linked to a heterologous polynucleotide,
v) wherein the Lr67 allele is, in the genome of the plant or plant part, integrated at a different locus than the corresponding wild type Lr67 gene, and/or
vi) wherein the plant or plant part is transgenic or wherein the mutation in the Lr67 gene is an artificially induced heritable allele, and/or
vii) wherein the plant is or the part thereof belongs to a monocotyledon or dicotyledon plant.

27. A method of assaying a plant for resistance to a biotrophic or heminecrotrophic fungus, comprising the screening for the presence of the Lr67 allele in a cell of said plant,
comprising the detection of a polynucleotide
a) hybridizing under stringent conditions to a nucleic acid probe according to SEQ ID NO. 27, 28, 29, 30, 31 or 32, and/or
b) having at least 60% identity to any of SEQ ID NO. 33, 34, 35, 36, 37 or 38.

28. Method of propagation of a sexually reproducing plant, comprising:
i) obtaining a plurality of seeds of a plant,
ii) ascertaining that at least about 25% of the seed of one species comprise an Lr67 allele according to claim 1, and
iii) planting seed of the plurality of seeds satisfying the condition according to step ii).


SEQUENCE LISTING (38 sequences)

SEQ ID NO: 1 - protein, 522 aa, Artificial Sequence
  Artificial sequence for sequence alignments
MAGGGFTNAGGGGDFEAKITPIVIISCIMAATGGLMFGYDIGVSGGVTSMPPFLKKFFPTVYRKTVEEKGLDSNYCKYDNQGLQLFTSSLYLAGLTSTFFASYTTRRLGRRLTMLIAGFFFIAGVVLNAAAQDLAMLIVGRILLGCGVGFANQAVPVFLSEIAPSRIRGALNILFQLNITIGILFANLVNYGTNKIKGGWGWRLSLGLAGIPAVLLTLGALFVVDTPNSLIERGRLEEGKTVLKKIRGTDNIELEFQELLEASRVAKEVKHPFRNLLKRRNRPQLVISIALQIFQQFTGINAIMFYAPVLFNTLGFKNDASLYSAVITGAVNVLSTVVSIYSVDKLGRRMLLLEAGVQMFLSQVVIAIILGIKVTDHSDDLSKGIAILVVVMVCTFVSSFAWSWGPLGWLIPSETFPLETRSAGQSVTVCVNLLFTFVIAQAFLSMLCHFKFGIFLFFSGWVLVMSVFVLFLLPETKNVPIEEMTERVWKQHWFWKRFIDDAADEKVANVSNGNGFDPTSRL

SEQ ID NO: 2 - protein, 522 aa, Glycine max
  Soybean wild type Lr67 gene Glyma.01G238800.1.Wm82.a2.v1
MAGGGFTSGGGAGDFEAKITPIVIISCIMAATGGLMFGYDVGVSGGVTSMPPFLKKFFPTVYRKTVEEKGLDSNYCKYDNQGLQLFTSSLYLAGLTSTFFASYTTRRLGRRLTMLIAGVFFICGVVLNAAAQDLAMLIVGRILLGCGVGFANQAVPVFLSEIAPSRIRGALNILFQLNVTIGILFANLVNYGTNKIKGGWGWRLSLGLAGIPAVLLTLGALFVVDTPNSLIERGRLEEGKTVLKKIRGTDNIELEFQELLEASRVAKEVKHPFRNLLKRRNRPQLVISVALQIFQQFTGINAIMFYAPVLFNTLGFKNDASLYSAVITGAVNVLSTVVSIYSVDKVGRRILLLEAGVQMFLSQVVIAIILGIKVTDHSDDLSKGIAILVVVMVCTFVSSFAWSWGPLGWLIPSETFPLETRSAGQSVTVCVNLLFTFVIAQAFLSMLCHFKFGIFLFFSGWVLVMSVFVLFLLPETKNVPIEEMTERVWKQHWFWKRFIDDAADEKVAHVSNGNGFDPTSRL

SEQ ID NO: 3 - protein, 522 aa, Glycine max
  Soybean wild type Lr67 gene Glyma.11G004800.1.Wm82.a2.v1
MAVGGFTNAAGGADFEAKITPIVIISCIMAATGGLMFGYDVGVSGGVTSMPPFLKKFFPTVYRKTVEEKGLDSNYCKYDNQGLQLFTSSLYLAGLTSTFFASYTTRRLGRRLTMLIAGFFFIGGVVLNAAAQDLAMLIVGRILLGCGVGFANQAVPVFLSEIAPSRIRGALNILFQLNVTIGILFANLVNYGTNKIKGGWGWRLSLGLAGIPAVLLTLGALFVVDTPNSLIERGRLEEGKTVLKKIRGTDNIELEFQELVEASRVAKEVKHPFRNLLKRRNRPQLVISIALQIFQQFTGINAIMFYAPVLFNTLGFKNDASLYSAVITGAVNVLSTVVSIYSVDKLGRRMLLLEAGVQMFLSQVVIAIILGIKVTDHSDDLSKGIAILVVVMVCTFVSSFAWSWGPLGWLIPSETFPLETRSAGQSVTVCVNLLFTFVIAQAFLSMLCHFKFGIFLFFSGWVLVMSVFVLFLLPETKNVPIEEMTERVWKQHWFWKRFIDDAADEKVANVSNGNGFDPTSRL

SEQ ID NO: 4 - protein, 473 aa, Glycine max
  Soybean wild type Lr67 gene Glyma.11G004800.2.Wm82.a2.v1
MPPFLKKFFPTVYRKTVEEKGLDSNYCKYDNQGLQLFTSSLYLAGLTSTFFASYTTRRLGRRLTMLIAGFFFIGGVVLNAAAQDLAMLIVGRILLGCGVGFANQAVPVFLSEIAPSRIRGALNILFQLNVTIGILFANLVNYGTNKIKGGWGWRLSLGLAGIPAVLLTLGALFVVDTPNSLIERGRLEEGKTVLKKIRGTDNIELEFQELVEASRVAKEVKHPFRNLLKRRNRPQLVISIALQIFQQFTGINAIMFYAPVLFNTLGFKNDASLYSAVITGAVNVLSTVVSIYSVDKLGRRMLLLEAGVQMFLSQVVIAIILGIKVTDHSDDLSKGIAILVVVMVCTFVSSFAWSWGPLGWLIPSETFPLETRSAGQSVTVCVNLLFTFVIAQAFLSMLCHFKFGIFLFFSGWVLVMSVFVLFLLPETKNVPIEEMTERVWKQHWFWKRFIDDAADEKVANVSNGNGFDPTSRL

SEQ ID NO: 5 - protein, 475 aa, Glycine max
  Soybean wild type Lr67 gene Glyma.10G249500.1.Wm82.a2.v1
MAGGGEFEAKITPIVILSCMMAATGGLMFGYDIGVSGGVTSMPAFLKEFFPEVYRKTVEEEELDSNYCKYDNEKLQLFTSCLYLAGLIATFFASHITRRQGRRATMLISGFIFIAGVAFNAAAQNLAMLIIGRVLLGSGVGFANQAVPVFLSEIAPSQIRGALNILFQLNITLGILFSNLVNYATNKIKGGWGWRLSLGLGGLPALLLTLGAFMVVDTPNSLIERGHLEEGKVVLRKIRGIDNIEPEFLELLHASRVAKEVKHPFRNILKRKNRPQLVICIALQIFQQFTGINAIMFYAPVLFNTLGFKNDASLYSAVIIGAVNVVSTVVSIYSVDRLGRRILLLEAGVQMFLSQLVIAVIIGMKVKDHSEDLSKGYAVLVVVLVCIFVSAFAWSWGPLSWLIPSEIFPLETRSAGQSIAVCVNLLCTFVIAQAFLSMLCFFKFGIFLFFSGCVLLMSTFVLFLLPETKNVPLKR

SEQ ID NO: 6 - protein, 512 aa, Glycine max
  Soybean wild type Lr67 gene Glyma.20G144300.1.Wm82.a2.v1
MAGGGFTTSGGEFEAKITPIVILSCMMAATGGLMFGYDIGVSGGVTSMPAFLKEFFPEVYRKTVEEEELDSNYCKYDNEKLQLFTSCLYLAGLMATFLASHITRRQGRRATMLISGFIFIAGVAFNAAAQNLAMLIIGRVLLGSGVGFANQAVPVFLSEIAPSRIRGALNILFQLNITLGILFSNLVNYATNKIKGGWGWRLSLGLGGLPALLLTLGAFLVVDTPNSLIERGHLEEGKSVLRKIRGIDNIEPEFLELLDASRVAKEVKHPFRNILKRKNRPQLVISIALQIFQQFTGINAIMFYAPVLFNTLGFKNDASLYSAVITGAVNVVSTVVSIYSVDRLGRKMLLLEAGAQMFLSQLVIAVIIGMKVKDHSEDLSKGFAVLVVVLVCIFVSAFAWSWGPLSWLIPSEIFPLETRSAGQSIAVCVNLLCTFVIAQAFLSMLCFFKFGIFLFFSGCVLIMSTFVLLLLPETKNVPIEEMTERVWKQHWLWNRFIDEDDCVKEEKVVTGN

SEQ ID NO: 7 - protein, 500 aa, Glycine max
  Soybean wild type Lr67 gene Glyma.10G249400.1.Wm82.a2.v1
MAGGGFVSASGESHFEAKITFAVIISCIMAATGGLMFGYDIGISGGVTSMPSFLEKFFPEVYRKIQDHGVDSNYCKYDNQTLQLFTSSLYLAALVATMFASSVTRTLGRKQTMLIAGIFFIVGTVLNAVANSLLLLIVGRILLGCGVGFANQAVPVFISEIAPTRIRGALNIMFQLNITIGILIANIVNYFTAKIEGGYGWRISVALAGIPAIMLTFGSLLVHDTPNSLIERGLEDEGKAVLKKIRGVENVEPEFQEILKASKVAKAVKNPFQNLLKRHNRPPLIIAVMMQVFQQFTGINAIMFYAPVLFSTLGFKSDASLYSAVITGAVNVLSTLVSVYFVDKAGRRMLLLEACVQMFVSQMVIGTVLGLKVQDHSDSLNKGLGVLVVVMVCTFVASFAWSWGPLGWLIPSETFPLEARSAGQSVTVFTNMLFTFIIAQGFLSMMCHLKFGIFFFFSAWVLAMAIFTVLLIPETKNIPIEEMTDKVWRNHWFWKSYMED

SEQ ID NO: 8 - protein, 522 aa, Artificial Sequence
  Lr67 allele based on SEQ ID NO. 1
MAGGGFTNAGGGGDFEAKITPIVIISCIMAATGGLMFGYDIGVSGGVTSMPPFLKKFFPTVYRKTVEEKGLDSNYCKYDNQGLQLFTSSLYLAGLTSTFFASYTTRRLGRRLTMLIAGFFFIAGVVLNAAAQDLAMLIVGRILLRCGVGFANQAVPVFLSEIAPSRIRGALNILFQLNITIGILFANLVNYGTNKIKGGWGWRLSLGLAGIPAVLLTLGALFVVDTPNSLIERGRLEEGKTVLKKIRGTDNIELEFQELLEASRVAKEVKHPFRNLLKRRNRPQLVISIALQIFQQFTGINAIMFYAPVLFNTLGFKNDASLYSAVITGAVNVLSTVVSIYSVDKLGRRMLLLEAGVQMFLSQVVIAIILGIKVTDHSDDLSKGIAILLVVMVCTFVSSFAWSWGPLGWLIPSETFPLETRSAGQSVTVCVNLLFTFVIAQAFLSMLCHFKFGIFLFFSGWVLVMSVFVLFLLPETKNVPIEEMTERVWKQHWFWKRFIDDAADEKVANVSNGNGFDPTSRL

SEQ ID NO: 9 - protein, 522 aa, Artificial Sequence
  Lr67 allele based on SEQ ID NO. 2
MAGGGFTSGGGAGDFEAKITPIVIISCIMAATGGLMFGYDVGVSGGVTSMPPFLKKFFPTVYRKTVEEKGLDSNYCKYDNQGLQLFTSSLYLAGLTSTFFASYTTRRLGRRLTMLIAGVFFICGVVLNAAAQDLAMLIVGRILLRCGVGFANQAVPVFLSEIAPSRIRGALNILFQLNVTIGILFANLVNYGTNKIKGGWGWRLSLGLAGIPAVLLTLGALFVVDTPNSLIERGRLEEGKTVLKKIRGTDNIELEFQELLEASRVAKEVKHPFRNLLKRRNRPQLVISVALQIFQQFTGINAIMFYAPVLFNTLGFKNDASLYSAVITGAVNVLSTVVSIYSVDKVGRRILLLEAGVQMFLSQVVIAIILGIKVTDHSDDLSKGIAILLVVMVCTFVSSFAWSWGPLGWLIPSETFPLETRSAGQSVTVCVNLLFTFVIAQAFLSMLCHFKFGIFLFFSGWVLVMSVFVLFLLPETKNVPIEEMTERVWKQHWFWKRFIDDAADEKVAHVSNGNGFDPTSRL

SEQ ID NO: 10 - protein, 522 aa, Artificial Sequence
  Lr67 allele based on SEQ ID NO. 3
MAVGGFTNAAGGADFEAKITPIVIISCIMAATGGLMFGYDVGVSGGVTSMPPFLKKFFPTVYRKTVEEKGLDSNYCKYDNQGLQLFTSSLYLAGLTSTFFASYTTRRLGRRLTMLIAGFFFIGGVVLNAAAQDLAMLIVGRILLRCGVGFANQAVPVFLSEIAPSRIRGALNILFQLNVTIGILFANLVNYGTNKIKGGWGWRLSLGLAGIPAVLLTLGALFVVDTPNSLIERGRLEEGKTVLKKIRGTDNIELEFQELVEASRVAKEVKHPFRNLLKRRNRPQLVISIALQIFQQFTGINAIMFYAPVLFNTLGFKNDASLYSAVITGAVNVLSTVVSIYSVDKLGRRMLLLEAGVQMFLSQVVIAIILGIKVTDHSDDLSKGIAILLVVMVCTFVSSFAWSWGPLGWLIPSETFPLETRSAGQSVTVCVNLLFTFVIAQAFLSMLCHFKFGIFLFFSGWVLVMSVFVLFLLPETKNVPIEEMTERVWKQHWFWKRFIDDAADEKVANVSNGNGFDPTSRL

SEQ ID NO: 11 - protein, 473 aa, Artificial Sequence
  Lr67 allele based on SEQ ID NO. 4
MPPFLKKFFPTVYRKTVEEKGLDSNYCKYDNQGLQLFTSSLYLAGLTSTFFASYTTRRLGRRLTMLIAGFFFIGGVVLNAAAQDLAMLIVGRILLRCGVGFANQAVPVFLSEIAPSRIRGALNILFQLNVTIGILFANLVNYGTNKIKGGWGWRLSLGLAGIPAVLLTLGALFVVDTPNSLIERGRLEEGKTVLKKIRGTDNIELEFQELVEASRVAKEVKHPFRNLLKRRNRPQLVISIALQIFQQFTGINAIMFYAPVLFNTLGFKNDASLYSAVITGAVNVLSTVVSIYSVDKLGRRMLLLEAGVQMFLSQVVIAIILGIKVTDHSDDLSKGIAILLVVMVCTFVSSFAWSWGPLGWLIPSETFPLETRSAGQSVTVCVNLLFTFVIAQAFLSMLCHFKFGIFLFFSGWVLVMSVFVLFLLPETKNVPIEEMTERVWKQHWFWKRFIDDAADEKVANVSNGNGFDPTSRL

SEQ ID NO: 12 - protein, 475 aa, Artificial Sequence
  Lr67 allele based on SEQ ID NO. 5
MAGGGEFEAKITPIVILSCMMAATGGLMFGYDIGVSGGVTSMPAFLKEFFPEVYRKTVEEEELDSNYCKYDNEKLQLFTSCLYLAGLIATFFASHITRRQGRRATMLISGFIFIAGVAFNAAAQNLAMLIIGRVLLRSGVGFANQAVPVFLSEIAPSQIRGALNILFQLNITLGILFSNLVNYATNKIKGGWGWRLSLGLGGLPALLLTLGAFMVVDTPNSLIERGHLEEGKVVLRKIRGIDNIEPEFLELLHASRVAKEVKHPFRNILKRKNRPQLVICIALQIFQQFTGINAIMFYAPVLFNTLGFKNDASLYSAVIIGAVNVVSTVVSIYSVDRLGRRILLLEAGVQMFLSQLVIAVIIGMKVKDHSEDLSKGYAVLLVVLVCIFVSAFAWSWGPLSWLIPSEIFPLETRSAGQSIAVCVNLLCTFVIAQAFLSMLCFFKFGIFLFFSGCVLLMSTFVLFLLPETKNVPLKR

SEQ ID NO: 13 - protein, 512 aa, Artificial Sequence
  Lr67 allele based on SEQ ID NO. 6
MAGGGFTTSGGEFEAKITPIVILSCMMAATGGLMFGYDIGVSGGVTSMPAFLKEFFPEVYRKTVEEEELDSNYCKYDNEKLQLFTSCLYLAGLMATFLASHITRRQGRRATMLISGFIFIAGVAFNAAAQNLAMLIIGRVLLRSGVGFANQAVPVFLSEIAPSRIRGALNILFQLNITLGILFSNLVNYATNKIKGGWGWRLSLGLGGLPALLLTLGAFLVVDTPNSLIERGHLEEGKSVLRKIRGIDNIEPEFLELLDASRVAKEVKHPFRNILKRKNRPQLVISIALQIFQQFTGINAIMFYAPVLFNTLGFKNDASLYSAVITGAVNVVSTVVSIYSVDRLGRKMLLLEAGAQMFLSQLVIAVIIGMKVKDHSEDLSKGFAVLLVVLVCIFVSAFAWSWGPLSWLIPSEIFPLETRSAGQSIAVCVNLLCTFVIAQAFLSMLCFFKFGIFLFFSGCVLIMSTFVLLLLPETKNVPIEEMTERVWKQHWLWNRFIDEDDCVKEEKVVTGN

SEQ ID NO: 14 - protein, 500 aa, Artificial Sequence
  Lr67 allele based on SEQ ID NO. 7
MAGGGFVSASGESHFEAKITFAVIISCIMAATGGLMFGYDIGISGGVTSMPSFLEKFFPEVYRKIQDHGVDSNYCKYDNQTLQLFTSSLYLAALVATMFASSVTRTLGRKQTMLIAGIFFIVGTVLNAVANSLLLLIVGRILLRCGVGFANQAVPVFISEIAPTRIRGALNIMFQLNITIGILIANIVNYFTAKIEGGYGWRISVALAGIPAIMLTFGSLLVHDTPNSLIERGLEDEGKAVLKKIRGVENVEPEFQEILKASKVAKAVKNPFQNLLKRHNRPPLIIAVMMQVFQQFTGINAIMFYAPVLFSTLGFKSDASLYSAVITGAVNVLSTLVSVYFVDKAGRRMLLLEACVQMFVSQMVIGTVLGLKVQDHSDSLNKGLGVLLVVMVCTFVASFAWSWGPLGWLIPSETFPLEARSAGQSVTVFTNMLFTFIIAQGFLSMMCHLKFGIFFFFSAWVLAMAIFTVLLIPETKNIPIEEMTDKVWRNHWFWKSYMED

SEQ ID NO: 15 - DNA, 20 bp, Artificial Sequence
  sense primer for SEQ ID NO. 2
atggccggcggaggtttcac

SEQ ID NO: 16 - DNA, 20 bp, Artificial Sequence
  sense primer for SEQ ID NO. 3
atggccgtcggaggtttcac

SEQ ID NO: 17 - DNA, 20 bp, Artificial Sequence
  sense primer for SEQ ID NO. 4
atgcccccgttcttgaagaa

SEQ ID NO: 18 - DNA, 20 bp, Artificial Sequence
  sense primer for SEQ ID NO. 5
atggctggcggcggcgagtt

SEQ ID NO: 19 - DNA, 22 bp, Artificial Sequence
  sense primer for SEQ ID NO. 6
atggctggcggaggcttcacaa

SEQ ID NO: 20 - DNA, 20 bp, Artificial Sequence
  sense primer for SEQ ID NO. 7
atggcgggaggaggatttgt

SEQ ID NO: 21 - DNA, 20 bp, Artificial Sequence
  antisense primer for SEQ ID NO. 2
ttacaacctggaggttggat

SEQ ID NO: 22 - DNA, 20 bp, Artificial Sequence
  antisense primer for SEQ ID NO. 3
ttacaacctggaggttggat

SEQ ID NO: 23 - DNA, 20 bp, Artificial Sequence
  antisense primer for SEQ ID NO. 4
ttacaacctggaggttggat

SEQ ID NO: 24 - DNA, 20 bp, Artificial Sequence
  antisense primer for SEQ ID NO. 5
tcatctcttcaatgggacat

SEQ ID NO: 25 - DNA, 20 bp, Artificial Sequence
  antisense primer for SEQ ID NO. 6
tcaattgccagtgactactt

SEQ ID NO: 26 - DNA, 20 bp, Artificial Sequence
  antisense primer for SEQ ID NO. 7
ttaatcttccatataactct

SEQ ID NO: 27 - DNA, 744 bp, Artificial Sequence
  Hybridization probe for SEQ ID NO. 2
ttcaccagcggcggcggcgccggagatttcgaggccaaaatcactcccatcgtcatcatttcatgtatcatggctgccaccggaggtctcatgttcggctacgacgtcggcgtttcaggtggtgtgacgtctatgcccccgttcttgaagaagttttttccgacggtatacagaaagacagtggaagagaaaggactggacagtaattactgcaaatacgacaaccaaggattacagcttttcacgtcgtctctgtatctggctggtttgacttcgacgttctttgcttcatacacgactcgtaggttgggaagaaggctgaccatgttgattgctggggtattcttcatctgtggagttgtccttaatgctgctgctcaagaccttgccatgctcatcgttggcagaatcttacttggctgtggtgttggctttgctaatcaggccgtgccagtgttcctctctgagatagcaccttcaagaatacgaggagcattgaatatattgttccagctcaatgtcacaattggcattctatttgctaaccttgtcaactatggcaccaacaaaatcaaaggtgggtggggttggaggctatctcttgggttggctggtatcccagcagttctgctaactttgggagccttgttcgtggtggacacccccaacagtttgattgaacgtggccgcttggaagaaggaaaaacagtgcttaaaaagattcgtggcactgacaacattgaa

SEQ ID NO: 28 - DNA, 774 bp, Artificial Sequence
  Hybridization probe for SEQ ID NO. 3
gtcggaggtttcaccaacgccgccggtggcgccgatttcgaggccaaaatcactcccatcgtcatcatttcatgtatcatggctgccaccggaggtctcatgtttggctacgacgtcggcgtttcaggtggtgtgacgtctatgcccccgttcttgaagaagttttttccgacggtatacagaaagacagtggaagagaaaggactggatagtaattactgcaaatacgacaaccaaggattacagcttttcacgtcgtctctgtatctggctggtttgacttcaacgttcttcgcttcgtacacgactcgtaggttgggaagaaggctgaccatgttgattgctgggttcttcttcatcggtggagttgtcctaaatgctgctgctcaagacctcgccatgctcatcgttggaagaatcttgcttggttgtggtgttggctttgctaatcaggccgtgccagtgttcctctctgagatcgcaccttcaagaatacgaggagcattgaatatattgttccagcttaatgtcaccattggcattctatttgctaaccttgtcaactacggcaccaacaaaatcaaaggtgggtggggttggaggctatctcttgggttggctggtatcccagcagttcttctgacattgggagccttgttcgtggtggacacccccaacagtctgatcgaacgtgggcgcttggaagaaggaaaaactgttcttaaaaagattcgcggcactgacaacatcgaacttgaattccaggaattggtt

SEQ ID NO: 29 - DNA, 633 bp, Artificial Sequence
  Hybridization probe for SEQ ID NO. 4
atgcccccgttcttgaagaagttttttccgacggtatacagaaagacagtggaagagaaaggactggatagtaattactgcaaatacgacaaccaaggattacagcttttcacgtcgtctctgtatctggctggtttgacttcaacgttcttcgcttcgtacacgactcgtaggttgggaagaaggctgaccatgttgattgctgggttcttcttcatcggtggagttgtcctaaatgctgctgctcaagacctcgccatgctcatcgttggaagaatcttgcttggttgtggtgttggctttgctaatcaggccgtgccagtgttcctctctgagatcgcaccttcaagaatacgaggagcattgaatatattgttccagcttaatgtcaccattggcattctatttgctaaccttgtcaactacggcaccaacaaaatcaaaggtgggtggggttggaggctatctcttgggttggctggtatcccagcagttcttctgacattgggagccttgttcgtggtggacacccccaacagtctgatcgaacgtgggcgcttggaagaaggaaaaactgttcttaaaaagattcgcggcactgacaacatcgaacttgaattccaggaattggtt

SEQ ID NO: 30 - DNA, 750 bp, Artificial Sequence
  Hybridization probe for SEQ ID NO. 5
ggcggcgagtttgaggcaaagataactccaattgtcatcctttcatgcatgatggctgctaccggaggtcttatgtttggatacgacattggcgtttctggtggggtgacatcgatgccagcattcctgaaagagttctttccggaagtgtacagaaagacagtggaagaggaagagttggacagcaactactgcaaatacgacaatgaaaagctgcaactattcacatcgtgtctgtaccttgcgggtttaatagccacattcttcgcgtcacatataactcggaggcagggaagaagagccaccatgttgatatcaggcttcattttcatcgctggagttgccttcaatgctgctgctcagaaccttgccatgctcatcattggtagagtcttacttggttctggagttggttttgctaaccaggccgtgccagtttttctttcagagatcgcaccttcacaaattcgtggagcattgaatatactgttccagctgaatatcacccttggcattctcttttccaaccttgtcaactatgccaccaacaaaatcaaaggtgggtggggttggaggctatctctgggtttgggtggtttaccagcattgcttcttacgttgggggctttcatggtggtggacaccccgaacagtctcatcgagcgtggccacttggaagaaggaaaagttgtgctgagaaagattcgtgggattgacaacattgaacccgagttcttggagcttcttcac

SEQ ID NO: 31 - DNA, 756 bp, Artificial Sequence
  Hybridization probe for SEQ ID NO. 6
acaagcggcggcgagtttgaggcaaaaataactccaattgtcatcctttcatgcatgatggctgctaccggaggtcttatgtttggatacgacattggcgtttctggtggtgtgacatccatgccagcattcctgaaagagttctttccggaggtgtatagaaagacagtggaagaggaagagttggacagcaactactgcaaatacgacaatgaaaagttgcaactattcacatcgtgtctgtaccttgcgggtttaatggccacattcttggcgtcgcatataactcggagacagggaagaagagccaccatgttgatatcgggattcatcttcattgctggagttgccttcaatgctgctgctcagaaccttgccatgctcatcattggtagagtcttacttggttctggagttggttttgctaaccaggccgtgccagtttttctttcagagatcgcaccttcaagaatacgtggagcattgaatatacttttccagttgaatatcacccttggcattctcttttccaaccttgtcaactatgccaccaacaaaatcaaaggtggatggggttggaggctatctctgggtttgggcggtttaccggcactacttctcacgttgggggctttcttggtggtggacaccccaaacagtctcatcgagcgtggtcacttggaagaaggaaaatctgtgctgagaaagattcgtggcattgacaacattgaacctgagttcttggagcttcttgac

SEQ ID NO: 32 - DNA, 762 bp, Artificial Sequence
  Hybridization probe for SEQ ID NO. 7
gtatcagcttcaggggaatcccactttgaggcaaaaatcacgtttgccgtcataatatcttgcataatggctgccaccggtggccttatgtttggttatgatattggaatttcaggtggtgtcacatcaatgcctagctttctagaaaagtttttccctgaggtatacagaaagatacaagatcatggtgtggacagtaactactgcaaatatgacaatcaaactctacaattgttcacatcctctctgtacctcgcagctttggtagccaccatgtttgcctcttccgtcaccaggacgctaggccggaagcaaactatgttgattgctggcatttttttcatcgttggaactgtcttgaatgctgtagcaaatagcttgctactgctaattgttggcagaatcttacttggttgtggtgttggtttcgccaatcaggctgtgccagtgtttatttctgagattgcacccacaagaattcgtggagcacttaacatcatgttccagcttaatatcaccattggaattctcatagcaaacattgtcaactacttcacagcaaagattgaaggtggatatgggtggaggatatcagtggctttggcagggattccagcaataatgctgacatttgggtccctcctggtgcatgacactcccaacagtcttattgagaggggtttggaggatgaagggaaggctgtgctaaagaagattcgtggtgtggaaaatgttgagccagagtttcaggagattcttaaa

SEQ ID NO: 33 - DNA, 5258 bp, Glycine max
  nucleic acid for wild type Lr67 gene SEQ ID NO. 2 (Glyma.0 1G238800.1.Wm82.a2.v1)
atggccggcggaggtttcaccagcggcggcggcgccggagatttcgaggccaaaatcactcccatcgtcatcatttcatgtatcatggctgccaccggaggtctcatgttcggctacgacgtcggcgtttcaggtaatcatcttttcttcttctcctctctctctttctttggaacagtggaagtagcttggcacttgtgttgtgaaaaagcaattaattttgggtcggttaatcttatcttaggtggataatgaattactgttttttctaactaaaatcgattctgatttgccttgacgaggcgctattatgggaatcccacgtaaatttccaaaattgctcaaccaccatacttaaccaaacaggttataagggcatttttgagatttcacctaactagttcgctaatgccaatctgtcgtcctgtcacttttgccaaaacctgttacagtactttaatatgtgcagtaaaatgttaaccatattaattgaggaaaattatacctcaatactgactagtaagatttatattaatatgatgtattaggtggtgtgacgtctatgcccccgttcttgaagaagttttttccgacggtatacagaaagacagtggaagagaaaggactggacagtaattactgcaaatacgacaaccaaggattacagcttttcacgtcgtctctgtatctggctggtttgacttcgacgttctttgcttcatacacgactcgtaggttgggaagaaggctgaccatgttgattgctggggtattcttcatctgtggagttgtccttaatgctgctgctcaagaccttgccatgctcatcgttggcagaatcttacttggctgtggtgttggctttgctaatcaggttcatcaatcatcatattttatttttcaaaactcaattgcacaatgttaattaattggatttaaatgaattcgtgtatattgctagtaagtattaattataaataaataaataaatattgcacatgtatcatttatgtgtgcgtgattcatccattgattaatatatatatatatatatatatatatatatatataatactttgaaattatttatggatagaacccattttgtttcaataattaattttagtttgtgttttgttgtgaaaaaaaaatatttttttctctcacagctaaagaaaaaaaatttaaaattatctatagatctcacatctctttctctcactttaattcaattttttttcattttttttctcttccacataatcaaactcatccttgatgtgtttatttaagttttttttaagaattagaataaaaaaattactactatttttattagtttgagaagttcttttgagtagctttttgaaaaattataattataaccaattatgaatgtaacgaaaaaaataagttctatttatttttataaaacccgtgaaaagtaaataactttttttatacttctttgaaattatgttttataaaaacataaaccaaaggactctcgaattagcttccactttacacaaaagctaaaaaatgcaagttactttttctttttttatacaatcttaagatcaccttttaaaaaaattagcttaatcttttgaaccctctgtctattacctatcacaggtgtcaccgcgtctatgattgttcacatttgttcaacaacatcacagttcgattatttgctcaataggatatggttttgttggttcagagagtttaaatgaagatattgttacttgcatagtagatatctataatacaccttattaatccaatttgaagtatcatcggtcatgttttactcctaaaaacataattagtcaagattccttttttctttgggtccaatgacgaaaattagtatattcagtttttattttgtgtgtactgcgtgtgtctatagagagaacatggccgacgtatgaatatatacgttaaatatatttttttatcaaaaaaaatacccaaaggctttgtcgtggaggagactgatgaatgtccccacaacaatattgtggactgtcatgcgccaacagaaggcaaatctgtagtttaaactgagacttttgtgacaattgtccataatttttccttgtactcccaaagtcctaatcacccattaattcctccaatcgagttcttccaccttttatacttataaatattcttactattggatgtttgtgacacttgttaatagggaccactttttgtaccatattttatgatttttagttttgtctctttgacacaattctttgaagattcctattctgaaatgagtttccatattttaaattaaaaagtcctaggcctttttgctatagagagaaaaaaaaagcattctgcagtttaattagtatttttgtagctcacaaattctatttttctgtcgtctttttacttttcagtcattacttggactaattttgttgattcctcacttatacaagcaatacaactaagttgtttgttgtccaaatcaattaataatagtggatttaaaataacctcttactaatgatcaatggtatcagaaagcaaactaacattactgataatagttgcggtaaaaaataaagacttctttggttgggtatagggtatgcatatctcaatcatctttcttgtttcaatttgatgacaggccgtgccagtgttcctctctgagatagcaccttcaagaatacgaggagcattgaatatattgttccagctcaatgtcacaattggcattctatttgctaaccttgtcaactatggcaccaacaagtaagtactattttatgtgcttccccatagcttacttttcttcaactgagttacttcattggaattcgcaaataaaacgaatcagacgatgtatgtgttagaagagtaagttattgtcaaatagtagtcccaccacactttttttttcttcttcctgtgtcgtgctcaggatttcgtcacaagtaataacgtgggtggtaccggtcaaaattgttaacctgttgaatgggaaacaacaaaattataatctttagtgcgactttttggttttatggccgtaattagaactcgaaagtgtagcacatgctggcttctttgtagtgtgctatcagctagcgggacaagtgtcaatttagtgtttagccagcaagcatctgttttgggtcattagtcaaaggtgcatgcttcagagaaaaatggataagaatcctacgcaattttaaactgcggaacatttcagacgttgaacgtaatactttttgtgatacaatatactgtaacatgttttcaacagtggcgtcttgaaccgcaccccttgtagttttttcttgatttgcacccagattttgcacttaattttcttaaaagagagagaaccccgagtcctgactttcattttcatgtgtcgtggactcgtggtaacccatcttggcaaaggccaacatggtccatcccaaatattttacgcatttaatagttgatgaaaaatgatgtagtagtacgagtaatattacactcacaatttttttattaaacaataatacataaacattttatcaatatctctttttttatttatctctttctattatactataaatcttataatatctatattcttctctttcttgttctctctaggtgtattgtagtataataggtgttcaccaaacattttatttttataattctttttatgttgtgaaaatctatgttgagattaatgtttctctaagaaaaacaccctgctgtaaaaaagaaactagcgtatcctattggccggaaactcctcgctaagtaaaggtatgagagagggttattatacgcaaccatacccttacggaaataaagagactgttttcggatttgaatcataatcaatcagtatgaggcacaactttaccgttgtggcaggtctcacaatcaaaacactctgctgtagaatgtaaaatttgtaaatgaataaaagaaatgccagatagctagtacgtacttatgaaatcacccaagtctaaacatcatggctattccggagattttggtgaagaaatcaatcttgctgaatcattatgttttcgcgtaaaatgagacataataattgaattgtgaaatttctgtttttctgtcttgatttaccttgtactgctgctgattgatgttgtgttgtgacctgcaacaacagaatcaaaggtgggtggggttggaggctatctcttgggttggctggtatcccagcagttctgctaactttgggagccttgttcgtggtggacacccccaacagtttgattgaacgtggccgcttggaagaaggaaaaacagtgcttaaaaagattcgtggcactgacaacattgaacttgagttccaggaattgcttgaggccagtcgtgtggctaaagaggtcaagcatccattcaggaatcttctcaagcgcaggaaccgaccccaacttgtcatttccgttgccctacaggtatttattcaagtgttgactttaaataactataatatgcactatcattgatctgatcttaataatgtttgtgttgtaaaacagatattccagcaattcacaggcatcaacgcaatcatgttttatgccccggtgctgttcaacacgttaggattcaagaacgatgcatccctgtattctgctgtgataactggggctgtgaacgtgctctccactgttgtatcgatctactcagtggacaaagtgggacgtcgcattctgttgctggaagctggtgtgcagatgttcttgtctcaagtggtgattgcaatcatactgggaataaaggtgacggaccattctgatgacctttcaaaaggcattgcaattctggtggttgtgatggtctgcactttcgtgtcttcctttgcatggtcttggggtcctcttgggtggcttatccccagtgagactttcccattggagactcgttccgctggtcagagtgtcaccgtttgtgttaacctgctcttcaccttcgtcattgcacaagccttcctctctatgctctgccacttcaagtttggtatcttcttgttcttctctggctgggtcctcgtcatgtctgtcttcgtgcttttcctgttgcccgagactaagaatgtccccatcgaagagatgaccgagagagtgtggaagcagcattggttctggaagaggtttatcgatgatgccgcggatgagaaagtagcccatgtctctaatggtaatggatttgatccaacctccaggttgtaa

SEQ ID NO: 34 - DNA, 4132 bp, Glycine max
  nucleic acid for wild type Lr67 gene SEQ ID NO. 3 (Glyma.1 1G004800.1.Wm82.a2.v1)
atggccgtcggaggtttcaccaacgccgccggtggcgccgatttcgaggccaaaatcactcccatcgtcatcatttcatgtatcatggctgccaccggaggtctcatgtttggctacgacgtcggcgtttcaggtaatcatcttttcttctctctctctctctatatatatatgtgtgttctttccttcttttacaattcattaaccttatttgtgttctttttcacgatctcaaacacatatatatgagtatatgacatacctagctagacactgttttttttttttttttttacaaagagaaaacaagaatcgttttaaatattttttccctttatgaacaaaacaataagaaacaataccatccatattttcaagagactccatccatctgttttttttttcttctttttttccgattatatgtaaaagtaattacaaggaagaaaagtggaattaacttagcagttgtgctgtgaaaaatcaattaattttaggtcggataatcttatcttattaggtggataatgaaatactacttttttattttattttattctacctaaaaaagaggctcaatcgattctgattttccttgacgaggatcacgattcatgtggcgctaatatgggaatcccacgtaagttcccaaaattgctcaaccaccatacatacttaaccaaacaacctgtaagggcattttcgagatttcacttaacagctcgttaatgccaatgagtcgtcctgtgaattttgccaaaaccagttacagtaacagctcgctaactttaatatgcacagtaaactatgattattatgtgcattaaatattaattaatatgatgtaataggtggtgtgacgtctatgcccccgttcttgaagaagttttttccgacggtatacagaaagacagtggaagagaaaggactggatagtaattactgcaaatacgacaaccaaggattacagcttttcacgtcgtctctgtatctggctggtttgacttcaacgttcttcgcttcgtacacgactcgtaggttgggaagaaggctgaccatgttgattgctgggttcttcttcatcggtggagttgtcctaaatgctgctgctcaagacctcgccatgctcatcgttggaagaatcttgcttggttgtggtgttggctttgctaatcaggttcgcttcatcatcatttatttttcactttattaaattaattggagttaaatgaatccgtgttgacttattgatcagttattatttgctaatagggtagtagtaattaaaaatacatattaattgcacatataacatgtactttgtatatagaagatttacgctttgagattatttatggatagaaactattttgtttcagttttatttttatacttttaaggtagtcatttaagttcttctaacgaaccagaataagaaaaatcatatttttaattattttaggaagttctttttatgagcttaaaaaaattataattataaccaaattgactcacgaattaaactctttttttttatacttatttaaaattatgttttataaaatcataaacaaaattgactcacgaattagcttccagtttactcacaatcactaacaagctagcaaattaatattttttatacaactttgaaaatcacttgtttttataattagcttaatcaattgaaccttctgtccattatctatcacagttgtcaccgtgtctaggattgttcacgtgtgttcaacaacagttcgattatttgctcaataggatatggttttgttgtttcgagagtttaaaaagaagatactgttacatgcatatatagtacatatctataatacactttattaatccaatttgaagtatcatcggtcttgttttactcctcaaaacataattagtcaagattcctttgtgcctttcgtctaaagacgaaaattagtatattcattttttattttgtgtgtctatattatatagagagaacacggccgacgtatgaatatatacgttaaatatatttaacattttttaaaaaaaaaacaaaaggctttgtcgtggaggagacagatgaatgtccccacaacaatattgtggaatgtcatgcgccaacggaaggcaagtctgtagtttaaactgagacttaattttgtgacaattgtccttaatttttccttgtactcccaaagtcgtaatcacccattaatttctccaatcgagttccaccacctttaatactttaaaatattcttactattggatgtttgtgacacttgttaatagggaccactttttgtaccatattttatgattattattagttttgtctctttgagacaattctttgaagctacctattttgaaatgagtttccatattagaattaaaaagtcctaggcctttttgccatagacaaaaagaaaaaacgcattctgcaatttagtatttttgtatctttcaaattctatttttctgccacttttttactcttcagtcattacttgggttaactttgttgattcctcacttatacaagcaatacaactaagctgtttgttgtccaatttgctaacaattacttgttgactcctcactaatagtggaatagttgatttaaaataacctcttactaataattaatggtatcagatagaaaattaatattactgataatagttgctgttaaaaaaaaaagacatttttggttgggtatccatatctgaatcatctttgttgtttcaatttgatgacaggccgtgccagtgttcctctctgagatcgcaccttcaagaatacgaggagcattgaatatattgttccagcttaatgtcaccattggcattctatttgctaaccttgtcaactacggcaccaacaagtaagtactattttatgtgctcccccattgcttacttttcttcaactgagttacttcattgaaagttgccaataaaacgaatcagatgatatgtgtgttagaaaagagtaagttatagtcaaaatagaacagagagaatggtaacccaaagcccgcatggtccagcccaaaatgtatgacggatttaatagttgatacaaaacgatgtagtagactggtagtactagtaatattacactgacattttttcgtaacaacagaatcaaaggtgggtggggttggaggctatctcttgggttggctggtatcccagcagttcttctgacattgggagccttgttcgtggtggacacccccaacagtctgatcgaacgtgggcgcttggaagaaggaaaaactgttcttaaaaagattcgcggcactgacaacatcgaacttgaattccaggaattggttgaggccagtcgtgtggctaaagaggtgaagcatccattcaggaatcttctcaagcgcaggaaccgaccccaactcgtcatttccattgcccttcagatattccagcaattcacaggcatcaacgcaatcatgttctatgccccggttctcttcaacaccttaggattcaagaacgatgcatccctctattctgctgtcataactggtgctgtgaatgttctctccaccgttgtatccatctactcggtggacaaactgggacgtcgcatgctcctgctggaggctggtgtgcagatgttcttgtctcaagtggtgattgcaatcattctgggaataaaggtgacggaccattctgatgacctttccaaaggcattgcaattctggtggttgtgatggtgtgcactttcgtgtcttcctttgcatggtcttggggtccccttgggtggcttatcccgagtgagactttcccattggagactcgttccgctggtcagagtgtcaccgtttgtgtcaacttgctcttcaccttcgtcattgcacaggcattcctttctatgctctgccacttcaagtttggcatcttcttgttcttctctggctgggtcctcgtcatgtctgtcttcgtgcttttcctgttgccagaaacaaagaatgtccccattgaagagatgaccgagagagtgtggaagcagcattggttctggaagaggtttatcgatgatgccgcagatgagaaagtagccaatgtctctaatggtaatggatttgatccaacctccaggttgtaa

SEQ ID NO: 35 - DNA, 3267 bp, Glycine max
  nucleic acid for wild type Lr67 gene SEQ ID NO. 4 (Glyma.1 1G004800.2.Wm82.a2.v1)
atgcccccgttcttgaagaagttttttccgacggtatacagaaagacagtggaagagaaaggactggatagtaattactgcaaatacgacaaccaaggattacagcttttcacgtcgtctctgtatctggctggtttgacttcaacgttcttcgcttcgtacacgactcgtaggttgggaagaaggctgaccatgttgattgctgggttcttcttcatcggtggagttgtcctaaatgctgctgctcaagacctcgccatgctcatcgttggaagaatcttgcttggttgtggtgttggctttgctaatcaggttcgcttcatcatcatttatttttcactttattaaattaattggagttaaatgaatccgtgttgacttattgatcagttattatttgctaatagggtagtagtaattaaaaatacatattaattgcacatataacatgtactttgtatatagaagatttacgctttgagattatttatggatagaaactattttgtttcagttttatttttatacttttaaggtagtcatttaagttcttctaacgaaccagaataagaaaaatcatatttttaattattttaggaagttctttttatgagcttaaaaaaattataattataaccaaattgactcacgaattaaactctttttttttatacttatttaaaattatgttttataaaatcataaacaaaattgactcacgaattagcttccagtttactcacaatcactaacaagctagcaaattaatattttttatacaactttgaaaatcacttgtttttataattagcttaatcaattgaaccttctgtccattatctatcacagttgtcaccgtgtctaggattgttcacgtgtgttcaacaacagttcgattatttgctcaataggatatggttttgttgtttcgagagtttaaaaagaagatactgttacatgcatatatagtacatatctataatacactttattaatccaatttgaagtatcatcggtcttgttttactcctcaaaacataattagtcaagattcctttgtgcctttcgtctaaagacgaaaattagtatattcattttttattttgtgtgtctatattatatagagagaacacggccgacgtatgaatatatacgttaaatatatttaacattttttaaaaaaaaaacaaaaggctttgtcgtggaggagacagatgaatgtccccacaacaatattgtggaatgtcatgcgccaacggaaggcaagtctgtagtttaaactgagacttaattttgtgacaattgtccttaatttttccttgtactcccaaagtcgtaatcacccattaatttctccaatcgagttccaccacctttaatactttaaaatattcttactattggatgtttgtgacacttgttaatagggaccactttttgtaccatattttatgattattattagttttgtctctttgagacaattctttgaagctacctattttgaaatgagtttccatattagaattaaaaagtcctaggcctttttgccatagacaaaaagaaaaaacgcattctgcaatttagtatttttgtatctttcaaattctatttttctgccacttttttactcttcagtcattacttgggttaactttgttgattcctcacttatacaagcaatacaactaagctgtttgttgtccaatttgctaacaattacttgttgactcctcactaatagtggaatagttgatttaaaataacctcttactaataattaatggtatcagatagaaaattaatattactgataatagttgctgttaaaaaaaaaagacatttttggttgggtatccatatctgaatcatctttgttgtttcaatttgatgacaggccgtgccagtgttcctctctgagatcgcaccttcaagaatacgaggagcattgaatatattgttccagcttaatgtcaccattggcattctatttgctaaccttgtcaactacggcaccaacaagtaagtactattttatgtgctcccccattgcttacttttcttcaactgagttacttcattgaaagttgccaataaaacgaatcagatgatatgtgtgttagaaaagagtaagttatagtcaaaatagaacagagagaatggtaacccaaagcccgcatggtccagcccaaaatgtatgacggatttaatagttgatacaaaacgatgtagtagactggtagtactagtaatattacactgacattttttcgtaacaacagaatcaaaggtgggtggggttggaggctatctcttgggttggctggtatcccagcagttcttctgacattgggagccttgttcgtggtggacacccccaacagtctgatcgaacgtgggcgcttggaagaaggaaaaactgttcttaaaaagattcgcggcactgacaacatcgaacttgaattccaggaattggttgaggccagtcgtgtggctaaagaggtgaagcatccattcaggaatcttctcaagcgcaggaaccgaccccaactcgtcatttccattgcccttcagatattccagcaattcacaggcatcaacgcaatcatgttctatgccccggttctcttcaacaccttaggattcaagaacgatgcatccctctattctgctgtcataactggtgctgtgaatgttctctccaccgttgtatccatctactcggtggacaaactgggacgtcgcatgctcctgctggaggctggtgtgcagatgttcttgtctcaagtggtgattgcaatcattctgggaataaaggtgacggaccattctgatgacctttccaaaggcattgcaattctggtggttgtgatggtgtgcactttcgtgtcttcctttgcatggtcttggggtccccttgggtggcttatcccgagtgagactttcccattggagactcgttccgctggtcagagtgtcaccgtttgtgtcaacttgctcttcaccttcgtcattgcacaggcattcctttctatgctctgccacttcaagtttggcatcttcttgttcttctctggctgggtcctcgtcatgtctgtcttcgtgcttttcctgttgccagaaacaaagaatgtccccattgaagagatgaccgagagagtgtggaagcagcattggttctggaagaggtttatcgatgatgccgcagatgagaaagtagccaatgtctctaatggtaatggatttgatccaacctccaggttgtaa

SEQ ID NO: 36 - DNA, 7493 bp, Glycine max
  nucleic acid for wild type Lr67 gene SEQ ID NO. 5 (Glyma.1 0G249500.1.Wm82.a2.v1)
atggctggcggcggcgagtttgaggcaaagataactccaattgtcatcctttcatgcatgatggctgctaccggaggtcttatgtttggatacgacattggcgtttctggtaatgttttcatgcttcaattccctctcttatttgaaccatctgttcctaaaataaaaactttaatttgcatgttttctttaactcaacatgatctagctagatccaaaagaatttatcttttcgatcttattatgggactaattttcccgtagtatttgactataagtagctttttaatttattttattttatttttatcaataaatgttaattattaattactttattaaaatttgtgactttttcttcttttttaagtcacttgaccaaccttatatttttcttataactttttggctaatatttattttttatccatttaaaagaataaaacttatggaagaaaaaatgaaagaaattacagaaaacttgaccaggaaactatttttaattaaaaaagaaaagaaaacatgcatacaattacatcacaccacatgcattcgtcaaatgactcatacagtcaaatgactacaaatcaaccggaattggatttattagcataaggccataagggattaggcactataatatgatgaatcaaagtttacattttttgtcaagagatatgttgatattaagtgcttaatcatgtcttaaaagtaaattgctttcaaagaaaaatacgagagacatataaataaaagactgcaaatcagaagaatatgacatctcataggtgtttctattttttgttacttaaaacacatgttttgaaaataaatttcataatttaattattttagatgagaagttagttgttttaattttctggtacgtgagttagtgacatgaaattattttagaaaataaattagaaactaaaaaggcaaaaaagaaatcaaacagaccattatattctcagatgttttttattttatttttatagtttaatttacggaaaataaatcttcatattcgattttatttttttactggaaaagtcttccattgtacttgatcacatctttttatgacgatagctcatgaaaagttgagtttcatatatccctctagaacccgaagcaagaacccaatttttcataaacaacaaaatgttagtttttcacaaaaagagattcataaattattatgaaactgatatatttatatttaaaatcaactttctttaattttcacccctttaaccagcagtttctagtctcaagtgcaattgattttatttatggggagatttgaatcaggtggggtgacatcgatgccagcattcctgaaagagttctttccggaagtgtacagaaagacagtggaagaggaagagttggacagcaactactgcaaatacgacaatgaaaagctgcaactattcacatcgtgtctgtaccttgcgggtttaatagccacattcttcgcgtcacatataactcggaggcagggaagaagagccaccatgttgatatcaggcttcattttcatcgctggagttgccttcaatgctgctgctcagaaccttgccatgctcatcattggtagagtcttacttggttctggagttggttttgctaaccaggttattcttttttctccccctctctcattttttttaagagaaaaaatgattgtatatttattagactcatccaactataatttatcatatataataattttgttaatttttaaataattattttaaaataattcgaatgataatttattattaaattattttatattttcagtgtataaatattaactctttttttgtcacaatatattacccttccgaattcatatatatatatatatatatatatatatatatatatatatataagtataagaaggagatcctttgtttaagaataaatctaaagagttactcttaaatccatccgagtcatttattcactaaacatacattagatgatgacaattctccttctcccctttgcggaaaacccaataactcgtttagtcttcctctttccattttggcattttctctgaagcgctttttctttctttttacgatgctctatggtactctatattattatgtggggagatgcaagctagatttctatgtaagtgaccacaccaatcaagtatatacaaaatttctaatttgacatcagtataggccctataggggaagtgcaacacacaatttgatatcagtatagggaagtgcagcacgcattatattataaaattctcatgttataaattattttataaaaatagaaataaacagagtgtgtcagaaacatataatattacgggtaactacaaaatatgggtatagaacgtttttttagaacagatcatgaacaaggccgacaatggatgacaacaaataacccttatacacataagattgtacgtacttaattagcattcataaacaaaacgaccaagaccatataacactagtaatatactatgattttattttgtacgtacttcagcatatctttaaatgatacgatggtaacttgtagtgttcaattattatgtgacatttatacataattttattattgcgtttttctaatattatgatttactaaattaaaggagaaaataaattaataagctgtgattaattgactaaatttaaagaaaactatttgtaaatactagtttaaatgaaaatagagattcattaaatcacgtgacacgttaaaaaaataaatcaagagaagagtttattttcaagagtgggtatttaaatgtaatatttaaaataataattcatataaattataaaaatatatatttttaccttaataaaatagttttaaaacatgattttaaaaaataatttgggagtgctgagttctacccttactctcttcaatccgtccttaaatacccataatcctcttcttgtaatctttatcttttattccttatttatatatatctcaattagccatttaagaaaacaataaatacgtaagtatagctctagacctctagtaataaaagcattaaaactaaatatttttttataaataattgtactaataaactataaaattataagttttgtattaaaaaaattaagagttaacatatgcaactttttggttaaaaaaaaaagttttgaggacaaaaaagaccggaatcacgcccgttccgacgggatctatcccattgatacatcttaatatgcctcgtctaggataatttcgtatttgctctacgaatgtcttttttgtaatttcccaaacagaatcaaggttttattttcctgaatcctgcggacattagaatcatatactaatacaacgtctttatcgtatgcctgtatgtttgagttttttttttttttttttctgacctatatgttttagttgatgtttgtaaaattgattgagtataattaattatataaaattaaagtaaaattcatatttatagacaaaaaactaactcgtaaaagtaactcttagttgtttcagcaaatacgtttgttggaagttgtatgacgccaaaccaatactaaaaaagcatgatatcttacaaaattaaagtccaatacacataaaagaagtataagttccaaattattactgcaaggtgagactcgtcaaagaggcttgaacatatatataattaaacacacgtcttcaaaattattttaatttaaaaataattctagaccaaaaaatcaattttaaatgttttgatccgtaaaaattaaacacaactcctgataactcatttatttcatcaattgaattagatttttgataaacttttgaatatgaaattaaataagtaaacatttacttaaagttatattatcttttaacataaaagtatcattgttatttaatgagtaaaaacttattaagctgacggtgagatattgacatattatgtcccttagtccaagacgaaaaataggttttgtagtttttacaagtaggtgtggtgtgtatatattaattacagagggagaacgagctggcatatttgaccaacctaaaggattaaaatataattataaagaaaatgataaatataaaaaatgtagaaagggcattgtcatgtagcgacaatataatgaatgaccatgcttcagtggaagggaagtttataattaaaaccaatagttttattggtaattgtacaaaagcttccttggcatgcactcccaaagtcgtaatcgtctttcttcaaacttcaacccaccttttatgctttaaattgttcttactatttgacgtttacgacacttgatggagatcactttgtattttctctcctttaattatataaagtcatttatattttatatatggctaagaatctcttttttgacattgaaagtgcaaacaaaacttcaacttgtgctactcttttttgtagtttacaaattatatctacatcttatccatataaacgataaagctaagccgataatggaaaataaattatttgactaatattttaatggtatgcgttggccttttggaacccactcatcttggttgctttaattatttttttttatgacaggccgtgccagtttttctttcagagatcgcaccttcacaaattcgtggagcattgaatatactgttccagctgaatatcacccttggcattctcttttccaaccttgtcaactatgccaccaacaagtaagtttcttgcgtgcatgctcccgatttcatctttttttttgtttgaataaatcatgagtgtgttaaaaacttgaaaatgtatagaaagaaacaaatcctagccattagaagcttaaccaatctccccagtgttattactcatgcccaagtgaaagcatcacttagacgtagtcagcaaattgaattaatggttatggtgagagtggtgctagtcaaaattttgaaaattgatggccaatattggagttgtcctccaaagagattctgctgtatagaaaaccaacaaaattatgaccatttctgttactcctcttcctctctttcacattcacatacccaaaagtcaagcacatgcttgcctactctgctaagtgttttgggcctccataaattgttcaaaaaccagaagcaactcaagcaagtatctgctaacaggacaagtgtccacctctaagaaacgggccacaattattttatttttcattttttattgggaaacctcaaattttcattttcacggtttcatcttcatcaattgcttccttccatgagtcttggtttggtaaccaacccatcttggtaaaggcccgcatggtctaggcccagaaatgttaacttgcttaatggttggtttgtgtgaaaatttgaactgactaaaactattgtatacatcactgtttcaaaataataataataataatataaacatccatccctcttcatgatcgaatattcacaaagattaataatatattttcctacaaatacttcaattattctttttaatattttatgaagaaaatattaaataaaataatattatttaagaaacattcaatactttactagtataaagatttgtagcaggatttagaatcatccaggttaacaaggtttcaattatttaatgccagtcaactgatatagacgtagcatcatttttacgtgactcaaaatgtaatgaaatccagtaatctagaattgaacaattaaaaaagccaggtaatagattctcgatcatgcaaaatcattgccaataaaaaatcgaattctaaagcatatgtcatttgcccatttgtcttgttttgattataccttgggtacttgttgattgttggtgatgctattgctgactattgttgatcgagcaatgacagaatcaaaggtgggtggggttggaggctatctctgggtttgggtggtttaccagcattgcttcttacgttgggggctttcatggtggtggacaccccgaacagtctcatcgagcgtggccacttggaagaaggaaaagttgtgctgagaaagattcgtgggattgacaacattgaacccgagttcttggagcttcttcacgcaagccgtgtggctaaagaagtcaaacaccctttcaggaatatcctcaagcgcaagaaccgaccccagctagtcatttgcattgccctgcaggtcagtgctatgttctcaaacaatttcctgtgagttaagtatgcaatttatctggtctcaacttgcattcttttggtccctacaaatttacatatatataatttagtctactcatctatcaacttcattatttttttttattaactctatgtctgtgtttagttttttcacatttacctattccaatgtgaccaaaagaatatgtataaatggaagacaattcttacatgttaacttctaaattttaggacttgagatgagtcaaaatccaaattttaatacaaataagagactattttaatagttcttgttggatagatcatacaacttttattgtgtcgttatcgaatcatccaaatattttttataatgttcaatgtgagagaggtatattagagattgtaatacgacgaaaataacattataaacaaatgacttctcaatgggtgaccaatcataaccttactaattgaatttgttgtgaaattgaaattttagagattaaggggatgtattcaattttattttatttttatggaaaaattctataaacacaaactttctaggaccaaattaacataagttgccacttttgagtataaaattgtgttttcagttgttcactcttttcctaacataattgaaacaatatgttattcttagatggctgagtatgctcaatcggaatttacttattgatttaattcttattccatttgggtgtcttcggaaaacagatttttcaacagttcactggcatcaatgccataatgttttacgccccagttctgttcaacacattgggattcaagaacgatgcttctctctactctgccgtgattattggtgctgttaatgtcgtctctactgttgtctcaatctattcagtagacagactcgggaggagaatactcttattggaagctggagttcaaatgttcttatctcaacttgtgatagcagtcataataggaatgaaggtgaaggaccattctgaggatctctcaaagggttatgcagtgctggtggttgtgttggtatgcatctttgtgtctgcatttgcatggtcatgggggcctcttagttggctgatcccaagtgagatattccctctggagactcgctcagccgggcaaagtatcgcggtgtgtgtcaacttgctctgcacatttgtcattgcgcaggcctttctctcaatgctgtgtttcttcaagtttggcatcttcttgttcttctctggatgtgtcttgctcatgtccacctttgtgcttttccttctccccgagacaaagaatgtcccattgaagagatga

SEQ ID NO: 37 - DNA, 7128 bp, Glycine max
  nucleic acid for wild type Lr67 gene SEQ ID NO. 6 (Glyma.2 0G144300.1.Wm82.a2.v1)
atggctggcggaggcttcacaacaagcggcggcgagtttgaggcaaaaataactccaattgtcatcctttcatgcatgatggctgctaccggaggtcttatgtttggatacgacattggcgtttctggtaattaatgctctcatgattcaattccccctcttgtttcaaccatctgttcctaaaataaaaactttaattttcttgtagtgtttgaggtataagtagctttttaatatttattttaaagaatactaagaattttttttttaacatattcctttaaatacttctttttaattgattaatatttattaaaaacttctttttcactcctttaaccacctctatgtttagtgtcttagtcactccatgaaactttctaactctattttcaagtgtagttgaagcgttgcatatgtactgttatattaaatccatgacccaattttcgtttttattgtagcttcaattatagtttcttctcttttaataaaaaaaatactgctttttttcttccgaaaataaaaatataaaaatataatttgatattatttctggcgatttgaatcaggtggtgtgacatccatgccagcattcctgaaagagttctttccggaggtgtatagaaagacagtggaagaggaagagttggacagcaactactgcaaatacgacaatgaaaagttgcaactattcacatcgtgtctgtaccttgcgggtttaatggccacattcttggcgtcgcatataactcggagacagggaagaagagccaccatgttgatatcgggattcatcttcattgctggagttgccttcaatgctgctgctcagaaccttgccatgctcatcattggtagagtcttacttggttctggagttggttttgctaaccaggttattcttttctctccccctctcccatttttatttatcacaatatataatatccttcagaattaatatatatatatatatatatatatatatatatatatgatctccatcgtactgaaataattttccttcaagatacagacaaatgaccttttaatttataccttaaagattgtacttaactagaatctatgaacgaccaacaattttgaataccagtaatatatgatttttttttttggtgacccgtgtattccttagatgagagtcaatcattaatctcttatgataaatatttttatatatatatgaccaaagagaattgaactcttaactatatatttaaaatgataatattatctatagacataattttaatatgtacttcagctcatctttaaaacatatgtcgcttatacaatggtaacgtgtagtgttcaattattatgtgacatttaacacatgcttttacataatgattttattatttcatttttttaatattagtttatctaaactaatagataaaataaattaattaataagactaaccaaatttaaagaaaatcatttttaaaaataagattaaaaaatagagattcattaaatgagacgagtttcgtgacacattaaaatgaataatttaaaagaaaaatttatatttaagagtggtatttaaatgtaatatttaaaataattaataaattataaaaatatgtatttttaccttaataaaataattttaatacatgattttaaaaaataatttaggagtgctgagttccacccttgttctcttcaaagcgtccttatataaaataaatacgtaaggatagcactagacctctagtaataaaagcattaaaagtaaatatttttttatttataaataatttatttttttaagaaataaatatttgtattagtaaaatataaaattataactattgtataaaataaaaaaactagagtcaacacacgtaatttttcctttaaaaaggacaaaaggccggaatcacattgattctaacagaatttatccccttaacaaatcataatacgtcacttctaagataattcatatttaagagactgtgttaagaagtgtgataatagcattactctttacctattacttgtgttggaagttgtttgacgccaaatcaatactaaaaatgcatgttattttacaaaattaaagtccaatacacataaattaaagaagtaggagctacaaagtgagacgcgtcaaagaggtttgaacgtatgtatatatatatatatatatatatatatatatggaatcaaataagtgaatatttacttaaaaatcagttactatcttttaacgtaaaagtatcattgttgtttaatgagtaaaaacttaataagctgacggttagatattgacatattaatgtgtcccttagtctaagacaaaaataggttttgtagttttcactagtaggtgtggtgtgtgtatattaatcagagagagagaacaaactgacatatctgaccaacctaaacgattaaagtatatatctatatttattaatcatgagaataatcttatttgatgtacatattttattaaataattttatttttaaattttatttttttaatttatattaacttttcattacttttatattattatttttttattaattatatttaacttctatttttttattatttaaattttttagagaaacacaaatgcctctcttcccttaataattataaagataatgagaaataaaataaaattatagaaagggcattgtccccacagtataatgaatcacaccgtgcttcaatggaaggcaacttaattaaaaccggccaatagtttttattgttaattgtacaaaggcttcattggcatgcactcccaaagtcttaatcgtcttcttcaaactccaatccactttttatgctttaaattgttcttactatttgacgtttacgacccttgatggagatcactttgtattttctctctttttgacattgaaagtgcaaccaaaacttcaacttgtgctactcgtttttgtagctcgcaaattctatctacatcttatccatataaaacgataaagctaagtcgattttggtgtaatacaatttataatggaaaaataaattattagactaatattttaatgctatgaggtggaattgtaatagcaagtgcttcttggaactcactcatctttgttgctttaattttttttatgacaacaggccgtgccagtttttctttcagagatcgcaccttcaagaatacgtggagcattgaatatacttttccagttgaatatcacccttggcattctcttttccaaccttgtcaactatgccaccaacaagtaagtttcttgcgtgcgtgctcccaagttcatcctttttgtttgaataaatcacgagtttgtgtgttaaaactaaaaaatgtatagaacaagataaatcatggccattagaagcttcaccaatctccccagtgtgattactcatgcccaggtgaaagcatcacttactcggagtaattggttaatagtgagagtgatgctagtcaaaactttgaaaattgatggcctgattggagtttttctccaaaagtccaaactccaaagagattctgcttcgtagaaaaagaaaactaacacaattatgaccctttctattactccattcaccacccaaaagtcaagcacatgcttgccaactctgcttagtgttttgggtctccataaattgttcaaaagccagaagcaactcaagcaagtactatctgctaacaacaggacaagtgtttgtttctcaccatatgataaagtcccacctctaagaaacgggccacgattattttccttttcatttttttagtgggaatagaaacaataatttttcattttcatgatttcatcaattgttttctgccatttatatcccgtacctctcattttgctcttgcacctctcaaaagattcaagtggataaaaatacatttttaccaaacacttctttccagaacaactattccgaaataggattattcattcggaataattattccaaaataaagttttttttttattccagaatatctattttagagtaaattttcaaagtgtttattctattctaaaatatgaaaaattatattttagaataattgttcacgaagagtattctactagatacattttcagaataactattccaaaatattctgaaacaactattatgtatcctattttgaaatacttgatagaactccccttcccttctcaaaactcaaacttgtaccatcaagctgtcaggccaccgtgtgtcgcatttgcacctccgtgccatcgtgcatcaccttctcgtgtcaacttgggtcgccatgtggaacaatagggaaggtttgaatgataacatttttggtgtagtcaaagagaatgactttgatgttggcgaagatgaataaggtggactccatgttgaggaagacgaaggggtataagttgttttcttcatgagggtcgttggtttgaacgacgaagggaagagagaaaatatttttggcgtggtgtgggttttgggaaagaattcgtagttgaattgtctcaactacctatgatgatttggcagtcattaggaaggaggtggttggtggagtaccaatggcgggcagagagggtggcaaaggagagttcgagccaatcataggtaggacagggggagaaggtgcaaactttcttttcaccattgttgaagccatcaatttggacgagggagttgtcaagtgtttgggcagcataaggaaaagggagaggtgatgttagggctgaggatattttttactattgagactttttaggaggtgtagtattcatatcgggaaggtacatgatatagataactagcttaattcccataaatcttggtttggtaaccaatccatcttggtaaaggcccgcatgatccaacctagaaatgttaacttgcttaattgttggttagtgtgaaaatttgaattgacgataaactattgtatccatcattgtttcaataaacattcacaaattttaatagattaataatttattttcctaccaatgcttcaatatttttaaatattatatgaagaaaatattaaataaaataaaaatatttaatattttaaaaacttaagaaacattcaatactactttattaatataaacataaagatttgtaggaagatttagaattgtcgttattcaattatttaatgccagaaaattgatatagacttatcactttgtgcatgtttggattgaagttagaaatgttatgtctatgttttaatcttaatgcaaatccaacggataaaagtaaaatgaaaacaaaatcaagagttcgatcttttgagagaattaattgtcttaaaaatactttttcacttcaaaatcaaacatcactcaaactgtaaagaaacccgtaatctagaattgaataataaaaaacgcaaggcaattttttctcgtgaaaaatcagtgccaacttgttatagataataaaattctaaagcatgttccgttagttttgttttgatatacattgggttgttgttcattgttggtgatgatgactattgttgagcaaggacagaatcaaaggtggatggggttggaggctatctctgggtttgggcggtttaccggcactacttctcacgttgggggctttcttggtggtggacaccccaaacagtctcatcgagcgtggtcacttggaagaaggaaaatctgtgctgagaaagattcgtggcattgacaacattgaacctgagttcttggagcttcttgacgcaagtcgtgtggctaaagaagtcaagcaccctttcaggaatatcctcaagcgcaagaacagacctcaactcgtcatttccattgctctgcaggtcagtgctatgttctcaaacattttcctgtgaattaattacacaatttgttctagacttctggtatctacttgcattgatttggtccctaaaaatttaggtatataatttagtctttgaattatgaacttcattcctttttttatcaactttgtgtatctagtagtaatataacatgtgaccaaaataatgtaggtaagtgggagacgacgactttcgtacatgagttgtgagttaggtctaaatctaaattctaatattaagagtctcctttaatatatcttgttgggtttattaggccaattgttattgaacccttatttaatcacctacatatatggccaatatttgatgtgagagtggtgtggtgaagattgtgcattgaataataaacaagaccaatgatagcaatagcatcagtccttgaatttattgtgaagatgacattttagagactaaattctagaccaaattaacatgattaaaacaatgttattgtatgttcactctcttcttaacataattgaaacaatgctattgttagctggatcaatattgatcaattgaattttccagattgatctgattcttattccatttgggtgttttcaaaaaatagatttttcagcagttcactggcatcaatgccataatgttttatgccccggttttgtttaacacattgggtttcaagaacgatgcttctctctactctgctgtgattactggtgctgttaatgtcgtctccactgttgtctcaatctattcagtagacagactcgggaggaaaatgctcttattggaagctggagctcaaatgttcctgtctcaattagtgattgcagtcataataggaatgaaggtaaaggaccattcagaggacctctcaaagggttttgcagtgctagtggttgtgctggtgtgcatttttgtgtctgcatttgcatggtcatggggccctcttagttggctcatcccaagtgagatattcccactggagactcgctcagccgggcaaagtatcgcggtgtgtgttaacttgctatgcacatttgtcattgcacaggcctttctctcaatgctgtgcttcttcaagtttggcatcttcttgttcttctctggatgtgtcttgatcatgtccaccttcgtgcttctcctgcttcccgaaacaaagaatgtccccattgaagagatgactgagagagtgtggaagcagcattggttgtggaacaggtttattgatgaagatgattgtgttaaagaagagaaagtagtcactggcaattga

SEQ ID NO: 38 - DNA, 2188 bp, Glycine max
  nucleic acid for wild type Lr67 gene SEQ ID NO. 7 (Glyma.1 0G249400.1.Wm82.a2.v1)
atggcgggaggaggatttgtatcagcttcaggggaatcccactttgaggcaaaaatcacgtttgccgtcataatatcttgcataatggctgccaccggtggccttatgtttggttatgatattggaatttcaggtacacatgatatgatattgcttagttatgcatttattaatgaatcacattcatgattaaattaaattgcatcaaagaaatgagatattatgaattatatatattgcttgttttgatttcaagctattaaattagtgcatatccaaggctataaacaaaatcatacttaatgttgtgaattgcatgtgaatgcaggtggtgtcacatcaatgcctagctttctagaaaagtttttccctgaggtatacagaaagatacaagatcatggtgtggacagtaactactgcaaatatgacaatcaaactctacaattgttcacatcctctctgtacctcgcagctttggtagccaccatgtttgcctcttccgtcaccaggacgctaggccggaagcaaactatgttgattgctggcatttttttcatcgttggaactgtcttgaatgctgtagcaaatagcttgctactgctaattgttggcagaatcttacttggttgtggtgttggtttcgccaatcaggtccttattatgtattccatttttaatcatactttcaatttctaatgattaatggtttgatacaattatttaaggtataggattttagacatggtcttttgtcctatttatttatgttgttgattaaatgtgtttttcacaataggctgtgccagtgtttatttctgagattgcacccacaagaattcgtggagcacttaacatcatgttccagcttaatatcaccattggaattctcatagcaaacattgtcaactacttcacagcaaagtaagtaatttaatgatccttgtataatactagcaaattgacttggatttgaaggcgcacaaattaagattacatgaaacattttatataactaattaatgaatgtgaattggggagtgtaggattgaaggtggatatgggtggaggatatcagtggctttggcagggattccagcaataatgctgacatttgggtccctcctggtgcatgacactcccaacagtcttattgagaggggtttggaggatgaagggaaggctgtgctaaagaagattcgtggtgtggaaaatgttgagccagagtttcaggagattcttaaagccagtaaagtggctaaagcggttaagaatcccttccaaaacctccttaagcgccacaaccgtcctccattgatcattgccgttatgatgcaggtaacaaattaacactcattgttcttttttttttgttagaaacatcaataagagatattcaaaccatgatctgtttccctcccttctctcttcactcttcccccgacattagactaatcttatatctcacacttattattcatatttggtaacaatatttgaactcataaacttaattaatattttgacaattttttttttaataaattttgttttggtgtaggtcttccaacaattcaccggcatcaatgcgatcatgttctatgctccagtgttgttcagcaccttgggatttaaaagtgatgcttccctttactcagccgtgatcacaggagctgtgaatgtgctatcaaccttggtgtctgtttatttcgtggacaaagctggtcggaggatgctgttgttggaggcctgtgtccaaatgtttgtgtctcagatggtgattggcacagtgcttgggttgaaggtccaagaccactcagatagcttgaacaaagggctcggggtgttggtggtggtcatggtttgcacctttgtggcatcttttgcatggtcttggggaccacttgggtggctcattccgagtgagacattcccacttgaggctaggtcagcggggcaaagtgttaccgtgtttacgaacatgctcttcacatttattattgctcaaggtttcttgtccatgatgtgccacctcaaattcgggatcttctttttcttctccgcctgggtgcttgccatggccatattcacagtgttgctaattccagagactaagaacatacccatagaggagatgacagataaagtctggagaaaccactggttctggaagagttatatggaagattaa